(19) United States
(12) Patent Application Publication
(10) Pub. No.: US 2005/0053642 A1
(43) Pub. Date: Mar. 10, 2005

(54) Biocompatible materials

(76) Inventors: Mathias Ulbricht, Berlin (DE); Volkmar Thom, Arlington, MA (US); Katja Jankova, Burgas (BG); George Altankov, Sofia (BG); Gunnar Jonsson, Vaerloese (DK)

Correspondence Address: Browdy and Neimark;Suite 300, 624 Ninth Street NW, Washington, DC, 20001, US

(21) Appl. No.: 10/362,677

(22) PCT Filed: Aug. 23, 2001
(86) PCT No.: PCT/DK01/00557

(30) Foreign Application Priority Data
Aug. 23, 2000 (DK) PA 2000 01250

(51) Int. Cl. A61L 33/00

(52) U.S. Cl. 424/443

(57) ABSTRACT

The present invention teaches a novel approach of creating biocmpatible surfaces, said surfaces being capable of functionally interact with biological material. SAid biocompatible surfaces comrise at least two comonents, such as a hydrophobic substratum and a macromolecule of hydrophilic nature, which, in a cooperativity, form together the novel biocoompatible surfaces. The novel approach is ased on contacting said hydrophobic substratum with a laterally patterned monomolecular layer of said hydrophilic and flexible macromolecules, exhibiting a pronounced excluded volume. The htus formed two component surface is, in respect to polarity and morphology, a molecularly heterogeneous surface. Structural features of said macromolecular monolayer (as e.g. the layer thickness or its lateral density) are determined by: i) the structural features of the layer forming macromolecules (as e.g. their MW or their molecular architecture) and ii) the method of creating said monomolecular layer (as e.g. by physi- or chemisorbing, or by chemically binding said macromolecules). The structural features of the layer forming macromolecules(s) is in turn determined by synthesis. AMount and conformation and thus also biological activity of biological material (as e.g. polypeptides) which contact the novel biocompatible surface, is determined and maintained by the cooperative action of the underlying hydrophobic substratum and the macromolecular layer. In this way it becomes possible to maintain and control biological interactions between said contacted polypeptides and other biological compounds as e.g. cells, antibodies and the like. Consequently, the present invention aims to reduce and/or eliminate the deactivation and/or denaturation associated with the contacting of polypeptides and/or other biological material to a hydrophobic substratum surface.

### TECHNICAL FIELD OF THE INVENTION

The invention is in the area of biomaterials, i.e. those materials that are used in contact with living tissue and biological fluids for prosthetic, therapeutic, storage or other applications. The working environments of any biomaterial are either biological fluids or living tissue, and the events occurring at the contacting interface playa crucial role in the overall performance of a biomaterial. Many conventional biomaterials lack the ability to properly interact with or support biological matter coming into contact with said biomaterials leading to undesired biological responses. However, these undesired responses may be controlled by altering the chemical and physical properties of the surface of said biomaterials. In this respect, surface modification represents a well known strategy of providing suitable biocompatible materials. The present invention teaches a novel approach of creating biomaterial surfaces, said surfaces being capable of functionally interacting with biological material.

### BACKGROUND OF THE INVENTION

When biological and synthetic materials interact with each other, one must contemplate that an association is formed that is normally not part of a biological environment such as e.g. the human or animal body. A biocompatible material has been defined as a material that, when interacting with biological material, does not induce an acute or chronic inflammatory response and does not prevent a proper differentiation of implant-surrounding tissues.^{[1]}Furthermore, according to another current understanding, are biocompatible materials capable of i) controlling or guiding cell growth and tissue organization, ii) promoting or inhibiting cell-cell or cell-tissue interactions,^{[2]} iii) isolating transplanted cells from the host immune system,^{[3]} and iv) regulating production and/or secretion of cellular products. However, many synthetic materials which are used as biomaterials are not biocompatible according to this definition, and many efforts are undertaken to find ways to improve the biocomnpatibility of these materials.

The quality of the interactions of a synthetic material surface with biological material, i.e. the biocompatibility of said material surface, can be related to the behavior of living cells when in contact with said surface. Accordingly, criteria like the amount of adhered cells, overall cell morphology, cell migration, focal adhesion formation, extra cellular matrix (ECM) formation, and cell proliferation on the material surface are considered important when aiming to monitor and control the biocompatibility of a material surface in vitro.

One important example for the interaction of biological and synthetic materials, is the adhesion of human or animal cells to polymer substrata: Cell adhesion is known to involve various adhesive proteins, such as e.g. fibronectin (FN) and vitronectin (VN), that are adsorbed to the surface of the synthetic material and mediate a contact between said surface and adhering cells.^{[4,5,6,7,8]} These interactions are furthermore mediated by specific transmembrane receptors belonging to the integrin family of cell adhesion molecules. ^{[}9,10] Adsorption of proteins from biological fluids onto a surface of a polymer is dependent on the physico-chemical properties of said polymer surface. ^{[5,15]} For example, it is well known that adhesive proteins adsorb abundantly onto hydrophobic polymer surfaces, but their adsorption, mainly driven by hydrophobic interactions, leads to conformational alterations and eventually to their deactivation and/or denaturation (see FIG. 1).^{[7,12]} These conformational alterations of the adhesive protein explain the reduced or eliminated interaction between said adhesive protein and a cell,^{[}^{13]} leading to reduced polymer—cell interactions.

Furthermore, conformational alterations of proteins that adsorb to the surface of a synthetic material, as. e.g. the surface of an implant or of a medical device, may also give rise to increased thrombogenicity of said material or to foreign body reactions and consecutive rejection of the implantation or medical device.

Synthetic polymers are a class of materials frequently used as biomaterials with selection criteria based on their mechanical properties, stability, and capabilities of producing predefined or desired shapes and/or morphologies. However, these materials are often not biocompatible. For example, synthetic polymers in current use for the preparation of membranes with controlled permeability, e.g. polysulfones, polyesters or polypropylene, are often less than adequate for the immobilization of tissue cells because the functionality of these cells cannot, due to the above described reasons, be maintained over sufficiently long periods of time.

However, the biocompatibility of any substratum may be controlled by altering the chemical and physical properties of said substratum. Surface modification represents a well known strategy of providing suitable biocompatible materials.

Hence, polymer surfaces are e.g. modified through the addition of charged side-groups to the polymer backbone,^{[14]} adsorption or covalent immobilization of biologically active proteins and peptides to the polymer,^{[16]} and the alteration of the texture or morphology of the polymeric substrate.^{[17,18]}

It is known, that surface modification is of particular interest when performed using e.g. a selective reaction initiated under mild conditions, such as e.g. photo-grafting.^{[19]} Using a selective reaction, the shape of the substratum, including macro- or microporous structures, as well as mechanical properties, can be established and/or preserved. Examples of surface functionalizations include macroscopically homogeneous polymeric surfaces that may i) repel cells due to charge, hydrophilicity/flexibility,^{[21]} or surfaces to which ii) cells may adhere via e.g. conditioning of a protein on a hydrophohic surface or via attachment or operable linkage of the protein to a peptide mimicking the binding domain of an adhesive protein.^{[14,21]}

Patterns of functionalization on a m-scale are well suited to create patterns capable of attaching cells as well as patterns of cell-free areas.^{[22]} Furthermore, patterns on sub-μm scale, made up e.g. by a mixture of adhesive peptides and charged groups,[²³] are also suited as supports for cell cultures. Photo-grafting in combination with photo-lithographic techniques^{[24,25]} is an established way of achieving such patterns.

An important general class of surface modification is the attachment of macromolecules to the underlying surface. Often, these macromolecules will exhibit hydrophilic properties and thus be solvated in an biological environment, whereas the underlying surface is e.g. of hydrophobic character. The attachment of macromolecules can be achieved through the i) physical adsorption of amphiphilic macromolecules, ii) use of self-assembled monolayers (SAM),^{[26,27]} iii) ionic binding of charged macromolecules to surface-bound countercharges, iv) grafting of either photo- or thermally-reactive macromolecules,^{[24,28]} and, in the case were the underlying surface is polymeric, also through the, v) entanglement of said macromolecules into the polymer surface. (see also ^{[29,30]})

It is known to coat and shield surfaces of hydrophobic basis polymers with e.g. layers or chains of (attached) hydrophilic macromolecules in order to exclude biological material such as proteins and consequently also cells from coming into contact with said surface. In particular well known are the shielding properties of poly(ethylene glycol) (PEG), an established hydrophilic polymer. The protein repellent character of substrata coated with PEGs is accredited to a combination of several molecular mechanisms,^{[4]} where consensus seems to be reached that the steric stabilization forces induced by the excluded volume of the attached macromolecules represents the dominating mechanisin.^{[31,32]} It has been observed, that the protein repelling character of PEG-coated substrata is dependent on their lateral density on a substratum surface,^{[33,34]} where a correlation between amount of adsorbed protein and lateral density was observed: the higher the lateral density of attached PEGs, the lower the adsorption of proteins. According to the above, different techniques are known which describe how to prepare coated substrata, where the coats effectively shield the underlying substratum, and where said coats can also be laterally patterned.

Recently, Sofia et al.^{[33]} characterized protein (FN, cytochrome-c, and albumin) adsorption on PEG-grafted (molecular weights (MW): 3.4, 10, and 20 kilo Dalton (kDa)) silicon surfaces over a range of grafling densities. Additionally, the grafted amount of PEG moieties could be measured with Electron Spectroscopy for Chemical Analysis (ESCA) Protein adsorption decreased for all proteins with rising PEG grafting density and was reduced by more than 95% for the highest PEG grafting density when compared to the unmodified silicon substrate. From measurements of the thickness of the adsorbed protein layer it was deduced, that FN, having a rod like shape, adsorbs for all PEG grafting densities “lying down” with its long axis parallel and in close contact to the surface. Thus, proteins are able to penetrate the PEG-layer to effectively adsorb to the underlying surface in between grafted chains. Sofia et al. calculated that protein adsorption started to decrease for grafting densities were grafted PEG chains began to overlap, and that protein adsorption became negligible when PEG chains were, due to the grafting density, confined to approximately half of their relaxed volume. This correlation between protein adsorption and PEG chain overlap was found for all investigated PEG MWs, and the calculations were based on the assumption, that grafted PEG chains exert the same radius of gyration or spatial dimension as in their solvated state.

Contact angles (CA) are generally used to characterize the wettability of surfaces. Wettability of a surface is related to its hydrophilicity as constituted by the moieties forming that surface. It is a very sensitive technique with a probing depth of approx. 5-10 Ångstrøms. CA are determined by measuring the angle defined by the phase limits of the liquid phase at a three phase boundary (solid/liquid/vapor). The three phase boundary is generated by e.g. a vapor bubble in a liquid, where the bubble is captivated by the test surface (captive-bubble method, see also FIG. 2) or a drop of liquid in vapor placed on top of the test surface (sessile-drop method). When a polar (hydrophilic) test liquid (as e.g. water) is used, hydrophilic test surfaces will generate small angle values such as 00-90°, while hydrophobic test surfaces will generate large angle values of 90°-180°. Both advancing and receding measurements of CA's are used to characterize the biomaterials according to the invention.^{[35]}

While it is not intended that the present invention be limited by the nature of the particular mechanism or the understanding of the particular physical forces involved, a drop of liquid resting on a substratum may be considered to be balancing three forces: a) the interfacial tension between the solid and liquid, b) the interfacial tension between the solid and vapor, and c) the interfacial tension between the liquid and the vapor. The angle within the liquid phase is known as the “contact angle”. See B. C. Nayar and A. W. Adamson, “Contact Angle in Industry”. Science Reporter, pp. 76-79 (February 1981). It is the angle included between the tangent lane to the surface of the liquid and the tangent plane to the surface of the solid at an point along their line of contact

Advancing and receding contact angles are frequently found not to be the same. This hysteresis may be due to rough surfaces or to chemical heterogeneities of the substratum.

One method of measuring the contact angle is by taking a photograph of a bubble captivated by the substratum and then measuring the angle from the print. The angle can also be measured from an enlarged image of the bubble. A low power microscope produces a sharply defined image of the liquid bubble which is observed through the eyepiece as a silhouette.

There are commercially available goniometers with environmental chambers in which contact angles can be determined in controlled conditions of temperature and pressure. A camera can also be attached to such goniometers. Commercial instruments for measuring contact angles are available from such companies as RAME-HART, Inc. (Mountain Lakes, N.J.), KRUSS (Charlotte, N.C.), CAHN INSTRUMENTS (Cerritos, Calif.), and KERNCO INSTRUMENTS (El Paso, Tex.).

Contact angles ranges from zero (0) to one hundred and eighty (180) degrees (although the latter is not encountered in practice). In this invention, water is used as a probing liquid (representing the liquid phase), and air saturated by water vapor as the gas phase (forming the bubble). When the contact angle is between approximately zero and approximately ninety (90) degrees, the substratum is considered hydrophilic. Ninety (90) degrees is considered “hydroneutral.” When the contact angle is greater than ninety (90) degrees, the substratum is considered hydrophobic.

Ellipsometry is an optical in situ technique for measuring i) the refractive index of a bare surface, or ii) the thickness and refractive index of a film/coat on a substratum, both based on measuring the change in the state of polarization of light upon reflection from said substratum surface. The determined thickness and refractive index of an adsorbed layer of macromolecules can thus be converted to a value of adsorbed mass.[36] In this way it is possible to monitor on-line the adsorption of e.g. macromolecules out of solution onto a substratum surface interfacing that solution. There are detailed descriptions of the physical principles of the method[37] and the instrumental setup.[38]

The below-identified documents form part of the prior art.
Thom et al. (2000), Langmuir 16 (2000) 2756-2765, discloses the interaction between a modified polysulfone substratum, fibronectin and human fibroblasts.
Defife et al. (1999), J. Biomed. Mater. Res. 44(3): 298-307, discloses suppression of fibrogen adsorption and IgG toga silicone rubber using a photochemical immobilization technique.
Defife et al. (1999) J. Biomaterials Sci. Polymer Ed. 10(10) 1011-1162, discloses abolition of fibroblast growth on modified silicone rubber.
Zziampazis et al. (2000) Biomaterials 21 511-520, discloses the role of polyethylene glycol (PEG) in modifying the surface properties of a modified polycarbonate.
Sofia et al. (1998), Macromolecules 31, 5059-5070, compares different PEG type molecules, and their interaction with proteins when chemically grafted to a polymer substratum.
U.S. Pat. No. 5,776,748 is related to a device comprising a plurality of cytophilic islands and cytophobic regions established by self-assembled monolayers exhibiting cytophilic or cytophobic endgroups. Cell-adhesion is promoted or inhibited on the cytophilic or cytophobic regions respectively by known mechanism, as e.g. introduction of polar groups, charges, and the like.
U.S. Pat. No. 5,002,582 is related to a method of producing biomaterials having an “effective” solid surface characterized by the properties of the hydrophilic polymer and not of the solid hydrophobic surface (column 8). The claimed biomaterials do not have a contact angle that is substantially similar to that of the solid surface.
U.S. Pat. No. 4,973,493 is related to a method of producing a solid surface that is effectively shielded by a biocompatible agent. The claimed biomaterials are unlikely to have a contact angle that is substantially similar to that of the solid surface.
U.S. Pat. No. 4,722,906 is related to a method for selectively binding specific molecular target moieties covalently to a chemical moiety or substratum.
U.S. Pat. No. 5,128,170 is related to a method for manufacturing a medical device having a highly biocompatible surface. The claimed biocompatible surface does not have a contact angle that is substantially similar to that of the medical device.
U.S. Pat. No. 5,728,437 is related to an article comprising a hydrophobic surface coated with a blood compatible surface layer. The coated surface does not have a contact angle that is substantially similar to that of the hydrophobic surface. The document does not disclose the binding of biological material in an active form to the disclosed polymer material.
U.S. Pat. No. 5,380,904 is related to a method for rendering a surface biocompatible. The biocompatible surface does not have a contact angle that is substantially similar to that of the untreated surface.
U.S. Pat. No. 5,512,329 is related to methods of attaching a polymer to a surface of a substrate by application of an external stimulus. The method of claim 14 is directed to a method of modifying surface properties of a substrate. A biomaterial comprising a polymer substratum and a macromolecule and a first determinant capable of bringing a second determinant into contact with said first determinant is not disclosed. Neither does the document disclose the binding of biological material in an active form to the disclosed polymer material.
U.S. Pat. No. 5,217,492 is related to a specialized means for attaching a biomolecule to a hydrophobic surface. The disclosed means for attachment is not pertinent to the present invention.
U.S. Pat. No. 5,263,992 is related to a biocompatible device comprising a solid surface and a biocompatible agent positioned sufficiently proximate to one another so as to effectively shield the solid surface.
U.S. Pat. No. 5,741,881 is related to a bio-active coating that exploits a hydrophilic spacer with functional end groups and capable of linking a specialized polymer with a bio-active agent. The present invention does not exploit a bifunctional linker in the form of a hydrophilic spacer as a means for attaching a first determinant to a polymer substratum.
WO 97/46590 is related to a material comprising a support and two layers, of which the second, outer layer is a hydrophilic polymer, said material further comprising immobilized biological material. The surface generated by coating a support with a polymeric surfactant and hydrophilic polymer does not have a contact angle that is substantially similar to that of the support
WO 97/18904 is related to a method for providing a hydrophobic surface with a hydrophilic coating. The surface generated by hydrophilic coating does not have a contact angle that is substantially similar to that of the hydrophobic surface.
EP 633 031 A1 is related to a composition that is effectively capable of shielding a polymer from biological material. The shielded polymer does not have a contact angle that is substantially similar to that of the unshielded polymer.
Park and Griffith (1998), J. Biomat. Sci. Polym. Ed. 9, p. 89-110, discloses a specialized PEG-PPO-PEG copolymer scaffold capable of effectively inhibiting cell adhesion. The copolymer is useful in regulating the three dimensional organization of diverse cell types. Adhesion is achieved by covalent linkage to the polymer of a cell specific carbohydrate ligand capable of binding a particular receptor moiety. The present invention is not concerned with a polymer substratum being contacted with a first determinant. The cell adhesive properties of the biomaterial according to the present invention are at least partly determined by the cooperativity of a polymer substratum and a macromolecule and optionally also by a first determinant. The polymer “back-bone” of the present invention is not cytophobic per se, as is the case in the cited reference.
Noh et al. (1998), J. Biomat. Sci. Polym. Ed; 9, p. 407426, discloses a modification of PTFE films that substantially alters the contact angle.
Malmsten et al. (1998), J. Coll. and Interface Science 202 , p. 0.507-517, examines the effect of chain density on inhibition of protein adsorption. The document does not mention the properties of the bound proteins, and the document does not disclose the binding of biological material in an active form to the disclosed polymer material.
Zhang et al. (1998), Biomaterials 19 , p. 953-960, discloses silicon surfaces that are modified with a PEG film in order to reduce protein adsorption. The silicon surface does not have a contact angle that is substantially similar to that of the PEG-coated material.
Herbert et al. (1997), Chemistry and Biology 4, p. 731-737, discloses a method of differentiating the cross-linking of bioactive molecules to a surface. Biomaterials according to the present invention are not disclosed and the disclosed method is not pertinent to the present invention as photo-reactivation is acknowledged to form part of the prior art.
Wesslén et al. (1994), Biomaterials 15, p. 278-284, discloses a surface modification of a hydrophobic polymer by use of hydrophilic polymers including PEG. The modification significantly changes the contact angles (Table 1) and leads to a reduced polypeptide adhesion.
Bergström et al. (1992), J. Biomedical Materials Research 6, p. 779-790, discloses a polystyrene comprising densely packed and covalently bound PEG capable of effectively reducing adsorption of fibrinogen. The polystyrene does not have a contact angle that is substantially similar to that of the densely packed PEG surface.
Desai and Hubbell (1991), Biomaterials 12, p. 144-153, discloses an incorporation of PEG and similar water-soluble polymers onto surfaces of biomedical polymers such as e.g. PET and the like. The incorporation significantly alters the contact angle as illustrated in Table 1.
Gombotz et al. (19901), J. Biomedical Materials Research, 25 , p. 1547-1562, discloses a modification of PET surfaces with PEG. The incorporation significantly alters the contact angle as illustrated in and the first paragraph of the discussion.

### SUMMARY OF THE INVENTION

The present invention teaches a novel approach of creating biocompatible surfaces, said surfaces being capable of functionally interact with biological material. Said biocompatible surfaces comprise at least two components, such as a hydrophobic substratum and a macromolecule of hydrophilic nature, which, in a cooperativity, form together the novel biocompatible surfaces.

The novel approach is based on contacting said hydrophobic substratum with a laterally patterned monomolecular layer of said hydrophilic and flexible macromolecules, exhibiting a pronounced excluded volume. The thus formed two component surface is, in respect to polarity and morphology, a molecularly heterogeneous surface. Structural features of said macromolecular monolayer (as e.g. the layer thickness or its lateral density) are determined by, i) the structural features of the layer forming macromolecules (as e.g. their MW or their molecular architecture) and, ii) the method of creating said monomolecular layer (as e.g. by physi- or chemisorbing, or by chemically binding said macromolecules). The structural features of the layer forming macromolecule(s) is in turn determined by synthesis.

Amount and conformation and thus also biological activity, of biological material (as e.g. polypeptides) which contact the novel biocompatible surface, is determined and maintained by the cooperative action of the underlying hydrophobic substratum and the macromolecular layer. In this way it becomes possible to maintain and control biological interactions between said contacted polypeptides and other biological compounds as e.g. cells, antibodies and the like. Consequently, the present invention aims to reduce and/or eliminate the deactivation and/or denaturation associated with the contacting of polypeptides and/or other biological material to a hydrophobic substratum surface.

In a preferred hypothesis, solvated polypeptides penetrate the laterally patterned monolayer of macromolecules to effectively adsorb in-between said macromolecules to the underlying hydrophobic surface. Said polypeptides must, in order to penetrate the monolayer of macromolecules, deform said self-assembled macromolecules to some degree, inducing a lateral pressure acting between said macromolecules and penetrated polypeptides, but also between said macromolecules themselves (see also FIG. 3). This lateral pressure has its origin in the unfavorable loss in conformational entropy of said bound macromolecules related to the spatial deformation of said macromolecules. The lateral pressure will therefore increase as the amount of penetrated polypeptides increases.

Consequently, the amount of adsorbed polypeptides will, according to the hypothesis, continue to increase until an energetically favorable balance is attained between, i) the unfavorable induced lateral pressure, and ii) the favorable adsorption of said polypeptides to the underlying hydrophobic surface. Polypeptides will therefore continue to penetrate the macromolecular layer to effectively adsorb to the underlying hydrophobic surface until the hereby induced lateral pressure in that layer will effectively repel any other polypeptides from that layer.

According to this hypothesis, polypeptides adsorbed in-between said self-assembled macromolecular layer will be exposed to a lateral pressure originating from surrounding and deformed macromolecules. The lateral pressure acting upon adsorbed polypeptides, will effectively protect said polypeptides from unfolding/denaturation, and stabilize said polypeptides in an active conformation, yielding adsorbed but bio logically active polypeptides.

The invention thus solves the problem of how to provide—by simple and inexpensive methods—general surface design principles and modification methods in order to enable e.g. the control of attachment, spreading, growth and tissue formation of cells on surfaces, as these depend on biologically active polypeptides present at a surface. These novel biocompatible surfaces may thus be used as cell-culture dishes, bioreactors, implants, biohybrid organs such as pacemakers, and the like, without the need of extensive development of new polymers and biocompatibility screening.

It is therefore contemplated, that the present invention provides means to create bio compatible surfaces suitable for use in emerging technologies such as e.g. the construction and application of novel surface architectures of biomaterials with innovative functionalities. Accordingly, the invention is useful in the manufacture of surface architectures for use in biohybrid organs, such as e.g. a bioartificial pancreas, liver or kidney. The invention will enable the use of improved membranes for ensuring spatial separation of e.g. xenogenicu and/or allogenic cells from the host immune system.

Modifying membranes with said macromolecular layers comprising hydrophilic macromolecules such as e.g. PEG may according to the present invention reduce the amount of adsorption of proteins on the plane of the membrane^{[24]} and at the same time improve the conformational/functional state/form of adsorbed proteins such as FN and other attachment proteins.

The present invention also contemplates providing arrays for culturing “sensual” cells such as e.g. nerve, olfactorial, retina, and similar cells. Culturing of sensual cells requires a spatially resolved reception of signals that must be organized in a highly complex and specific manner. The signals generated by those cells must be transmitted to a non-biological support in a time resolved and location dependent manner. Photolitographical techniques involving e.g. the immobilization of PEG spacers and bio-specific ligands may be used to contribute to the structuring and/or functionalization of solid supports in a highly specific way. It is envisaged that such structures may eventually be used e.g. as sensors or biohybrid organs.

Cells capable of being immobilized onto the biomaterials according to the invention ate preferably, but not limited to, cells the function of which comprise i) controlled delivery of biologically active substances, such as e.g. hormones, ii) production of predetermined proteins and polypeptides derivable therefrom, such as e.g. antibodies, growth factors, matrix factors, and the lice, or iii) the conversion of metabolites, preferably toxic or cytostatic metabolites. Examples for such types of cells are e.g. Langerhans islets cells, hybridoma cells, chondrocytes, and hepatocytes.

It is contemplated that the invention is useful in the organization of cells in organs and tissues. Such an organization involves a controlled co-operation of different types of cells that are connected, on a micrometer scale, through a local and highly organized network of different cell types. It is contemplated that the present invention will allow photolitographical techniques to be applied in the immobilization of macromolecules with distinct functionalities and biogenic ligands. The biomaterials thus generated are capable of immobilizing different types of cells in a controlled and/or spatially structured manner so as to make them available for a controlled co-operation.

It is also contemplated to obtain an organization of cells in organs and tissue-like structures by stochastically distributed macromolecules (e.g. with and without specific functionalities, such as, e.g. amine groups, either itself or for subsequent immobilization of biological or biomimetic receptors) on a solid support, and subsequently use a second ligand (e.g. another macromolecules with a different functionality such as e.g. a functionality exerted by e.g. a different chain length) in the formation of clusters of different sizes (e.g. clusters with a different length with regard to an axis, e.g. the z-axis) and/or functionality. In this way, the invention makes it possible to obtain a patterning of a given substrate in three, dimensions. This may eventually offer the possibility of providing structured surfaces for the immobilization of e.g. a single type of cells, or e.g. co-culture different cells by binding ligands that are selective for specific cell surface receptors, such as integrins, growth factor receptors and the like.

The novel and innovative applications described herein above cannot be realized with the state of the art means currently available, because there exists a profound lack of useful design principles and suitable methods for surface modification. Also, the state of the art methods are not readily applicable to fine-tune the surface structure and/or biocompatibility of known polymeric biomaterials. The invention described herein represents a significant improvement of the state of the art techniques and potentially enables the creation of novel biocompatible materials and cell-based technologies.

According to one preferred aspect of the present invention, the biocompatible material surface has a contact angle that is substantially identical to the contact angle of the underlying hydrophobic substratum of said surface. Substantially identical contact angles within the meaning of the present invention will be understood to comprise any change of contact angle within the numerical value of less, than 5 degrees, such as less than 4.5 degrees, for example less than 4.0 degrees, such as less than 3.7 degrees, for example less than 3.3 degrees, such as less than 3.0 degrees, for example less than 2.8 degrees, such as less than 2.5 degrees.

The biocompatible surface according to the invention differs from prior art hydrophobic substrata that are coated with a hydrophilic layer, as such prior art surfaces have a contact angle that is significantly different from that of the basis substratum. Consequently, the invention relates to conversion of a hydrophobic substratum having a predetermined contact angle into a biocompatible material surface having essentially the same contact angle but having another functionality with respect to biologically active moities, such as polypeptides, proteins, cells, etc. being in contact with said substratum. The biocompatible surface may further comprise a first determinant, e.g. an adhesion polypeptide, capable of bringing a second determinant, e.g. a biological cell, into reactive contact with said first determinant.

In yet another aspect of the invention, the biocompatible surface is capable of interacting with at least one first determinant (e.g. a polypeptide) and maintainsaid first determinant in an active form, preferably an active conformation. The presence of said first determinant in its functional form and/or active conformation results in an improved first determinant-mediated contact between said biocompatible surface comprising said first determinant and e.g. a second determinant such as a cell capable of contacting said first determinant and preferably forming a stable association therewith

The first and second determinant may in one embodiment independently of one another comprise a cell or consist of a cell. The cell is preferably selected from the group consisting of adipocytes, astrocytes, cardiac muscle cells, chondrocytes, endothelial cells, epithelial cells, fibroblasts, gangliocytes, glandular cells, glial cells, hematopoietic cells, hepatocytes, keratinocytes, myoblasts, neural cells, osteoblasts, pancreatic beta cells, renal cells, smooth muscle cells, striated muscle cells, and precursors of any of the above. Additionally preferred cells are stromal tissue cells found in loose connective tissue or bone marrow, and preferably endothelial cells, pericytes, macrophages, monocytes, leukocytes, plasma cells, mast cells, including any precursor thereof.

When the second determinant is a cell, preferably any one or more of the ones listed herein immediately above, the first determinant preferably comprises a polypeptide or another biological entity capable of optimising or stabilising the association formed between the material according to the invention and the cells in question.

In a first aspect of the invention there is provided a biocompatible material comprising a substratum contacted by at least one macromolecule,
said material having a first advancing contact angle a,
said substratum having a second advancing contact angle b₀ when not contacted by a macromolecule, and another second advancing contact angle bₛₐₜ, when said substratum is saturated by said macromolecules,
wherein said advancing contact angles are measured using water and air saturated by water vapour,
wherein bₛₐₜ essentially does not change when the substratum is contacted by further macromolecules by means of a chemical bond,
wherein the relation between said advancing contact angles is as defined by the ratio R,
   R=(b₀−a)/(b₀−bₛₐₜ)
   and wherein the numerical value of R is in the interval from 0 to less than 0.4, such as less than 0.38, for example less than 0,36, such as less than 0.34, for example less than 0.32, such as less than 0.30, for example less than 0,28, such as less than 0.26, for example less than 0.24, such as less than 0.22, for example less than 0.20, such as less than 0.18, for example less than 0.16, such as less than 0.15, for example less than 0.14, such as less than 0.13, for example less than 0,12, such as less than 0.11, for example less than 0.10, such as less than 0.09, for example less than 0.08, such as less than 0.07, for example less than 0.06, such as less than 0.05, for example less than 0.048; such as less than 0.046; for example less than 0.044, such as less than 0.042, for example less than 0.040, such as less than 0.038, for example less than 0.036; such as less than 0.034, for example less than 0.030; such as less than 0.028, for example less than 0.026; such as less than 0.024; such as less than 0.022; for example less than 0.020, such as less than 0.018; for example less than 0.016, such as less than 0.014; for example less tub 0.012, such as less than 0.010; for example less than 0.008; such as less than 0.006; for example less than 0.004, such as less than 0.002, for example less than 0.001.

A ratio R of 0 (zero) does not occur theoretically as the contacting angles a and b₀ are different (values of) contacting angles and do not attain the same value. However, measurement errors of only very slightly modified substrata can contribute to ratios R of essentially zero.

In another aspect the present invention pertains to a material comprising a substratum, said substratum being contactable with a macromolecule, said material further comprising at least one macromolecule,
said material having a first contact angle a,
said substratum having a second contact angle b₀ when not contacted by a macromolecule,
said contact angle a being substantially identical to said contact angle b₀.

In one embodiment of this aspect there is provided a material comprising a substratum,
said substratum being contactable with a macromolecule; said material further comprising at least one macromolecule,
said material having a first contact angle a,
said substratum having a second contact angle b₀ when not contacted by a macromolecule, and another second contact angle bₛₐₜ, when said substratum is saturated by said macromolecules as defined herein,
wherein the relation between said contact angles is as defined by the ratio R,
   R=(b₀−a)/(b₀−bₛₐₜ)
and wherein the numerical value of R is in the interval from and including 0 to less than 0.4.

In another aspect the invention pertains to a material having a first contact angle and comprising a substratum having a second contact angle, said substratum being contacted by a plurality of soluble substances capable of forming a self-assembled monolayer comprising a macromolecule and having a third contact angle, wherein the relation between said contact angles as defined by the ratio between
i) the difference between the third contact angle of said monolayer, when no macromolecule is present, and said first contact angle, and
ii) the difference between the third contact angle of said monolayer, when no macromolecule is present, and the contact angle of said self-assembled monolayer, when said monolayer is saturated by said macromolecules as defined herein,
   is more than −0.6 and less than 0.6.

All contact angles used to characterize the material are advancing contact angles. Pure water is used as probing liquid, and air saturated with water vapor, is used as probing gas. The material/substratum, the pure and furthermore double distilled water and the air saturated with water vapor, will form the three-phase boundary used to measure the contact angle.

The described properties of a biocompatible surface according to the invention comprising said hydrophobic substratum and said hydrophilic macromolecule allows a first determinant to adhere to and remain associated with said surface in a functional conformation or a biologically active form or conformation. The properties of said surface comprising said substratum and said macromolecule and said first determinant are also useful, as a second determinant can adhere to and remain associated in a functional or active form or conformation, preferably a biologically active form or conformation, with said first determinant and consequently with the surface.

### FIGURE LEGENDS

FIG. 1 An adsorbed/immobilized biologically active moiety (e.g. a polypeptide) becomes conformationally altered (with time) and thus inactive due to attractive (e.g. hydrophobic) interactions between the underlying substratum and the adsorbed/immobilized polypeptide

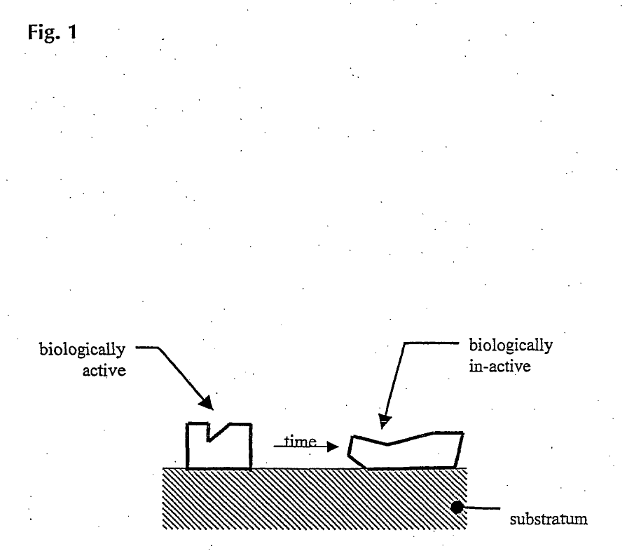

FIG. 2 A schematic showing how to measure contact angles at a three phase boundary, i.e. substratum (solid), water (liquid), and water vapour (gas). A bubble of water vapour is captivated by the above horizontal substratum which is immersed into water.

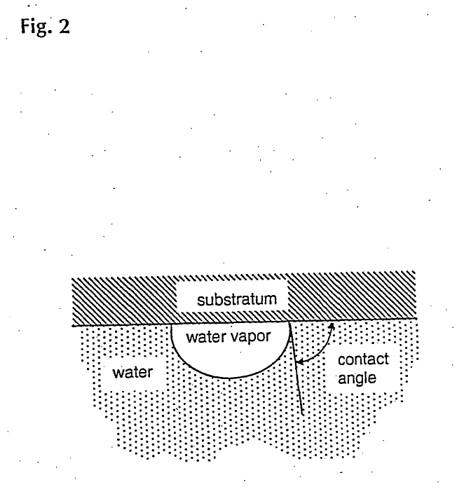

FIG. 3 Immobilized hydrophilic macromolecules neighboring interstitially adsorbed/immobilized moities (e.g. a polypeptide) excert a lateral pressure upon said moities stabilizing them in their active conformation

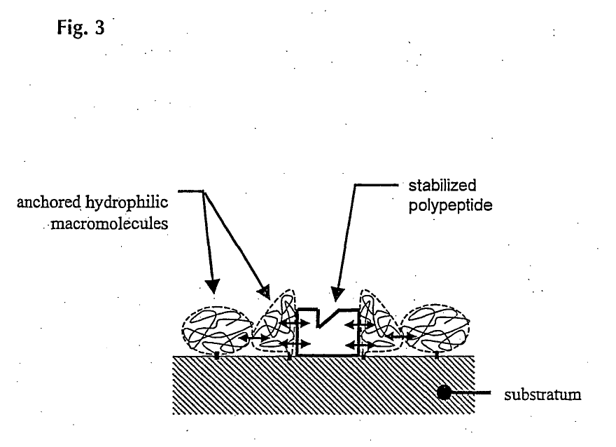

FIG. 4A two-step polymer surface functionalization procedure

FIG. 5 Hydrophilic macromolecules, neighboring interstitially adsorbed/immobilized moities, are immobilized to the underlying substratum by means of chemical bonds.

FIG. 6 Hydrophilic macromolecules, neighboring interstitially adsorbed/immobilized moities, are immobilized to the underlying substratum by means of ionic bonds (countercharges).

FIG. 7 Hydrophilic macromolecules, neighboring interstitially absorbed/immobilized moities, are immobilized to the underlying substratum by means of adsorption.

FIG. 8 Hydrophilic macromolecules, neighboring interstitially adsorbed/immobilized moities, are immobilized to the underlying polymer substratum by means of mutual entanglement.

FIG. 9 Hydrophilic macromolecules, neighboring interstitially adsorbed/immobilized moities, are immobilized to an underlying SAM.

FIG. 10 Adsorption kinetics of ABMPEG 5 kDa and MPEG 5 kDa out of aqueous solutions (10 g/l) onto PSf spin-coated on polished silica wafers, as determined by ellipsometry.

FIG. 11 Advancing and receding CA on PSf; spin-coated on glass cover slips and being modified at different ABMPEG 10 kDa bulk concentrations.

FIG. 12 Receding CA and their hysteresis on PSf, spin-coated on glass cover slips and being modified at different concentrations of ABMPEG 10 kDa, ABMPEG 5 kDa, and ABMPEG 2 kDa.

FIG. 13 Table of CA-hysteresis and receding CA relating to FIG. 12 (nd.:no data available).

FIG. 14 Advancing and receding CA on PSf, spin-coated on glass cover slips and being modified with solution mixtures of ABMPEG 2 kDa and ABMPEG 0.10 kDa yielding a total ABMPEG concentration of 10 g/l.

FIG. 15 Receding CA on PSf, spin-coated on glass cover slips and being modified at different concentrations of ABMPEG 10 kDa. CA are shown after modification and after consecutive rinse with isopropanol/water=1/1.

FIG. 16 Adsorbed amount of BSA on an unmodified and ABMPEG 5 kDa modified PSf UF membrane after 2 h static exposure of the membrane to a 0.1 g/l. BSA solution (0.15 molar phosphate buffer, pH=7, room temperature) and consecutive gentle rinsing in buffer.

FIG. 17 Adsorption kinetics of FN to unmodified and ABMPEG 10 kDa modified PSf, spin-coated on polished silicon wafers, as monitored by in-situ ellipsometry

FIG. 18 Overall cell morphology of HF adhering on unmodified PSf or on ABMPEG 10 kDa modified PSf spin-coated on glass cover slips. Effect of ABMPEG 10 kDa density. HF were “plated for 2 h on unmodified PSf (A), or PSf grafted at different ABMPEG, 10 kDa concentrations as follows: (B) 0.001 g/l, (C) 0.01 g/l, (D) 0.1 g/l (E) 1 g/l, 10 g/l. At the end of incubation, simples were investigated and photographed under phase contrast at low magnification (20×).

FIG. 19 Number of adherent HF per microscopic field on unmodified PSf and ABMPEG 10 kDa modified PSf spin-coated-on glass cover slips. Error bars represent standard deviations of the obtained data.

FIG. 20 Focal adhesion formation of HF adhering on unmodified PSf and ABMPEG 10 kDa modified PSf, spin-coated on glass cover slips. Effect of ABMPEG 10 kDa density. HF were plated for 2 h on unmodified PSf (A), or PSf grafted at different ABMPEG 10 kDa concentrations as follows: (B) 0.001 g/l, (C) 0.01 g/l, (D) 0.1 g/l, (E) 1 g/l, (F) 10 g/l. At the end of incubation, the cells were fixed, permeabilized and stained for vinculin by immunofluorescence. Samples were visualized and photographed at high magnification (100×).

FIG. 21 Focal adhesion formation of HF adhering on unmodified PSf and ABMPEG 10 kDa modified PSf, spin-coated on glass cover slips. Effect of serum pre-coating. HF were plated for 2 h on serum-coated unmodified PSf (A), or on serum-coated PSf grafted at different ABMPEG 10 kDa concentrations as follows: (B) 0.001 g/l, (C) 0.01 g/l, (D) 0.1 g/l, (E) 1 g/l, (F) 10 g/l. At the end of incubation, the samples were fixed, permeabilized and stained for vinculin. Samples were visualized and photographed at high magnification (100×).

FIG. 22 FN matrix formation by HF cultured on unmodified PSf and ABMPEG 10 kDa modified PSf; spin-coated on glass cover slips. Effect of PEG density. BF were cultured for 5 days in DMEM containing 10% FBS on: (A) unmodified PSf; or on modified PSf grafted at different ABMPEG 10 kDa concentrations as follows, (B) 0.001 g/l, (C) 0.01 g/l, (D) 0.1 g/l, (E) 1 g/l and (F) 10 g/l. At the end of incubation, the HF were fixed and stained for FN by immunofluorescence. Samples were viewed and photographed at low magnification (25×).

FIG. 23 FN matrix formation by HF cultured on unmodified PSf and on ABMPEG 10 kDa modified PSf surfaces. Effect of ABMPEG 10 kDa density; HF were cultured for 5 days in DMEM containing 10% FBS on: (A) unmodified PSf, or on PSf grafted at different ABMPEG 10 kDa concentrations as follows: (B) 0.001 g/l, (C) 0.0.1 g/l, and (D) 10 g/l. At the end of incubation, the HF were fixed and stained for FN by immunofluorescence. Samples were viewed and photographed at high magnification (100×).

FIG. 24 HF proliferation on unmodified PSf and ABMPEG 10 kDa modified PSf, spin-coated on glass slides. Phase contrast photographs were taken at 1, 3, and 7 days.

FIG. 25 HUVEC proliferation on unmodified PSf and ABMPEG 10 kDa modified PSf, spin-coated on glass slides. Phase contrast photographs were taken at 3, 5, and 7 days.

FIG. 26 C3A proliferation on unmodified PSf and ABMPEG 10 kDa modified PSf, spin-coats on glass slides. Phase contrast photographs were taken at 3, 5, and 7 days.

FIG. 27 XTT assay for HF after 1, 3, and 7 days, cultivated on unmodified PSf and on ABMPEG 10 kDa modified PSf; spin-coated on glass slides and comparted into 8 wells by silicon masks. Error bars represent the standard deviation of the data.

FIG. 28 LDH assay for HF after 1, 3, and 7 days, cultivated on unmodified PSf and on different ABMPEG 10 kDa modified PSf spin-coated on glass slides and comparted into 8 wells by silicon masks. Error bars represent the standard deviation of the data

FIG. 29 Focal adhesion formation of HUVEC adhering on unmodified and ABMPEG 10 kDa modified PSf, spin-coated on glass cover slips. HUVEC were plated for 2 h on FN-coated unmodified PSf (A), or on FN-coated PSf grafted at different ABMPEG 10 kDa concentrations as follows: (B) 0.001 g/, (C) 0.01 g/l, ( ) 0.1 g/l, (E) 1 g/l, (F) 10 g/l. At the end of incubation, the samples were fixed, permeabilized and stained for vinculin. Samples were visualized and photographed at high magnification (100×).

FIG. 30 Ellipsometric data of the consecutive adsorption of i) BGG as antigen, ii) HSA as blocking agent, and iii) a-BGG as respective antibody to BGG to unmodified PSf and PSf modified with ABMPEG 10 kDa at a concentration of 10 g/l. PSf was previously spin-coated on polished silicon slides. Arrows indicate flushing with buffer or addition of concentrates as described in the text.

FIG. 31 Figure a-c shows ellipsometric data of the consecutive adsorption of a) BGG as antigen, b) HSA as blocking agent, and c) a-BGG as respective antibody to BGG, to unmodified PSf and PSf modified with ABMPEG 10 kDa at different concentrations. All values are arithmetic means of two independent experimental runs. Error bars represent the respective standard deviations. PSf was previously spin-coated on polished silicon slides. FIG. 31d shows the ratio between the adsorbed amount of a-BGG in the third step (c) and the bound amount of BGG in the first step (a).

### DETAILED DESCRIPTION OF THE INVENTION

Definitions

Active conformation: protein in a conformation, where it has its normal biological activity in a native host organism
Active form: protein or biological material in a form, where it has the same function as when said protein or biological material is present in native host or native environment.
Adsorption: the taking up of molecules from a gas or liquid on the surface of another substance such as a subs
Advancing contact angle: contact angle when the liquid front is caused to advance over said solid material/substratum. Advancing contact angle may be determined for a substratum per se and/or for a substratum, which has been subject to a pretreatment.
Amphiphil: substance containing both polar, water-soluble and nonpolar, water-insoluble groups.
Arrays for culture of “sensual” cells: Solid or semi-solid supports with ordered structures for the attachment of sensual cells, such as retina cells.
Biocompatible material: Material that, when interacting with biological material, does not induce an acute or chronic inflammatory response and does not prevent a proper differentiation of implant-surrounding tissues.
Biologically active form: see active form.
Biologically active conformation: see active conformation.
Biological material: Any material derived from a living entity including plants, animals or a living part thereof, such as an organ or cell. The preferred biological system is a mammalian system, preferably a human system.
Biomaterial: A material interfacing with biological systems to e.g. evaluate, treat, augment or replace any tissue, organ or function of the body.
Biogenic ligand: Any ligand of biological origin, such as carbohydrates, proteins or parts thereof such as e.g. oligopeptides, including any combination and/or derivatives thereof.
Biohybrid organ: A device comprising a combination of a biomaterial and a biological material in an active form, such as e.g. specific organ cells.
Cell differentiation: Process by which a precursor cell becomes a distinct specialized cell type.
Conformational alterations: Change in the overall three dimensional form of a material, usually a biological material.
Conformational entropy: The entropy of a macromolecule as determined by the amount of possible conformations that the macromolecule may attain.
Conjugate: Plurality of functional molecules chemically joined together.
Contact angle (CA): Angle (θ) represented by the limits of the liquid phase at a three phase boundary between a solid or semi-solid surface, a liquid and the saturated vapour of said liquid. Different methods are applied to generate a three phase boundary, as e.g. the captive bubble method, where a bubble of saturated vapour of the used test liquid is captivated by the test surface. With respect to the claims in this invention, it is the advancing contact angle which characterizes the material surfaces.
Deactivation: Alteration of an active form or conformation to a less active form or conformation.
Density: Mass per volume (concentration) or per area (lateral density).
End group: Distal part of a macromolecule.
Excluded volume: Interaction between segments of solvated macromolecules or polymer chain(s) that are moving to occupy the same space.
Extracellular matrix (ECM): Meshwork synthesized by cells and composed of adhesive proteins such as glycoproteins, laminin, FN, interconnected collagen fibrils, hyaluronate and proteoglycans as structural and functional support of tissue cells.
Film: Synthetic material in the form of long, thin sheets.
Flexible: Capable of attaining many conformations, in contrast to rigid.
Flux: Measure of the flow of some quantity per unit area per unit time
Functionalization: Chemical derivatization changing structure, properties and/or function.
Grafting: Attaching at least one macromolecule comprising equal or different molecular units to a substratum through a chemical bond.
Head group: Proximal group, the group forming the link between a macromolecule and a substratum.
Hydrophilic polymer: Any polymer with a high surface energy where droplets of water spread readily.
Hydrophobic polymer: Any polymer with low surface energy where water forms prominent droplets on the surface.
Improved contact: Enhanced attachment and spreading of cells upon contact with non-biological supports.
Interface: Area or surface that represents the boundary between two separate phases of a chemical or physical process.
Ionic bond: Bond held together by coulombic interactions between differently charged moieties.
Latent: Present but not (yet) active.
Laterally structured monolayer: Monolayer formed of macromolecules interacting with neighboring molecules due to their inherent excluded volume, to spontaneously form a relatively ordered array of macromolecules, said monolayer is not crystalline and characterized by a water content of at least 50 percent.
Layer density: Mass per area (2D concentration).
Linker: Connects two moieties or groups or molecules with each other.
Macromolecule: Any molecule having a MW higher than 400 Da.
Membrane: Barrier between two phases and allowing transport via sorption/diffusion and/or through pores.
Permeability: Measure of the capability of a membrane to allow transport through said membrane.
Photo: Physical stimulus, here to initiate a chemical reaction.
Photo-reactive polymer: Polymer comprising one or more latently reactive groups.
Polymer: Molecule formed by the union of at least five identical monomers.
Pretreatment: The addition of functional groups including charged species and/or free radicals to a substratum and/or the conversion of one or more groups of a substratum to charged species and/or free radicals Receding contact angle: Contact angle when the liquid front is caused to recede over said solid.
Refractive index: Ratio of the phase velocity of electromagnetic radiation in a vacuum (or air) to that in a transparent medium.
Rigid: Essentially non-flexible.
Saturated substratum: Saturation of a substratum is attained, when the contact angle of said substratum contacted by a plurality of macromolecules can not be further reduced by adding further macromolecules to the surface of the substratum. More preferably, saturation of a substratum is attained, when no significant change of the contact angle can be achieved when said substratum is being contacted by a plurality of macromolecules.
Self-assembled monolayer: Monolayer formed on a substratum and comprising self-assembled (stacked or crystallized) components comprising a headgroup, said headgoup interacting favorably with the substratum, and an endgroup, said endgroup being orientated towards the solution. Said monolayer is characterized by a crystal-line, highly ordered structure and a very low water content or substantially no water content
Solvated: Molecule or material being in solution.
Synthetic material: Any material that is not of biological origin.
Substratum: Any chemical moiety to which macromolecules are capable of attaching.
Surface: Outer part of an object, here the biomaterial or its precursor.

The present invention teaches a new way of controlling cell adhesion and biocompatibility of polymer substratum surfaces associated therewith. The novel approach is based on a structuring of a hydrophobic substratum surface, preferably a hydrophobic polymer substratum, with a layer of macromolecules, preferably a monomolecular layer of flexible macromolecules, more preferably a monolayer of laterally patterned macromolecules contacted with said surface of said hydrophobic polymer substratum.

Non-biological materials that are relatively hydrophobic in nature or comprise a substratum comprising a hydrophobic polymer are water repellent, or have a limited wettability by water, and exhibit poor biocompatible properties. Proteins are known to adsorb abundantly to such hydrophobic surfaces and to denature upon contact. This denaturing (or unfolding) of adsorbing proteins is commonly held responsible for the poor biocompatible properties of these materials.

A non-biological material according to the present invention essentially does not alter the functionality of biological matter contacting the material. The contacting takes place at the interface between the non-biological material and the surrounding biological matter (like e.g. tissue and individual cells). Although such contacting events generally have significant effects on the biocompatible properties of a non-biological material, the biological matter contacting the biocompatible material according to the invention differentiates/proliferates as if present in a natural environment.

Accordingly, the biocompatible material according to the invention is capable of interacting with proteins/cells/tissue without essentially inducing e.g. protein denaturation; foreign body responses, inflammation, or cell death.

The response of essentially biological systems to the designed surfaces according to the invention is different from the response of such systems to the polymers of the prior art. Protein adsorption, antibody binding to adsorbed antigens as well as studies with fibroblasts, endothelial cells, keratinocytes, liver cells and others—i.e. biological materials well accepted as a general cellular model for tissue-biomaterial interaction —have been carried out in order to evaluate the ability of the novel biocompatible surfaces to support and/or improve the function of biological material brought in contact with it. The ability of cells to attach, spread and proliferate on various surfaces that had been modified according to the invention is of particular importance in that context. The production of an extracellular matrix is one of several key functions of fibroblasts and generally a characteristic feature of cells of the connective tissue type. Consequently, the ability of cells to attach to biomaterials according to the invention has been studied by microscopical investigations of extracellular matrix formation i) within the first hours of cell attachment, by means of fluorescently labeled FN, and ii) following long-term culture through direct detection of the synthesized FN matrix.

The studies revealed an improved cellular functionality as a function of e.g. the MW of the macromolecules attached to the polymer substratum, and the degree of surface functionalization. The results were measured by typical biocompatibility parameters such as e.g. cell adhesion and morphology, formation of focal adhesions points, the formation of an extracellular matrix, and the effect onto the long-term proliferation. All of the above is understood to contribute to the observed improved cellular functionality as defined herein above. In other words, the results clearly showed that polymer substratum surfaces modified according to the invention has a superior functionality. The functionality is superior when compared to both the original, unmodified polymer, and to the fully modified or “coated” surfaces of the prior art that are characterized by a comparatively high degree of surface functionalization.

The results described herein are strong indications that it is possible to further optimize the relationship that exists between adsorption of essentially biological material, the state, or conformation, or biologically active form of said adsorbed material, and the cellular behavior or functionality resulting from said adsorption.

Consequently, the invention makes it possible to determine empirically one or more optima of cellular functionality by means of a rational design approach that is readily controllable by any suitable state of the art physico-chemical surface analysis. Hence, the present invention achieves its objective by significantly improving state of the art methods of providing biomaterials, since the response of adsorbed cells and their biocompatibility can now be predetermined or at least designed quickly and economically by well-defined and readily adjustable state of the art physico-chemical and bioengineering parameters.

Materials that are capable of being processed according to the invention are those with at least suitable, if not superior physico-chemical properties for any given application, such as e.g. suitable or superior properties like transparency, refraction index, electrical conductivity, thermal stability, hydrolytic resistance, or membrane forming properties (ranging from, e.g. cell-culture dishes to membranes), but are currently less than adequate, if not entirely useless, for the attachment, growth and function of cells because of their undesirable physico-chemical surface properties.

The surface structures of the biomaterials to be processed in accordance with the invention may be porous structures with a stochastic or predetermined or controlled permeability (e.g. micro- or macro-porous flat-sheet or hollow-fibber membranes) that may be built up as a temporary or permanent support of cells described herein immediately below.

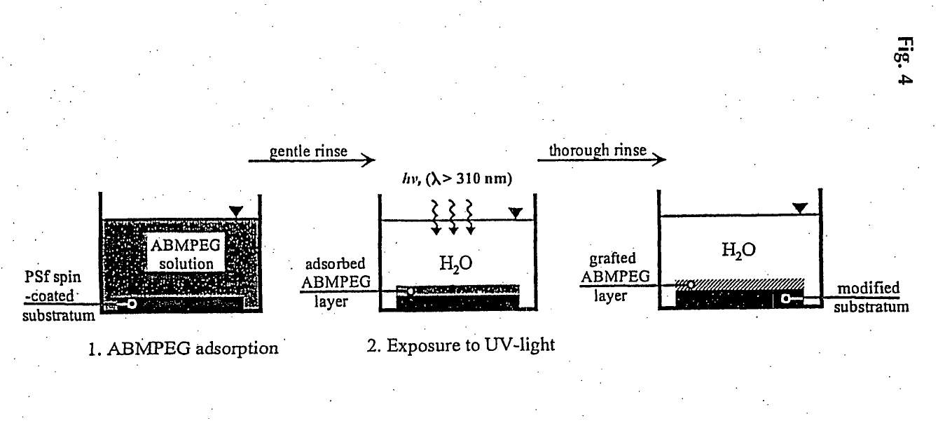

The two-step modification technique disclosed herein (see FIG. 4) preferably generates a covalently bound, patterned molecular monolayer. The structure or functionality of the layer may be designed or predetermined by synthesis of macromolecule conjugates and then in a first adsorptive step according to any given set of particular circumstances. By covalent grafting, a stable attachment (i.e. grafting) to the underlying polymeric material (basis polymer) is readily achieved. The control of the “design parameters” such as e.g. molecular structure of the amphiphilic macromolecule, the concentration and/or solvency of said macromolecule can be left to a person skilled in the art of manufacturing complex polymers.

MW and/or size of the amphiphil determines at least to some extent the molar density (i.e. macromolecules per surface area). An increased interaction between the amphiphilic macromolecule and the polymer substratum is likely to lead to an increased layer density. Likewise, a high concentration of amphiphilic macromolecules in the first step (see FIG. 4), or a decreased solvency of said amphiphilic macromolecules will also contribute to an increased layer density. Changes in solvency may be attainable through variations in e.g. salt concentration, pH, temperature or polarity of the solvent. Application of the amphiphiles by spray-coating and subsequent drying followed by ultra violet WV) or visible (Vis) irradiation can be alternative technologies. The person skilled in the art is familiar with the physical chemistry of polymers and macromolecules required in order to attain an altered layer density.

It is understood that when the biomaterial is a film, the polymer substratum is substantially inpenetratable to water, whereas the polymer substratum is porous, when the biocompatible material is a membrane.

The created lateral layer structure according to the invention is characterized by the amphiphil nature of the macromolecule and amphiphil-amphiphil intermolecular and intramolecular interactions. Strong repulsive interactions between the amphiphiles due to their inherent large excluded volumes lead to discretely adsorbed molecules capable of forming a laterally “self-assembled” structure.

Pretreatment of the Substratum According to the Invention

In one embodiment the present invention pertains to a material i) having a first contact angle, and ii) comprising a substratum having a second contact angle, said substratum i) being contacted by a plurality of soluble substances capable of forming a self-assembled monolayer comprising a macromolecule, and ii) having a: third contact angle when being contacted by a plurality of soluble substances capable of forming a self-assembled monolayer comprising a macromolecule.

The relation between the above contact angles as defined by the ratio between i) and ii), where
i) is the difference between a) the third contact angle of said monolayer, when no macromolecule is present, and b) said first contact angle, and
ii) is the difference between c) the third contact angle of said monolayer, when no macromolecule is present, and d) the contact angle of said self-assembled monolayer, when said monolayer is saturated by said macromolecules as defined herein, and
   wherein said ratio is preferably more than about −0.6 and less than about 0.6, such as less than 0.55, for example less than 0,50, such as less than 0.45, for example less than 0.40, such as less than 0.35, for example less than 0,30, such as less than 0.25, for example less than 0.20, such as less than 0.15, for example less than 0.12, such as less than 0.10, for example less than 0.08, such as less than 0.05.

The soluble substances are preferably selected from the group consisting of molecules capable of forming a self-assembled monolayer including low molecular weight chemical species such as e.g. branched or unbranched aliphatic carbon compounds having a chain length of from 1 to about 20 carbon atoms.

Additionally preferred species capable of forming a self-assembled monolayer may be selected from the group of monomers, or mixture of monomers, consisting of C₄-C₁₈ alkylacrylates, and the respective amides, C₄-C₁₈ methacrylates, and the respective amides, 2-C₁-C₁₀ alkylcyanoacrylate and diisocyanate, 2-ethylcyanoacrylate and toluen 2,4-diisocyanate, acrylic acid, methyl acrylate, 2-hydroxyethyl-acrylate, N-ethyl-2methyl allylamine, glycidyl methacrylate, diallylamine, and/or other vinyl group containing monomers.

Another group of materials according to the invention capable of being pretreated are materials comprising a substratum, said substratum being contactable with a macromolecule, said material further comprising at least one macromolecule,
said material having a first contact angle a,
said substratum having a second contact angle b₀ when not contacted by a macromolecule, and another second contact angle bₛₐₜ, when said substratum is saturated by said macromolecules as defined herein,
wherein the relation between said contact angles is as defined by the ratio R,
   R=(b₀−a)/(b₀−bₛₐₜ)
   and wherein the numerical value of R is in the interval from and including 0 to less than 0.6, such as less than 0.55, for example less than 0,50, such as less than 0.45, for example less than 0.40, such as less than 0.35, for example less than 0,30, such as less than 0.25, for example less than 0.20, such as less than 0.15, for example less than 0.12, such as less than 0.10, for example less than 0.08, such as less than 0.05.

The second contact angle b₀ refers to the second contact angle of a substratum, which may or may not be pretreated. The substratum may for example be pretreated by any of the methods described herein below.

Yet another group of materials according to the invention capable of being pretreated are materials comprising a substratum, said substratum being contactable with a macromolecule, said material farther comprising at least one macromolecule,
said material having a first contact angle a,
said substratum having a second contact angle b₀ when not contacted by a macromolecule,
said contact angle a being substantially identical to said contact angle b₀.

When the above stated materials are pretreated or modified according to the invention, they are preferably being contacted by and/or operably linked to a charged species or a charged chemical group including a hydrophilic compound. Accordingly, the present invention provides a method of pretreatment of a substratum according to the invention having a second contact angle, wherein said pretreatment is preceding a further method step of contacting the substratum with a macromolecule, said contacting generating a biocompatible material as defined herein and having a first contact angle.

Well known techniques known as corona treatment or plasma treatment can be used to perform the pretreatment of the substrata and polymeric materials pertaining to the present invention. Furthermore, a variety of other pretreatments may be employed with the present invention, for example pretreatments that involves the formation of free radicals, such as irradiation techniques including for example electron beam treatment or sonochemical techniques or any wet chemical treatment for surface activation including but not limited to treatment with peroxides.

Corona treatment is described e.g. by Podhajny, R. M.; (1988): Corona treatment of polymeric films. J. Plast. Film Sheeting. 4: 177-88, and by Sun, C., D. Zhang, et al. (1999). Corona treatment of polyolefin films—A review. Adv. Polym. Technol. 18: 171-180, both of which are incorporated herein by reference. Corona discharge introduces polar groups into the polymeric surfaces and, as a consequence, improves the surface energy, wettability, and adhesion characteristics. The main chem. mechanism of corona treatment is oxidation.

Plasma treatment is described by among others Oehr, C. and H. Brunner (2000). Surface treatment of polymers with glow discharges. Vak. Forsch. Prax. 12: 3540, and Oehr, C., B. Janocha, et al. (2000). Plasma treatment of polymers for medical and biological applications. Vak. Forsch: Prax. 12: 313-317, both of which are incorporated herein by reference. A low-pressure plasma treatment of polymer substrata according to the invention results in improved properties and functionalities such as e.g. sterilization by, plasma treatment, facilitating in the controlling of the protein adsorption, improving cell growth and proliferation.

Corona treatment as used herein, refers to electrical discharges that occur at substantially atmospheric pressure. However, other types of electrical discharges such as sub-, atmospheric and vacuum-pressure electrical discharges or processes; as well as subatmospheric and atmospheric “glow” discharges (as described in European Patent Publication No. 603784) are not normally associated with the term “corona treatment”, but sub-atmospheric and vacuum-pressure electrical discharges or processes, as well as subatmospheric and atmospheric “glow” discharges may also be used and thus fall under the term “pretreatment” as used herein.

One purpose of performing a pretreatment including a corona treatment (“coronapriming”) or a plasma treatment, of a polymer surface like the substrata according to the present invention is to improve the wettability (reduce the hydrophobic nature of the substratum) of the surface of the polymer or the substratum. The substratum will generally acquire an lower advancing contact angle following pretreatment. It is thus possible to treat hydrophobic substrata that might not otherwise have been suitable for contacting with a macromolecule according to the method disclosed in the present invention. Initial pretreatment thus widens the kind of substrata capable of being modified according to the present invention. This in turn increases the commercial applicability of the present invention.

As pretreatment in general serves to increase wettability it also improves the interaction of the surface of substrata with macromolecules used to create the surface modification. This is particularly important in connection with the present invention as it provides a means for broadening the kind of substrata that can be subjected to subsequent surface modification according to the invention by means of contacting a predetermined substratum with a macromolecule essentially without altering the contact angle of the substratum.

Corona priming of substrata such as e.g. polymer films in air to increase wettability in order to increase macromolecule interactions can be accomplished by any number of well-known techniques. Importantly, air corona priming is typically performed in the presence of ambient atmospheric gases (i.e., nitrogen and oxygen and trace gases) at atmospheric pressure. Corona processes are fast and cheap, and generally susceptible of application to in-line industrial processes including subatmospheric and vacuum-pressure processes.

The corona pretreatment process of the present invention provides an effective and efficient initial surface treatment of e.g. polymer films that produces significant and advantageous modification to polymer surfaces; it is less expensive and time consuming than sub-atmospheric processes that require complex vacuum producing apparatus. Because of its-low cost and efficiency, it is readily susceptible to application in an in-line industrial process, which is particularly important in the processing of polymer films that are supplied in roll form. Moreover, the low cost of nitrogen as a major atmosphere component makes the process of the present invention attractive for application as a large-scale industrial process.

The corona pretreatment optionally utilized in the present invention may be characterized in terms of a “normalized energy” which is calculated from the net power and the velocity of the polymer film being treated in the corona treatment system, according to the following formula:
normalized energy=P/wv
where P is the net power (in Watts), w is the corona treatment electrode width (in cm), and v is the film velocity (in cm/s). Typical units for normalized energy are Joules/square centimeter. In preferred embodiments of the present invention, the corona discharge is characterized by having a normalized energy of between about 0.1 and about 100 Joules per square centimeter, preferably from about 1 to less than 80 Joules per square centimeter, more preferably from about 1 to less than 50 Joules per square centimeter, and even more preferably between about 1 and about 20 Joules per square centimeter.

U.S. Pat. No. 5,972,176 incorporated herein by reference discloses one method for pretreating a substratum according to the present invention by exposing the substratum to a corona discharge at substantially atmospheric pressure in an atmosphere comprising a major proportion of nitrogen gas and about 0.01 to about 10 volume percent hydrogen. However, other types of corona pretreatment methods, may also be applied in accordance with the present invention.

WO 98/00457 incorporated herein by reference discloses another suitable pretreatment method for modifying the surface of a polymer substratum according to the present invention. Accordingly, pretreatment may be carried out in accordance with the present invention by a) generating radicals on the substratum surface by corona treatment, by subjecting the substratum to a gas plasma, or by subjecting it to a suitable radiation source including UV light, and b) treating the surface with a vapour of a suitable monomer or a mixture of monomers.

The monomer or mixture of monomers may comprise e.g. one or more of 2-C₁-C₁₀ alkylcyanoacrylate and diisocyanate, one or more of 2-ethylcyanoacrylate and toluen 2,4-diisocyanate, one or more of acrylic acid, methyl acrylate, 2-hydroxyethyl-acrylate, N-ethyl-2methyl allylamine, glycidyl methacrylate, diallylamine, and/or other vinyl group containing monomers.

The polymer substratum capable of being subjected to a pretreatment involving a) generating radicals on the substratum surface by corona treatment, by subjecting the substratum to a gas plasma, or by subjecting it to a suitable radiation source including UV light, and b) treating the surface with a vapour of a suitable monomer or a mixture of monomers, can be of any polymer material provided that free radicals are created on the surface of the material when it is subjected to corona treatment, gas plasma and/or treated with UV light.

When the generation of radicals on the substrate surface is obtained by subjecting the substrate to UV light, the wavelength and the intensity of the UV light are selected depending on the constitution of polymer. A skilled person can by use of ordinary techniques optimise the method by selecting wavelength and intensity of the UV light as well as selecting the time of radiation. The time of radiation should naturally be sufficiently long to create the radicals on the surface. On the other hand, the time of radiation should not be too long, as this might result in degradation of the substrate.

The generation of radicals on the substrate surface is preferably obtained by subjecting the substrate to a gas plasma. The plasma can be generated by any known methods, but preferably the gas plasma is generated by excitation of a gas in a direct current (DC), audio frequency (AF), radio frequency (RF) or microwave (MW) generated electric field. Most preferably the gas plasma is generated by excitation of a gas in a direct current (DC) or by exitation using radio frequency (RF).

The intensity of the used gas plasma should preferably have a level ensuring creation of radicals in the polymer surface. If the level is too high, this may result in severe damage of the bulk-polymer (depolymerization). Hence, the power level of the plasma should be optimized so that surface radicals are created, but no serious damage is made to the bulk.

The gas plasma can for example be any inert gas or mixtures thereof, preferably a gas selected between He, Ne, Ar and Kr. By the term “inert gas” is meant a gas that does not react chemically with the polymer surface. Furthermore, for example oxygen and/or nitrogen plasma may be applicable.

In one embodiment of the present invention the substratum is pretreated by a method that involves formation of free radicals. It has been found that initiation of free radicals in a polymeric material by electron beam or other free radical initiating treatment enhances binding of a bioactive reagent for example a ligand to the polymeric material. For example polystyrene surfaces exposed to electron beam activation demonstrated markedly increased affinity for selected ligands.

Electron beam processing is described in detail in e.g. Stern, M. (2001). Electron-beam processing of thermoplastics: A review. Int. SAMPE Symp. Exhib. 46: 2536-2549, which is incorporated herein by reference. E-beam processing (EBP) is used for improving thermal, chemical, barrier, impact, wear, and other properties of polymer substrata according to the invention, extending their utility to demanding applications typically dominated by higher-cost engineered materials.

For example the substratum may be pretreated by treatments such as admixture or by chemical grafting. Techniques used for grafting include steps for free radical initiation, for example, by irradiation techniques including, but not limited to, electron beam treatment or sonochemical techniques.

Selected Polymer Substrata Capable of being Modified

Synthetic polymer substrata according to the present invention can preferably be selected from the group of substrata consisting of any bioerodible polymer and any non-erodible polymer including any pretreated bioerodible polymer and any pretreated non-erodible polymer, wherein the term pretreated preferably denotes any pretreatment method described herein.

Accordingly, one group of substrata according to the present invention consists of polymers, including pretreated polymers, such as poly(lactide) (PLA), poly(glycolic acid) (PGA), poly(lactide-co-glycolide) (PLGA), poly(caprolactone), polycarbonates, polyamides, polyanhydrides, polyamino acids, polyortho esters, polyacetals, polycyanoacrylates and degradable polyurethanes.

Another group of substrata according to the present invention consists of polymers, including pretreated polymers, such as polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolefins, polyethylene oxide, polyvinyl alcohol, teflon.RTM., and nylon.

Other suitable polymer substrata according to the present invention are silicon rubbers, and any thermoplastic polymer including any polyolefin, including any pretreated silicon rubber, any pretreated thermoplastic polymer including any pretreated polyolefin.

The thermoplastic polymer may be of any type of heat processable polymer preferably comprising an olefinic component. The polymeric material is very conveniently an olefin polymer. The olefin polymer, which term is used herein to include both homopolymers and copolymers containing at least 50% by weight of one, or more, olefin monomers, is a polymer of an olefin monomer which typically contains not more than ten carbon atoms, and preferably of the monomers ethylene or propylene. Thus, the olefin polymer may be any ethylene homopolymer (polyethylene), copolymer or terpolymer, particularly high density polethylene or linear low density polyethylene which is a copolymer of ethylene with a higher alfaolefin monomer such as butene, hexene octeneor 4-methylpentene. Other ethylene polymers are the copolymers of ethylene and a monomer, for example an ethylene-vinyl acetate copolymer typically one containing 10 to 40% by weight of vinyl acetate. Alternatively, the olefin polymer may be a propylene homopolymer or copolymer, for example a random copolymer of propylene with up to 8% by weight, relative to the polymer, of ethylene, or a sequential polymer obtained by polymerising propylene in the essential absence of other monomers and thereafter copolymerising a mixture of ethylene and propylene to give a polymer containing from 5 to 30% by weight of ethylene.

Other examples of substrata which may be used with the present invention are polysiloxane, polystyrene-butadiene co-polymers, tetrafluororethylene, polycarbonate, polyvinylpyrrolidone, dextrans, polyethylene terephtalate, and polysulfone. Furthermore, a hydrophilic charged macromolecules are applicable to use with the present invention. Examples of hydrophilic charged macromolecules are polyacrylic acid (PAA), polysaccharides, such as hyauronic acid (HA) and alginate acid (AA) as well as a large number of other polysaccharides.

Preferred polymers are homo- and copolymers of linear low density polyethylene (LLDPE), Low density polyethylene (LDPE), High density polyethylene (HDPE), Ethylene/vinylacetate (EVA), Ethylene-methyl-acrylate (EMA), Ethylene-acrylic-acid (EAA), Ethylene-butyl-acrylate (EBA), Ethylene-ethyl-acrylate (EEA), Polypropylene (PP), Ethylene-propylene copolymer (EPM), and Ethylene-propylene-diene terpolymer (EPDM).

The thermoplastic polymer in one embodiment is preferably selected from the group consisting of polytetra-fluoroethylene (PTFE), tetra-fluoroethylenehexa-fluoropropylene-copolymer (FEP), polyvinyl difluoride (PVDF), polyamides, such as e.g. nylon 6.6 and nylon 11, polyvinyl-chloride (PVC), polystyrene, and polyurethane.

The polyolefin is preferably selected from the group consisting of polyethylene (PE), high density polyethylene (HDPE), low density polyethylene (LDPE), polypropylene (PP) and poly(4-methyl-i-pentene) (PMP).

For certain medical applications 7 be it in vitro or in vivo—it is preferred that the substratum is selected from the group consisting of moderately to highly hydrophobic polymer substrata, such as, but not limited to, polysulfon and derivatives thereof, polyethersulfon and derivatives thereof, sulfonated polysulfon and derivaties thereof polyacrylonitrile and derivatives thereof, polymethylmethacrylate and derivatives thereof, polycarbonate and derivatives thereof, and polyamide and derivatives thereof. Derivatives include substrata that has been pretreated according to the invention. Derivatives shall also denote any substratum having a backbone structure as described herein above.

The polymer substratum in one embodiment is in the form of a film, a sheet, a pipe, a rod, a porous or non-porous body, a fabric, a nonwoven fabric, a fibre or a thread.

The polymer substratum may be produced by injection moulding. A particularly preferred polymer is one having a molecular weight which is appropriate for a material which can be used for the production of shaped articles by an injection moulding or extrusion process. Thus, suitable olefin polymers, such as LLDPE, LDPE, HDPE, EVA, EEA, EMA, EAA, EDA and PP are those having a melt flow index, measured according to ASTM Test Method 1238-79 using a 2.16 kg weight at a temperature of 230° C., which is in the range from 0.5 g/10 minutes up to 50 g/10 minutes preferably from 1.0 g/10 minutes up to 30 g/10 minutes. The biocompatible material according to the present invention has many applications as an interface between biological and non-biological materials both in vitro and in vivo. The following paragraphs highlight a few selected areas wherein the present invention is capable of replacing state of the art products that do not provide the superior technical effects that are achieved by the present invention

Biocompatible Materials and Methods According to the Invention for Culturing Adhesion-Dependent Cells in Vitro

The biocompatible material according to the invention is well suited for culturing adhesion-dependent cells in e.g. low serum or serum-depleted media. The culture of adhesion dependent cells normally requires the presence of so-called attachment factors on the substratum. These adhesion factors involve serum components, such as fibronectin and vitronectin that readily adsorb onto the substratum of tissue culture plastic ware. Cells interact with these adsorbed adhesive proteins via specific cell-surface receptors called integrins.

Via the interaction with integrins these attachment factors provide not only the possibility for cells to anchor physically on the substratum, the attachment factors also deliver appropriate physiological signals into the cells. The integrins transduce signals that influence a broad range of cellular processes, including migration, spreading proliferation and transformation. Without appropriate signalling, a programmed cell death called apoptosis may be induced. The presence of adsorbed adhesive proteins on the substratum may therefore decide the failure or success of in vitro culture of cells.

The presence of serum components in media for cell culture is currently considered to be essential for a successful culture of cells. However, the composition and quality of serum preparations is dependent on the donor herd, and large variations can be observed from batch to batch. It is an additional problem that serum may contain several components such as e.g. albumin whose presence may inhibit the attachment of cells. Also, the addition of serum increases the risk for transmission of infectious agents, such as viruses, prions, etc.

One way of trying to solve these problems has been the introduction of defined supplementa containing growth factors and other factors as a replacement of serum in cell culture media. One problem associated with the serum-depleted media is the absence or low concentration of attachment factors. This results in a reduced adhesion of cells, and subsequent a lack of adequate proliferation. Even when adhesion factors are present in serum supplement media, their concentration is often too low, or their conformation is changed upon adsorption because of the absence of structure stabilising proteins.

Plastic culture ware delivered from companies like SIGMA or ICN tries to overcome the difficulties with these preparations. Both companies offer tissue culture materials having surfaces treated with recombinant proteins containing multiple RGD binding sites. These substrata aims to provide a good adhesion for a variety of cells and enhance functionalities such as e.g. morphology and metabolism.

The present invention offers a superiour alternative to the state of the art products by providing a simple chemical technology that would allow the adsorption of attachment factors in a physiological active conformation under low serum conditions, or circumstances wherein fibronectin or other attachment factors are used as a supplement in the absence of serum.

As many types of cells are able to secrete their own adhesive proteins, the present invention provides conditions under which the conformation of the adsorbed proteins is stabilised and conserved as it would have been under natural conditions. The present invention also avoids using artificial proteins and complicated processes involving binding chemistry and sterilisation.

Culturing adhesion-dependent cells in low serum or serum-depleted media represents a focus area of the present invention. The technology according to the invention can be used to produce tissue culture plastic ware for culturing adhesion dependent cells including vertebrate cells including human and/or animal cells under low serum or serum absent conditions.

Selected embodiments of the aspect of the invention directed to tissue culture plastic ware for culturing adhesion dependent cells is described herein below.

In one embodiment of the present invention there is provided an improved biocompatible material as disclosed herein, including a polystyrene surface or a polycarbonate surface, for tissue culturing, and a method for producing an improved tissue culture-treated plastic surfaces, such as, for example, polystyrene assay plates.

Methods and materials for the facilitation of high-protein-binding capability on tissue culture-treated (“TC”) plastic assay plates are well known in the art and described in e.g. U.S. Pat. No. 6,040,182. Such methods and materials include the use of appropriate coating buffer (“CB”) components to facilitate a high protein-binding capability. Of particular interest within the area of protein immobilization is the immobilization of antibody molecules due to their ease of preparation and high diversity of binding specificity.

Historically, nearly all efforts to achieve efficient protein: binding to, for example, polystyrene surfaces by passive, noncovalent adsorption have employed the very basic carbonate or mixed bicarbonate/carbonate buffers at pH 9.6 as CB. See, for example, Butler, J. E. et al., J. Immunological Methods, 150: 77-90 (1992). This type of buffer has provided good results when used with classical first-generation hydrophobic assay plates or second-generation hydrophilic assay plates, but not when used with TC-treated plates.

If an alternative buffer is employed, it is most commonly phosphate buffer, phosphate-buffered saline or tris buffer at pH of 7.0-8.0. In general, the use of organic CB's other than tris in protein immobilization has been ignored. Certainly, the use of organic CB's in conjunction with TC-treated plates for the purpose of efficient protein immobilization has been ignored, since TC-treated plates have not generally been used to perform heterogeneous immunoassays.

The phenomenon of passive noncovalent binding of protein species to polystyrene is well-known as a practical means of immobilization of assay components, where such immobilization is useful to allow rapid, simple and efficient “bound vs. free” separation(s) to be performed in support of specific detection of particular analyte(s). In general, solid phase binding-based (i.e., heterogeneous) assay formats employ immobilized species (“Capture Antibody” [CAb], if it is an antibody [Ab]) as the starting point for building an appropriate signal-mediating specific binding cascade on the solid support surface. This binding cascade is engineered such that the presence of analyte in the test sample is either: a) required in order to complete the binding cascade, as a necessary prerequisite before signal production may occur (“direct assay” format); or, b) required in order to inhibit binding of a detectable “conjugate” species, which consists of a chemically derivatized version of the analyte (indirect or “inhibition assay” format). The label/reporter species of the conjugate must include functionality which supports detection by appropriate means (e.g., bearing radioisotope, fluorophore or enzyme “label” and/or “reporter” species), while retaining essential binding motifs of the analyte fragment which are required for specific interaction with the CAb species and/or other binding partner(s). With the direct assay format, signal produced in the assay is proportional to the amount of analyte present in the sample. Alternatively in the inhibition assay format, the signal produced by the binding cascade is inversely proportional to the analyte concentration, due to the competition for a limited number of binding cascade sites between analyte (variable amount; detection not facilitated) and conjugate (fixed amount added per tube or well; detectable by design).

In many cases, the protein to be immobilized consists of an antibody (such as, for example, immunoglobulin G [IgG]) which exhibits specific binding with high affinity to the analyte of interest. In other cases, non-antibody proteins which exhibit specific binding capability (e.g., streptavidin, which is known to bind biotin with extremely high specificity and affinity) may be immobilized. In general, to be of practical utility, such immobilization needs to exhibit the following properties: a) high efficiency of protein binding (high level of polystyrene surface coverage [about 100-400 ng/cm²], ideally with a high fraction of input protein bound [e.g., 10-99%]), b) high stability of immobilized protein with respect to [undesired] wash-off during bound vs. free” separation (“wash”) steps; c) high retention of native conformation and biological activity; as well as d) high, substantially complete retention of binding properties of the immobilized protein vs. its solution-phase counterpart (in terms of binding affinity, binding specificity and kinetic parameters). Finally, the immobilization process must not introduce conformational or other changes in the CAb or other immobilized species which result in “non-specific binding interactions” (NSB) with other assay reagents and/or sample components. Since in general a large portion of the immobilized CAb (typically about 90% for polyclonal antibodies [pAb's], 90-99% for monoclonal antibodies [mAb's]) or other first binding partner is denatured in the course of immobilization, the latter concern regarding possible NSB is not a trivial one.

The literature, such as Butler et al., supra, indicates that passive adsorption of proteins on polystyrene is an extremely complex, incompletely understood and often unpredictable phenomenon. Historically, assay plate manufacturers have dealt with this serendipitous aspect of the application arena by providing a family of assay plate products which provide a range of polystyrene surface characteristics, from hydrophobic to hydrophilic in character. By screening a variety of tailored surface chemistries for their ability to support efficient immobilization of the desired CAb or other first binding partners an appropriate solid phase surface chemistry can be selected which allows adequate assay performance to be demonstrated. While it is generally understood that varying the pH of the “Coating Buffer” (CB) used (i.e., the CAb diluent) can modulate the binding obtained, for theoretical reasons primarily related to the presence of a “Linear Binding Region” (LBR) in “% bound” plots of CAb binding as a function of amount of input CAb when using pH 9.6 buffer only as the CB, the vast majority of passive adsorption experiments have employed the carbonate or carbonate/bicarbonate buffer systems at pH 9.6 as “standard CB”. The rationale offered for this observation was that the efficiency of passive adsorption is dependent on aggregation of the protein to be immobilized, and that such aggregation was disfavored at pH values more acidic than that of the “standard pH 9.6 CB” when Ab concentration is low. Accordingly, most skilled practitioners of the art employ pH 9.6 buffer(s) (or variants thereof) as CB exclusively, and simply test a large number of possible assay plate types in conjunction with the “standard pH 9.6 CB” to optimize CAb or first binding partner immobilization in their assay development efforts.

It is important to note that early assay plates were either underivatized or lightly derivatized (e.g., gamma irradiated) polystyrene materials of predominately hydrophobic nature. Later developments in “high protein binding” plates provided considerably more hydrophilic surfaces, e.g. by W-irradiation of the polystyrene surface. The hydrophilic character of the second-generation assay plates proved to be superior in most cases of protein immobilization, since in general proteins contain a significant density of polar functional groups on their surface. However even using these more polar surfaces, it was evidently still advantageous to work at basic pH to avoid excessive charge density on the protein surface. At pH 9.6, the major difference relative to physiological pH or other more acidic conditions is that free amino groups of the protein are typically partially or completely deprotonated (in: free base form, thus neutral with no charge) instead of fully protonated (in positively charged, ammonium ion form).

In parallel to the above developments, a different class of “assay plate” was developed for the purpose of supporting mammalian cell attachment and growth (“tissue culture” [TC] plates). TC plates are prepared using a high energy plasma treatment process under oxidative conditions, either performed under partial vacuum as is done to make Falcon.RTM. standard TC and Primaria.RTM. TC plates, or alternatively at atmospheric pressure (corona discharge process). These TC plates exhibit a high degree of surface oxidation, and inretrospect it appears that there may be a higher ratio of carboxylate groups present vs. hydroxyl groups, than is the case with classical high protein binding assay plates.

As oxidation of polystyrene generates a negativly charged surface, attachment of macromolecules according to the invention can conceivably also be carried out by other binding mechanisms, such as e.g. covalent binding of e.g. PEG to a positively charged polyethylene imine resulting in a Coulomb type of binding to the surface. Since proteins adsorb also on charged substrata there is a driving force for protein adsorption connected with the stabilsing effect of surrounding PEG.

Some practitioners do carry out binding based) homogeneous assays in TC plates due to the superior wettability of TC-treated polystyrene plates. Also, some assay developers may indeed have conducted heterogeneous assays with TC-treated plates (e.g., inhibition assays where a high amount of immobilized CAb is not required or desirable). However, the prior literature, including the teachings contained in U.S. Pat. No. 6,040,182, has not taught knowledgeable practitioners of the art how to effectively employ TC-treated plates as high-protein-binding-capable assay plates on a par in performance with classical high-protein-binding assay plates.

This is accomplished by the biocompaticle materials and methods for their manufacture pertaining to the present invention.

The methods of the present invention enables the use of TC-treated plastics, such as, for example, polystyrene, assay plates in heterogeneous immunoassay formats by disclosing a biocompatible material comprising a substratum contacted by at least one macromolecule,
said material having a first advancing contact angle a,
said substratum having, a second advancing contact angle b₀ when not contacted by a macromolecule, and another second advancing contact angle bₛₐₜ, when said substratum is saturated by said macromolecules,
wherein said advancing contact angles are measured using water and air saturated by water vapour,
wherein bₛₐₜ essentially does not change when the substratum is contacted by further macromolecules by means of a chemical bond,
wherein the relation between said advancing contact angles is as defined by the ratio R,
   R=(b₀−a)/(b₀−bₛₐₜ)
   and wherein the numerical value of R is in the interval from 0 to less than 0.4.

The material according to the present invention may also be characterised as a material comprising a substratum, wherein the material is generated by modifying the substratum by contacting the substratum with a macromolecule,
wherein said substratum is contactable with a macromolecule,
wherein said material further comprises, at least one macromolecule,
wherein said material has a first contact angle a,
wherein said substratum has a second contact angle b₀ when not contacted by a macromolecule, and
wherein said contact angle a is substantially identical to said contact angle b₀.

The material is capable of being produced by a high degree of reproducibility, and this is turn leads to products having a much reduced variability in quality, a problem associated with many state of the art biocompatible materials. The lack of such variability should promote more reproducible immobilization, and thus improve overall assay reproducibility (i.e., lower the observed coefficient of variation [“% C.V.”] values).

Furthermore, the biocompatible materials in the form of a substratum being modified by a macromolecule being bound thereto essentially without changing the contact angle of the substratum makes it significantly less likely that CAb or capture protein denaturation should occur during or after immobilization.

Method for Treatment of Infertility

In another embodiment of the present invention there is provided a method for treatment of infertility, a method for the improvement of implantation rates after in vitro fertilization (IVF).

Human in vitro fertilization is surprisingly unsuccessful. The overall birth rate per IVF treatment cycle is approximately 0.14% in USA (Medical Research International Society for Assisted Reproductive Technology [SART], The American Fertility Society [1992]. Fertil Steril 5:15), and 12.5% in UK (The Human Fertilization and Embryology Authority. Annual Report, London 1992).

Success is greater when more than one embryo is transferred simultaneously. However, simultaneous transfer of multiple embryos increases the incidence of multiple pregnancy and the possibility of miscarriage and prematurity. The reasons for the low pregnancy rates after IVF are still not completely understood. The quality of both the embryo and the uterine environment affects success. Generally, there is a high rate of spontaneous early abortion in fertile cycles in women. After natural conception, possibly as many as 50-60% of very early pregnancies are lost (Winston M L, Handyside A H [1993], New challenges in human in vitro fertilization. Science 260:932-935). This may be due to both conceptus abnormalities and dysynchrony between embryo and endometrium at the time of embryo transfer.

Most losses may be due to abnormalities of the conceptus or the still inappropriate culture conditions, since the success of embryo transfer after IVF decreases as the time after insemination increases (Winston M L, Handyside A H [1993], New challenges in human in vitro fertilization. Science 260:932-935).

To overcome possible deficiencies in culture media, transfer of oocytes (gamete intrafallopian transfer—GIFT) or zygotes directly to the fallopian tube (zygote intrafallopian transfer—ZIFT) has been performed in women with intact oviducts. However, these attempts only slightly increased the fertility and birth rates after IVF (Edwards R G [1995] Clinical approaches to increasing uterine receptivity during human implantation. Hum Reprod 10, Suppl 3:60-67).

It is an object of the invention to provide a method for the improvement of implantation rates after IVF comprising the steps of culturing an embryo, oocyte or zygote, to be implanted in a suitable serum or substrate in a container comprising a material according to the present invention comprising a substratum, preferably polystyrene including pretreated polystyrene, and implanting said embryo, oocyte or zygote in an endometrial environment of a female body.

It is another object of the invention to provide a method for treatment and/or prevention of infertility or early pregnancy loss comprising the steps of culturing an embryo, oocyte or zygote, to be implanted in a suitable serum or substrate in a container comprising a material according to the present invention comprising a substratum preferably polystyrene including pretreated polystyrene, and implanting said embryo, oocyte or zygote in an endometrial environment of a female body.

It is a further object of the present invention to provide a container comprising a material according to the invention capable of mimicking an endometrial environment of a female uterus. The container is in one embodiment used in conjunction with the above mentioned methods for the improvement of implantation rates after IVF, and methods for treatment and/or prevention of infertility or early pregnancy loss.

Over the past decade, investigators have come to recognize the importance of the extracellular matrix (ECM) in directing the growth, differentiation and function of the overlying epithelium. Getzenberg et al., “The Tissue Matrix: Cell Dynamics and Hormone Action”, Endocrine Rev., 11:399-417 (1990). The interaction between cell and extracellular matrix (or substratum) is mediated by several classes of cell adhesion molecules, one of the most important being the integrins. Albelda et al., “Integrins and Other Cell Adhesion Molecules”, FESEB J., 4:2868-2880 (1990). Buck et al., “Integrin, a Transmembrane Glycoprotein Complex Mediating Cell-Substratum Adhesion”, J. Cell Sci. Suppl., 8:231-250 (1987). This diverse family of glycoprotein receptors is expressed on the cell membrane as heterodimeric .alpha. and .beta. subunits and is involved in both cell-cell and cell-substratum adhesion. Specific recognition and binding of extracellular matrix (ECM) components such as fibronectin (N), lamin (LM) and collagen (Col) transmit information to the cytoskeletal structure, an interaction which may have major roles in promoting hormone responsiveness and genomic activation. Burridge et al., “Focal Adhesions: Transmembrane Junctions Between the Extracellular Matrix and the Cytoskeleton”, Ann. Rev. Cell. Biol 4: 487-525 (1988) and Getzenberg et al. supra.

Although extensive information exists about specific integrin proteins, for example, Hemler, M. E. “IVLA Proteins in the Integrin Family: Structures, Functions and Their Role on Leukocytes”, Annu. Rev. Immunol: 365-400 (1990), little is known concerning the distribution of these receptors in the female reproductive tract. In the uterus, the endometrium, composed of glandular epithelium and associated mesenchyme (stroma), maintains complex temporal and spatial functions in response to the cyclic hormonal milieu. The search for morphological or biochemical markers for uterine receptivity has been unsuccessful to date as reported by Rogers and Murphy, “Uterine Receptivity for Implantation: Human Studies”, in Blastocyst Implantation, Yoshinaga, K. ed, Serono Symposia, pp. 231-238 (1989). Once such markers are identified, their role in endometrial phenomena including embryo implantation, fertility, contraception and endometrial maturation and receptivity can likely also be identified. As some integrins appear to meet the criteria for markers of receptivity there is a great need for using such integrins and other morphological or biochemical markers for uterine receptivity in the creation of an in vitro environment mimicking an in vivo endometrial environment, in particularly with respect to the integrin cell adhesion molecules that are present in the endometrium.

In one particularly preferred embodiment there is provided a method for in vitro fertilization comprising the step of culturing an embryo in a container comprising a material according to the invention comprising a beta-3 subunit of an endometrial integrin, fertilizing an egg in vitro, and introducing the zygote into a serum comprised in the container. Accordingly, contraceptive and diagnostic kits are also contemplated in this aspect of the present invention.

The present invention is also directed to methods of in vitro fertilization. Once a suitable endometrial environment is detected in an animal selected for pregnancy, such as an environment comprising a beta-3 subunit of an integrin, a fertilizable egg (or eggs) from the same or different animal could be replaced into the uterus to establish pregnancy. The egg and appropriate sperm are combined to produce a zygote in vitro. For purposes of the invention, in vitro fertilization may take place in a petri dish or a test tube comprising a material according to the present invention, preferably, but not limited to polystyrene, or the like. In addition, in vitro fertilization may also refer to independently adding eggs and sperm to the fallopian tubes such that the zygote is formed therein. In any event, the zygote is introduced to the uterus of the animal selected for pregnancy and monitored for implantation into the endometrium of the uterine wall.

In another aspect of the present invention there is provided a method for increasing the fertilization potential of oocytes comprising culturing oocytes in vitro in a suitable serum or substrate comprised in a contained such as e.g. a petri dish or a test tube comprising a material according to the present invention, optionally culturing in vitro with an effective amount of inhibin, activin, or a combination of inhibin and activin as disclosed in U.S. Pat. No. 5,693,534. Preferably the material comprises a substratum in the form of polystyrene or pretreated polystyrene. After the culturing step, the oocytes can be fertilized. The oocytes are optionally suitably cryopreserved and thawed before the culturing step.

Inhibin and activin are members of a family of growth and differentiation factors. The prototype of this family is transforming growth factor-beta (TGF-.beta). Derynck et al., Nature, 316: 701-705 (1985); Ying et al., Biochem. Biophys. Res. Commun., 135: 950-956 (1986).

Inhibin is a glycoprotein produced by diverse tissues, including the gonads, pituitary, brain, bone marrow, placenta, and adrenal gland. It was initially identified by its ability to inhibit the secretion of follicle stimulating hormone (FSH) by the pituitary. De Jong and Sharpe, Nature, 263: 71-72 (1976); Schwartz and Channing, Proc. Natl. Acad. Sci. USA, 74: 5721-5724 (1977).

After the identification of inhibin, activin was shown to exist in follicular fluid as a naturally occurring substance. Activin was found to be capable of stimulating FSH release by rat anterior pituitary cells. Vale et al., Nature, 321: 776-779 (1986); Ling et al., Nature, 321: 779-782 (1986); DePablo et al., Proc. Soc. Exp. Biol. Med., 198: 500-512 (1991); Ying, Endocrine Rev. 9:262 Recombinant activin was also found to stimulate pituitary LH and FSH in the adult male macague. McLachlan et al., Endocrinol., 125: 2787-2789 (1989). Activin and inhibin regulate the growth and functions of a variety of cell types. They may be involved in diverse biological processes including erythropoiesis, bone formation, placental and gonadal steroidogenesis, neuronal survival, and embryologic mesodermal induction

Accordingly, there is provided a method for enhancing the fertility potential of oocytes comprising culturing the oocytes in vitro in a suitable serum or substrate comprised in a contained such as e.g. a petri dish or a test tube comprising a material according to the present invention, optionally a serum comprising an effective amount of inhibin, activin, or a combination of inhibin and activin, or a material coated with an effective amount of inhibin, activin, or a combination of inhibin and activin. The ovaries from which the oocytes are recovered are preferably unstimulated, but they may also be stimulated with, for example, elevated levels of endogenous or exogenous gonadotropins.

In a further aspect, the invention provides a method for increasing the rate of maturation of immature oocytes comprising culturing the oocytes in vitro, optionally with an effective amount of a combination of inhibin and activin; in a suitable serum or substrate comprised in a contained such as e.g. a petri dish or a test tube comprising a material according to the present invention.

In another aspect, the invention provides a method for fertilizing oocytes comprising removing oocytes from a follicle of an ovary, culturing the oocytes in a suitable serum or substrate comprised in a contained such as e.g. a petri dish or a test tube comprising a material according to the present invention, optionally with an effective amount of inhibin, activin, or a combination of inhibin and activin, and mixing the cultured oocytes with spermatozoa, resulting in fertilization.

In a still further aspect, the invention provides a method for storing and then enhancing the fertilization potential of oocytes comprising cryopreserving immature oocytes, thawing the cryopreserved oocytes, and culturing the thawed oocytes in vitro in a suitable serum or substrate comprised in a contained such as e.g. a petri dish or a test tube comprising a material according to the present invention.

While cryopreservation can take place by any means, in one aspect the cryopreservation procedure involves cooling oocytes immersed in a cryoprotective solution to a temperature of no more than about −60.degree. C., and storing the cooled oocytes at a temperature of no more than about −60.degree. C.

The ability to culture oocytes in vitro in a suitable serum or substrate comprised in a contained such as e.g. a petri dish or a test tube comprising a material according to the present invention so as to enhance their capability for fertilization and/or to enhance the rate and degree of maturation of immature oocytes could contribute substantially to a gamete pool if the culturing culminated in fertilization and normal embryonic development.

The enhancement of the quality of oocytes is expected to play a significant role in human-assisted reproductive technologies (ART) including IVF. Moreover, oocytes from diverse species that would otherwise be wasted can now be employed, such as immature oocytes obtained from primate species at necropsy, immature oocytes obtained during surgical intervention such as oophorohysterectomy, or immature oocytes recovered during hyperstimulation protocols or in natural cycles in the context of an IVF program. Also, oocytes can be removed from cancer patients, such as those with ovarian cancer, prior to chemotherapy, and wedge resection oocytes can be retrieved from gonadotropin-resistant women.

In addition, in vitro culturing of immature oocytes as practiced in accordance with this invention could minimize incubation times, improve the quality of incubated oocytes, and generally lead to knowledge that will improve IVF outcome. In this respect, increasing the quality of the oocyte without the use of stimulants such as clomiphene citrate and FSH/LH might limit multiple births if fertilization of one good-quality oocyte, rather than multiple irregular oocytes, can be achieved.

Additionally, this aspect of the present invention, when combined with cryopreservation of immature or mature oocytes and fertilization in the context of an ART cycle could circumvent ethical problems associated with the banking of human embryos in that one avoids the freezing of a living being. Also, maturation in vitro also has a time advantage in that one day rather than the typical 48-72 hours is required for maturation in vitro.

As used herein, the term “oocytes” refers to the gamete from the follicle of a female animal, whether vertebrate or invertebrate. Preferably, the animal is an endangered species and/or a mammal, and more preferably is a sports, zoo, or other animal whose oocytes would be desirable to save due to superior breeding, such as race horses, an endangered mammalian species, a non-human primate, or a human. “Endangered species' for purposes herein refers to a species of animal that has been deemed to be endangered by the U.S. Endangered Species Act of 1973 or its global counterpart, the World Conservation Union. Typically, the population of an endangered species is threatened due to overhunting, disease, and/or natural habitat destruction so that it can no longer survive in adequate numbers to maintain the species. Examples of endangered species include northern spotted owls, panda bears, highland gorillas, orangutans, chimpanzees, Siberian tigers, elephants, black-crested macagues, golden lion tamarins, etc.

“Immature” oocytes refers to oocytes that are viable but incapable of fertilization without additional growth or maturation. Oocytes recovered from unstimulated” follicles or ovaries are natural oocytes obtained from follicles or ovaries that were not treated with any gonadotropins or other hormones or agents to stimulate maturation of the oocytes. Oocytes recovered from “stimulated” ovaries may be either mature or immature. Subjective criteria to estimate the viability and maturity of the ovum that can be done microscopically after removal of the ovum from the follicle include assessing the number and density of surrounding granulosa cells, the presence or absence of the germinal vesicle, and the presence or absence of the first polar body.

Oocytes from unstimulated ovaries generally have two or more layers of surrounding condensed granulosa cells, a germinal vesicle, and no polar body, whereas oocytes from stimulated ovaries generally have an expanded granulosa cell layer called the cumulus, no germinal vesicle, and one polar body. Maturity may be measured by the number and density of surrounding granulosa cells, the presence or absence of the first polar body, and the thickness of the zona pellucida, as well as by oocyte resumption of meiotic maturation as expressed by the percentage of GV intact oocytes that undergo GVBD and/or that reach MII after 48 hours of culturing. See also Sathananthan et al., in Ultrastructure of the Ovary, supra, for ways to assess nuclear and cytoplasmic maturation of mammalian oocytes.

As used herein, the expression “enhancing the fertilization potential of oocytes” refers to increasing the quality of the oocyte so that it will be more capable of being fertilized and producing a viable embryo than would otherwise be the case, and also refers to increasing the extent (degree or percentage) of maturation of immature oocytes. Maturation is assessed as described above and quality can be assessed by appearance of the oocytes from photographs and by the IVF rate. Criteria to judge quality of the oocyte by visual means include, for example, their shape, cumulus expansion, GVBD, and extrusion of the first polar body. Immature GV oocytes usually have a compact cumulus and a tight layer of corona: cells, while maturing MI oocytes have an expanding cumulus and matured MII oocytes have an expanded cumulus. Also, GV oocytes usually have an eccentric nucleus and no polar body. Maturing oocytes at MI have no nucleus or polar body but do have a spindle. The mature oocytes have a single polar body in the perivitelline space and an MIT spindle. In addition, immature or atretic oocytes have a more compact and smooth zona, while mature MII oocytes have a spongy, meshlike appearance. Fertilized ova completing meiosis have two polar bodies in the perivitelline space and two pronuclei in the ooplasm. This latter stage can be measured by using Normarski inverted microscopy or phase microscopy after the cumulus cells are removed by gentle pipetting or dissection.

As used herein, the expression “increasing the maturation rate of immature oocytes” refers to increasing the rate at which maturation of the oocytes occurs over time, whether at the GVBD stage, at the MII stage, or both.

“Spermatozoa” refers to male gametes that can be utilized to fertilize the oocytes herein.

As used herein, the term “inhibin” refers to the heterodimers of alpha and beta chains, of inhibin, prepro forms, and pro forms, together with glycosylation and/or amino acid sequence variants thereof.

As used here, the term “activin” refers to homo- or heterodimers of beta chains of inhibin, prepro forms, and pro forms, together with glycosylation and/or amino acid sequence variants thereof.

Preferably, the inhibin and activin useful herein are human inhibin A or B and human activin A, AB, or B, most preferably human inhibin A and human activin A or human activin B.

The present invention in one preferred aspect relates to enhancing the fertility potential of animal oocytes, especially those of mammals, including sports, zoo, pet, and farm animals-such as dogs, cats, cattle, pigs, horses including race horses, monkeys, and sheep, endangered species, and humans.

The methods of this aspect of the invention involve first removing the oocytes, preferably immature oocytes, from follicles in the ovary. This is suitably accomplished by conventional techniques, for example, using the natural cycle as described below, using anovulatory methods, during surgical intervention such as oophorohysterectomy, during hyperstimulation protocols in the context of an IVF program, or by necropsy.

In the natural cycle, when the schedule of ovarian events progresses as expected, a burgeoning follicle(s) on the ovarian surface can be viewed near midcycle by ultrasound or laparoscopy, having distended vessels and substantial translucence. This is the familiar appearance of the dominant follicle near ovulation. A needle is passed into the follicle and its contents, which may be a single oocyte, are aspirated. Oocyte removal and recovery is suitably performed by means of transvaginal ultrasonically guided follicular aspiration. Following evacuation, the follicle collapses. After the follicle is aspirated, the ovum is recovered and examined microscopically to assess its condition. Additional smaller follicles may be aspirated in turn. Subjective criteria to estimate the normality of the ovum include assessing its maturity by the-number and density of surrounding granulosa cells, the presence or absence of the first polar body, and the thickness of the zona pellucida, as well as other criteria mentioned above.

However, as stated above, the invention is not limited to use of immature oocytes. Thus, suitable oocytes include those that are from ovaries stimulated by administration to the oocyte donor of a fertility agent or fertility agent enhancer, so that the oocytes are in a greater state of maturity than oocytes from unstimulated ovaries. Examples of agents used to induce such controlled multiple follicular maturation include inhibin administered directly to the ovary (WO 91/10445, supra), clomiphene citrate or human menopausal gonadotropins, e.g., FSH as described in U.S. Pat. No. 4,845,077, or a mixture of FSH and LH, and/or human chorionic gonadotropins.

A gonadotropin releasing hormone antagonist may be administered to decrease the marked individual variability in response to human menopausal gonadotropin therapy. Typical gonadotropin hormone releasing antagonists are described by Rees et al., J. Med. Chem., 17: 1016 (1974); Coy et al., Peptides, 1976 (Loffed Ed., Editions de L'Universite de Bruxelle 1977) p.463, Beattie et al., J. Med. Chem., 18: 1247 (1975); Channabasavaiah et al., Biochem. Biophys. Res. Commun., 86: 1266 (1979); and U.S. Pat. Nos. 4,317,815 and 4,431,635. These include (Ac-pCIPhe.sup.1, pCIPhe.sup.2, DTrp.sup.3, DArg.sup.6, DAla.sup.10)GnRH HCl, >D-Phe.sup.2 !-LHRH, >DPhe.sup.2, D-Phe.sup.6 !-LHRH, >D-Phe.sup.2, Phe.sup.3, D-Phe.sup.6 !-LHRH, >DPhe.sup.2, D-Trp.sup.3, D-Phe.sup.6 !-LHRH, >D-p-F-Phe-D-Ala.sup.6 !-LHRH, and >Ac-D-Phe.sup.1, D-Phe.sup.2, D-Trp.sup.3,6 !-LHRH.

These fertility agents are used in the amounts typically employed for such agents. For example, if FSH is used, preferably the effective amount given to the female before the oocytes are collected is a daily amount of about 70 to 220 I.U./kg, more preferably 1.5 to 4.0 I.U./kg. If a gonadotropin releasing hormone antagonist is used in conjunction with FSH, preferably the daily amount of gonadotropin releasing hormone antagonist is about 1.0 to 4.0 mg/kg, more preferably 1.5 to 2.5 mg/kg. Further details on administration of these latter agents can be found in U.S. Pat. No. 4,845,077.

Once the desired oocytes have been isolated (e.g., viable oocytes selected from microscopic examination), they are suitably cultured in a accordance with this invention in a suitable serum or substrate comprised in a contained such as e.g. a petri dish or a test tube comprising a material according to the present invention comprising a substratum, preferably, but not limited to polystyrene, or cryopreserved for storage in a gamete or cell bank for future culturing. If they are not to be frozen first, the oocytes should be cultured no more than about 48 hours after aspiration from the follicle or until the first polar body is released. If they are frozen, when it is desired to use them, they are thawed and then cultured by the invention method described herein.

Development of a cryoprotective methodology requires optimization of each individual component in the process through independent study followed by an integrated approach, combining optimal components, to identify the final process. Optimal freezing, storing, thawing, and rinsing procedures that are compatible with maintaining maximal viability are identified. Any method for freezing the oocytes can be utilized. For example, an ultrarapid freezing technique can be employed, as described in Trounson et al., Fertil. Steril., 48: 843-850 (1987) and Vasthevan et al., Fertil. Steril., 58: 1250-1253 (1992). Specific protocols for cryopreserving epithelial sheets and blood vessels that may be useful in the present invention are described in U.S. Pat. Nos. 5,145,770 and 5,145,769, respectively, the disclosures of which are incorporated herein by reference. One detailed method for cryopreservation of oocytes is set forth below, where modifications can be made as necessary to suit the individual treatment.

First, the oocytes are equilibrated in a cryopreservative solution for a time sufficient to allow the cryopreservative to mix thoroughly with and/or displace the water within and between the oocytes. Second, the oocytes are cooled to at least about −60.degree. C., preferably to about −180.degree. C. to −196.degree. C., at a rate slow enough for the cryoprotected cells to avoid intracellular ice crystal formation and subsequent damage. The frozen oocytes may be stored for long periods at about −180.degree. C. or for shorter periods at higher temperatures, e.g., as high as about −60.degree.−65.degree. C. Third, before use, the oocytes are warmed at room temperature in air or other gas, and then thawed completely by rapid warming in, for example, a water bath. Fourth, the cryoprotectant is removed from the oocytes by rinsing in an isotonic buffer such as lactated Ringer's solution, or in the culture medium to be used for enhancing the fertilization potential of the oocytes.

Standard cryoprotective medium is composed of a physiologically balanced salt solution (e.g., cell culture medium) supplemented with bovine serum and a cryoprotectant such as glycerol, propanediol, or dimethylsulfoxide, cell-penetrating, glass-forming agents. These cryoprotectants have been used successfully for cryopreserving cells in suspension, including fertilized embryos. In addition, non-cell-penetrating, glass-forming agents may be added as described in U.S. Pat. No. 5,145,770, supta, as well as the cryopreservative mentioned in U.S. Pat. No. 5,145,769, supra.

The cryopreservation process in general requires immersing the oocytes to be frozen in cryoprotective medium for a time sufficient to permit equilibration of the cells with cryoprotectant. Generally, the equilibration time is for up to about two hours or more in cryoprotectant prior to freezing without affecting the viability of the cells. The equilibration is conducted more typically for about 20-30 minutes, at about 17.degree. C. to about 30.degree. C. typically at room temperature, in a cryoprotective solution, in a shallow storage dish.

Following eguilibration, the oocytes and the cryoprotectant solution are transferred to a straw or vial that is sealed so that it is gas-and water-tight. The oocytes in the sealed container are cooled to at least about −60.degree. C. (e.g., with dry ice), preferably below −120.degree. C., and to promote longer-term storage, to approximately −180.degree. C. to about −196.degree. C. The cooling rate preferably is slow (e.g., no more than about 1.degree. C./min.) from about 0.degree. C. to at least −30.degree; C. This serves to discourage ice crystal formation. Preferably, cooling is conducted at the outset in a rate-controlled cooling device such as a commercial programmable cell freezer (Cryomed, Inc. No.; 1010/2700) to a temperature of about −30.degree. C. to −100.degree. C., preferably about −80.degree. C. to −85.degree. C., and then the contents are transferred to a liquid nitrogen storage vessel and maintained ill vapors of liquid nitrogen to reduce its temperature further.

The preferred freezing protocol cools the oocytes in the sealed container until the oocytes are approximately 4.degree. C. Then the oocytes are cooled at about 1.degree. C. per minute to about −6.degree. or −7. degree. C. and the solution is seeded. After an equilibration period of about 10 minutes, the mixture is cooled at about 0.3.degree. C. per minute. Once the temperature of the oocytes reaches at least about −30.degree. C., and preferably at least about −85.degree. C., the container is transferred toga liquid nitrogen refrigerator and stored at about −180.degree. C. (nitrogen vapors) or about −196.degree. C. (liquid nitrogen),

Thawing the oocytes is suitably accomplished by removing the sealed container from the liquid nitrogen refrigerator and preferably keeping it at room temperature in air for about 1 minute and up to about 3 to 5 minutes. This produces a warming rate of between about 20.degree. C./min. and about 100.degree. C./min. The oocytes may then be heated to room temperature without regard to the rate of heating. Preferably the last stage is conducted by submerging the sealed container in a water bath until the oocytes are thawed. This prevents the zonae pellucidae surrounding frozen oocytes from cracking. Alternatively, the water bath is eliminated and the oocytes are thawed at room temperature; however, this takes longer than the water bath and often has the effect of reducing cell viability.

Once the oocytes are thawed, the container is suitably opened and the cryopreservative solution replaced by an isotonic buffer solution at physiological pH (about 6.8 to 7.4), preferably FAD medium or lactated Ringer's solution or the culture medium to be used to enhance the fertilization potential of the oocytes, to dilute out the cryoprotectant. Not all isotonic buffered solutions at physiological pH may be acceptable for dilution of cryoprotectant; Phosphate buffered saline and standard saline may reduce viability significantly. The thawed oocytes are equilibrated preferably at about room temperature in rinsing buffer or culture medium preferably for about 15 minutes and may remain there for up to about 4 hours. Direct microscopic visualization can be used to determine if the oocytes are still viable as compared to non-frozen, non-stored control oocytes.

After placement in the rinsing solution for a sufficient period of time, the oocytes can then be cultured in a suitable serum or substrate comprised in a contained such as e.g. a petri dish or a test tube comprising a material according to the present invention comprising a substratum according the invention, preferably, but not limited to polystyrene.

Alternatively, after removal from the follicle the oocytes are cultured in a suitable serum or substrate comprised in a contained such as e.g. a petri dish or a test tube comprising a material according to the present invention comprising a substratum, preferably, but not limited to polystyrene, and then frozen before fertilization is carried out; as described below. The culturing optionally takes place in a suitable culture medium that includes at least inhibin, activin, or a combination of inhibin and activin in an amount effective to enhance the fertility potential of oocytes in general, and to enhance the rate and the extent of maturation of immature oocytes and the quality of the oocytes in particular. However, the culture medium may be any state of the art culture medium including a culture medium generally containing physiologically balanced salts, energy sources, and optionally also antibiotics, i.e. a medium that is suitable for the species whose oocytes are being treated Examples of suitable media for certain species such as humans and monkeys include human tubal fluid (HTF) as obtained from Quinn et al., Fertil. Steril., 44:493 (1985), supplemented with 10% heat-inactivated maternal or fetal cord serum, which is typically used for IVF and embryo culture, TALP, as obtained from Boatman, in In Vitro Growth of Non-Human Primate Pre- and Peri-implantation Embryos, ed. Davister, pp. 273-308 (New York: Plenum Press, 1987), Ham's° F-10 mediun, Menezo's B.sub.2 medium (BioMerieux SA, France), Earles medium (Sigma Chemical Co., St Louis, Mo.), etc. General reviews describing these types of media include Menezo and Khatchadourian, “The Laboratory Culture Media,” Assisted Reproduction Reviews, 1: 136 (1991) and Lease, “Metabolism of the Preimplantation Mammalian Embryo,” Oxford Reviews of Reproductive Biology, 13: 35-72 (1991), ed. S. R. Milligan, Oxford University Press. The practitioner will be able to devise the necessary medium suitable for the species. The pH of the culture medium is generally about 7 to 8, more preferably about 7.2-7.6.

The conditions required for culturing the oocytes depend on, for example, the type and number of oocytes being treated. Typically the culturing temperature is in the range of about 36.degree.−39.degree. C., although temperatures outside this range may also be suitable, for example, about 35.degree.-40.degree. C. The culturing time is at least about 1 hour, preferably about 4 to 100 hours, and more preferably about 12 to 36 hours. Typically the culturing environment contains about 95-100% humidity, 5% CO.sub.2, 5% O.sub.2, and 90% N.sub.2. Vessels of tissue-culture-grade plastic useful for carrying out the culturing include test tubes, vials, organ culture dishes, petri dishes, or microtiter test plates.

Once the oocytes are matured or stimulated to the point of being capable of fertilization, as indicated by any one or more of the factors noted above or others, they are mixed with suitable spermatozoa from the same species, resulting in fertilization. The fertilization with sperm can be carried out in vitro by known techniques including sperm injection or in vivo, including those indicated below and newer technologies for effecting fertilization.

Examples of human in vitro fertilization and embryo transfer procedures that may be successfully carried out using the method of this invention include, e.g., in vitro fertilization and embryo transfer, (IVF-ET) (Quigly et al., Fertil. Steril. 38: 678>1982!), gamete intrafallopian transfer (GIFT) 4 Molloy et al., Fertil. Steril, 47: 289>1987!), and pronuclear stage tubal transfer (PROST). Yovich et al., Fertil. Steril, 45: 851 (1987). Successful such procedures are positively correlated with the number of oocytes retrieved and the number of viable embryos transferred

In IVF-ET, the oocytes are inseminated with washed and migrated spermatozoa (typically 100,000 to 200,000 per oocyte). Fertilization is assessed typically 12 to 18 hours after insemination and the oocytes are transferred to growth media such as HTF, Ham's F-10, or Earles. Only normal embryos are transferred to the patients at the 2- to 8-cell stage at typically 48 to 56 hours after retrieval.

General protocols for IVF include those disclosed by Trounson et al., supra; Trounson and Leeton, in Edwards and Purdy, eds., Human Conception in Vitro (New York: Academic Press, 1982), and Trounson, in Crosignani and Rubin, eds., In Vitro Fertilization and Embryo Transfer, p. 315 (New York Academic Press, 1983), the disclosures of all of which are incorporated herein by reference.

The threat of non-hormone-induced luteal phase hormonal deficiency that may occur in IVF may be ameliorated by administration of progesterone.

For the PROST protocol, all procedures for oocyte aspiration, enhancement of fertilization potential, semen suspension preparation, and insemination are performed using the same procedure as in the IVF program. After the assessment of oocyte fertilization, pronuclear oocytes are transferred into the fallopian tube by the same procedure as in the GIFT program, wherein the fallopian tube is catheterized as described by Molloy et al., Pert. Steril., 47: 289 (1987).

Another method and apparatus for IVF is found in WO 92/20359 published 26 Nov. 1992, wherein the oocytes to be fertilized are placed in individual, low-volume oocyte chambers disposed about the periphery of a microdrop of fertilization medium A sperm sample, particularly an unfractionated sperm sample, is then placed in the center of the microdrop. Motile sperm tend to move rapidly toward the periphery of the microdrop, resulting in an in situ separation of motile from non-motile sperm. Once at the periphery, fertilization by sperm that enter the oocyte chambers is facile because of the low volume of the chamber.

Biocompatible Materials and Methods for Culturing Epidermal Cells

In another embodiment of the present invention there is provided the use of tissue culturing plasticware comprising a substratum including a pretreated substratum, preferably, but not limited to polystyrene, for growing vertebrate cells including human cells including human skin cells, said method comprising the steps of contacting a vertebrate cell including an epidermal cell with a biocompatible material according to the invention, and growing said cells under conditions suitable for such growth.

The vertebrate skin thus generated preferably comprises two principal layers, an outer epidermal layer and a dermal layer lying under the epidermal layer. In order for skin to retain its normal appearance and to function fully in a normal manner, both layers of the skin need to be present.

The present invention thus aides a number of skin grafting methods by providing an essential component of reconstructive surgery after burns, trauma, tumor excision, and correction of congenital anomalies. There are approximately 1 million burns per year in the U.S. alone, which result in about 100,000 admissions to burn units, about ⅓ of which require skin grafting. Skin grafting in reconstructive surgery is often required to alleviate deformity. The best possible skin available for grafting would be skin from the same patient taken from a donor site elsewhere on the body (referred to as an autograft).

Suitable skin graft donor sites, however, are limited not only by body surface area, but can also be affected by previous graft harvest or trauma. There are times, therefore, when donor skin is limited and the amount of skin required for grafting is quite large, so that sufficient autografts are not available. Because of the importance of the ski in preventing infection, either the donor skin must be used to cover a larger area than it originally covered or some suitable replacement material must be used.

Currently, several techniques are used to enhance the amount of donor area skin. Meshing of donor skin. (slitting the skin to form an expandable mesh pattern) is used to increase the total area of graft. However, meshing is only minimally able to increase graft size while it significantly detracts from the appearance of grafts, making them unacceptable for reconstruction on the face, and far from idealon the hands, arms, and neck. In patients suffering from large burns with limited donor skin sites, cadaver allografts are commonly used for temporary skin coverage, but ultimately such allografts are rejected, and a permanent autograft is required. In addition, allografts also pose a risk of infection of the recipient by virses or other disease-causing organisms present in the donor, such as infection by human immunodeficiency virus or hepatitis virus,

To aid in the grafting of patients with limited donor areas, cultured epithelial cells derived from the patient being treated have been utilized in many grafting applications. In general, the cells are used in the form of a monolayer of epithelial cells grown on a culture medium. Preparation of such cultures requires many weeks or months, and the product is quite difficult to handle because of its fragility, even when multiple epidermal cell layers are used to form a multi-layer skin substitute.

Harvesting of multiple skin grafts from the same donor site is often used, but such harvesting requires weeks to months between procedures for new skin to grow on the donor site. It is also a very traumatic technique, since multiple painful operations must be undertaken.

Tissue expansion techniques, which are in vivo techniques, have been used in plastic surgery for over a decade and can be helpful in increasing the area of donor tissue. By placing an expander subcutaneously and frequently injecting it with saline, skin can be expanded and its surface area increased. This allows reconstruction with local skin after expansion of an adjacent tissue bed. Expanders are not ideal, however, because they require multiple procedures. When local tissue is of poor quality, as might be the case in a patient who has undergone multiple reconstructions or irradiation or has been burned, expanders are not a viable option.

The present invention solves the problems associated with the prior art solutions by providing a technique that provides a large surface area of normal skin from a small donor skin segment. The solution embodies the use of a biocompatible material according to the invention for culturing a monolayer of epithelial cells grown on a culture medium.

The biocompatible material in one embodiment is a porous membrane material which can be either bioerodable (e.g. polylactide), or non-erodable (e.g. polycarbonate) as described in detail herein elsewhere. Such materials are preferably used as culture substratum for epidermis keratinocytes) and cutis (fibroblasts). The present invention is capable of improving the biocompatibility of the membranes without changing parameters such as e.g. oxygen permebaility required for stratification. The invention provides a better growth substratum for the cells in comparison to commercial PC membranes as well as other substarta.

The skin is preferably an autograft, and the skin is preferably available for medical and surgical purposes in a substantially shorter period of time than presently possible. Accordingly, the present invention is of great benefit to reconstructive surgery patients because it limits the number of surgical procedures required on a patient. The invention also aims to increase survival by closing wounds more promptly in a patient who requires a large amount of skin grafting.

Surface coating of Haemodialysis Membranes to Reduce Adsorption and Activation of Blood Components

Haemodialysis is the essential therapy to save the life of patients with acute and chronic kidney failure. Extracorporal oxygenation techniques use membranes to help patients with a cardiopulmonary bypass during open heart surgery, or patients in intensive care units, achieve enrichment of whole blood with oxygen.

A major problem associated with state of the art haemodialysis methods is a pronounced lack of “biocompatibility” of blood contacting devices including haemodialysis membranes.

Tolerance of hemodialysis in patients is affected by various factors such as the physical and mental state of the patient, the sterile environment, and especially the dialyzer, with the biocompatibility of the hollow fiber in the dialysis module being an important factor. In addition, the surface properties of the polymer, the membrane structure, and the dialyzer design have a significant influence on biocompatibility in dialysis treatment.

The chemically different structures of the various polymers play an important role in biocompatibility, as for example in complement activation (C5aformation), hemolysis, and thrombogenesis.

In addition to the fact that dialysis membranes made of synthetic or natural polymers, when used in artificial kidneys, can very easily cause blood clotting which is largely prevented by suitable treatment with drugs, there is another effect that frequently occurs in dialysis membranes made of regenerated cellulose. Specificially when treating a kidney patient using a dialyzer with cellulose membranes a transient decrease in the number of leucocytes can occur at the beginning of dialysis treatment. This effect is known as leucopenia and must be at least largely suppressed or prevented by modifying the membrane.

Leucopenia in dialysis is most strongly evident 15 to 20 minutes after the star, when the neutrophils (in other words, the leucocytes that can be stained with neutral dyes or simultaneously with acid and basic dyes) can disappear almost completely. Then the number of leucocytes recovers within about 1 hour, back to nearly the initial value or even above the latter. If a new dialyzer of the same kind is connected after the leucocytes recover, leucopenia again occurs to the same degree.

Cellulose membranes cause pronounced leucopenia. Although the clinical significance of leucopenia has not yet been scientifically explained, it is desirable to have a dialysis membrane for hemodialysis that does not show the effect of leucopenia but does not adversely affect the other highly desirable properties of dialysis membranes made of regenerated cellulose.

In hemodialysis using membranes made of regenerated cellulose, pronounced complement activation has been observed along with the leucopenia. The complement system within the blood serum is a complex plasma enzyme system composed of many components which works in different ways to defend against injury by invading foreign cells (bacteria, etc.). If antibodies against the invading organism are available, activation is possible in a complement-specific manner by the complex of antibodies with antigen structures of the foreign cells, otherwise complement activation takes place along an alternative path through special surface features of the foreign cells.

The complement system is based on a number of plasma proteins. After activation, these proteins react specifically with one another in a certain sequence and finally a cell-damaging complex is formed which destroys the foreign cell.

Peptides are released front individual components, triggering inflammation phenomena and possibly also having undesired pathological consequences for the organism. It is assumed that activation in hemodialysis membranes made of regenerated cellulose takes place via the alternative path. These complement activations have been determined objectively by detection of complement fragments C3a and C5a. Reference is made in this connection to D. E. Chenoweth et al., Kidney International, Volume 24, Pages 764 et seq., 1983 and D. E. Chenoweth, Asaio-Journal, Volume 7, Pages 44 et seq., 1984. It is generally desirable to reduce or eliminate complement activation as much as possible in hemodialysis.

Synthetic membranes developed for haemodialysis applications are moderately wettable to hydrophobic. One of many problems associated with these membranes is the strong adsorption of proteins to the membranes. This adsorption not only deteriorates the transport properties due to so-called fouling, but also leads to activation of defence systems in blood, such as the complement system or the coagulation system. The activation is caused by the adsorption of specific complement and coagulation factors to the membranes.

Another problem is the adsorption of adhesive proteins, such as fibrinogen, von Will-brand factor, Fibronectin and vitronectin, and subsequent conformational changes that exposes novel epitopes leading to the attachment and activation of blood cells including platelets, neutrophils and monocytes. Serious consequences of these events are often diagnosed as the local formation of thrombi and emboli that may be fatal for the patient. Adhesion and activation of the white blood cells is also connected with inflammation with local or systemical signs.

Anticoagulation treatments with heparin or coumarin derivatives represent one inadequate attempt to reduce the negative side effects of haemodialysis membranes on the coagulation system. In fact, there may be conditions when a high degree of anticoagulation results in excessive internal bleeding and threatens the life of the patient.

To overcome these difficulties attempts have been made to immobilise anticoagulant agents, like heparin, or to make surfaces on dialysis membranes inert by covalent coupling of PEO or similar substances. However, this has created the problem that the dialysis membranes have a reduced permeability and are no longer useful for the intended application.

The present invention provides a solution to the observed problems by reducing the quantities of adsorbed proteins and—at the same time—stabilising the natural conformation of the adsorbed proteins. The membrane surface modified according to the present invention would not be recognised as a foreign object, and this eliminates the activation of the different defence systems in blood. A further advantage of the present invention is that the degree of modification resulting from macromolecule including PEG attachment does not reduce the permeability of the membranes to any signification extent. Hence, they would still be suitable for the application as haemodialysis membrane. Accordingly, surface coating of haemodialysis membranes aimed at reducing adsorption and activation of blood components represents a focus area of the present invention.

The present invention in one aspect relates to a dialysis membrane comprising a porous material according to the invention comprising a substratum according to the invention, said membrane being characterized by the fact that its properties can be adapted to as many dialysis parameters as possible and that it is economical to manufacture and process. This goal is achieved by dialysis methods and membranes according to the present invention comprising a porous material comprising a substratum according to the present invention.

In one aspect the present invention pertains to a dialysis apparatus comprising:
i) at least one dialyzer with a membrane, preferably a membrane comprising a material according to the invention comprising a substratum according to the invention, said membrane dividing said dialyzer into a first chamber and a second chamber,
ii) wherein said first chamber being in a first circuit connected with a single lumen catheter means and a storage means and comprising a means for supplying a dialysis fluid, and
iii) wherein said second chamber being connected via a second circuit with a means for preparing said dialysis fluid,
iv) wherein said second circuit comprising pump means and a dialysis filter divided by a membrane, preferably a membrane comprising a material according to the invention comprising a substratum according to the invention into a first chamber and a second chamber,
v) wherein said first chamber of said dialysis filter being in said first circuit, and
vi) wherein said second chamber of said dialysis filter being connected with said catheter means.

In preferred embodiments the dialyzer is preferably a haemodialyzer composing a cut-off limit of about 5,000-10,000 Dalton (molecular weight), and the dialysis filter is preferably a haemofilter with a cut-off limit of 20,000-40,0000 Dalton.

The first circuit preferably comprises a substance whose molecular weight is above that of the cut-off limit of the dialyzer and of the dialysis filter. The second chamber of the dialysis filter is preferably connected via an exit duct with the catheter means, said exit duct comprises a first sensor, the first chamber of the dialysis filter is connected via an outlet duct with the first chamber of the dialyzer, said outlet duct comprises a second sensor, and said first and second sensors are connected with a controlling means for controlling said substance.

The second chamber of the dialyzer preferably comprises an outlet duct comprising a third sensor connected with said controlling means, and the first circuit preferably comprises a substance whose molecular weight is above that of the cut-off limit of the dialyzer, but smaller than the cut-off limit of the dialysis filter.

In one embodiment the exit duct comprises a means for controlling the ultrafiltration by comparing the starting concentration of the substance and the concentration of the substance during the treatment of the patient.

In another aspect of the present invention there is provided a dialysis apparatus comprising:
i) at least one dialyzer with a membrane dividing a space therein into first and second chambers, said membrane preferably comprising a material according to the invention comprising a substratum according to the invention,
ii) a single lumen catheter,
iii) a first circuit joining said first chamber with; said catheter,
iv) a second circuit,
v) means for preparing dialysis liquid and joined with said second chamber via said second circuit, wherein said means for preparing dialysis liquid is preferably in the form of a constant-volume, balanced system.
vi) at least one pump in each of said first and second circuits,
vii) supply and outlet ducts,
viii) a dialysis filter, preferably a filter comprising a material according to the invention comprising a substratum according to the invention, said filter being joined with said supply and outlet ducts and having first and second chambers therein and so placed in said first circuit that with said supply duct and said outlet duct and the first chamber of the dialyzer the first chamber of the dialysis filter forms a closed circuit,
ix) a connection duct joining said second chamber of said dialysis filter with said catheter,
x) a first pump placed in said supply duct, preferably a peristaltic pump,
xi) a second pump placed in said outlet duct, preferably a peristaltic pump,
xii) and a storage vessel joined with same at a point downstream from said second pump.

The apparatus preferably further comprises i) means for withdrawing ultrafiltrate from the means for preparing dialysis liquid and/or ii) means for alternating operation of the first and second pumps, said pumps shutting off said supply and outlet ducts when said pumps are not in operation and/or iii) a drip chamber connected in said supply duct downstream from the dialyzer, such drip chamber having a means for clearing air and a liquid level sensor and/or iv) a monitoring system including a monitoring unit and at least one sensor fitted to at least one of ducts selected from the group consisting of; a duct joined with said dialysis filter, said outlet duct, said duct joining said dialyzer with said dialysis filter, wherein said sensor is preferably electrically joined with an ultrafiltration controller for controlling the ultrafiltration pump on the basis of a comparison with the initial concentration of the substance for control of the ultrafiltration.

The dialysis filter is preferably placed in a circuit loop in which there is a substance whose molecular weight is above that of the cut-off limit of the dialyzer and of the dialysis filter, preferably a substance in said circuit joining said dialyzer with said filter, whose molecular weight is greater than the cut-off limit of the dialyzer but smaller than the cut-off limit of the dialysis filter.

The apparatus may further comprise a vessel for additional liquid and a further duct joining same with said supply duct, said further duct having means for controlling the flow of liquid therethrough and being operated synchronously with said first pump, wherein said flow controlling means preferably includes a pump or a hose clamp.

In one embodiment the apparatus is comprising an ultrafiltration controller, a wire electrically joining the said flow controlling means with said controller, and an ultrafiltration pump joined with said controller, said controller causing said ultrafiltration pump to pump an amount of ultrafiltrate equal to an amount of liquid taken from said additional liquid vessel.

The apparatus may further comprise an exit duct connected with said dialysis filter, means detachably joining said connection duct with said exit duct in a resting condition, a controller electrically joined with said first and second pump for so controlling said pumps in a swilling and disinfection phase that the pumping rate of said first pump is greater than that of said second pump.

The apparatus may further comprise a sterilely hermetic, hydrophobic filters preferably a filter comprising a material according to the invention comprising a substratum according to the invention, preferably a substratum being selected from the group consisting of moderately to highly hydrophobic polymer substrata, such as, but not limited to, polysulfon and derivatives thereof, polyethersulfon and derivatives thereof, sulfonated polysulfon and derivaties thereof, polyacrylonitrile and derivatives thereof, polymethylmethacrylate and derivatives thereof, polycarbonate and derivatives thereof, and polyamide and derivatives thereof.

Besides a sterilely hermetic, hydrophobic filter, the apparatus may further comprise a valve, an intermediate duct joining said hydrophobic filter and said valve in series between said second pump and said dialysis filter, said valve being electrically joined with said controller, which is electrically connected with said ultrafiltration pump.

In a further embodiment the apparatus comprises a degassing vessel, controlling means for switching said apparatus into a checking phase in which said controller opens said valve and turns on said ultrafiltration pump, a pressure sensor fitted to said exit duct and a liquid level sensor placed on said degassing vessel for ascertaining a time pressure relation.

The means for preparing dialysis liquid is preferably designed to be switched, while in a filling phase, into an open mode of operation, the valve is open to let off air, the first pump is kept turned on till liquid emerges at said hydrophobic filter and subsequently said second pump is put into operation with a pumping rate below that of said first pump.

The controller preferably operates the means for preparing dialysis liquid so that after connection of the connection duct with the catheter of the patient a certain amount of liquid is discharged through said connection duct and the catheter.

The means for preparing dialysis liquid preferably includes a balanced chamber, a feed pump and means for operation of same in a cycle in step with operation of said first and second pumps so that spent dialysis liquid is displaced and filling takes place in a first cycle stroke and in a second stroke the content thereof is pumped through the said dialyzer using said feed pump, said first pump being operated in said second stroke and said second pump being operated in said first stroke.

Additional aspects of the present invention pertains to i) catheters, including diagnostic catheters, catheter components and tubing comprising at least a surface area comprising or essentially consisting of a material according to the invention comprising a substratum according to the invention, ii) drainage devices comprising at least a surface area comprising or essentially consisting of a material according to the invention comprising a substratum according to the invention, iii) blood filters comprising at least a surface area comprising or essentially consisting of a material according to the invention comprising a substratum according to the invention, iv) assay trays comprising at least a surface area comprising or essentially consisting of a material according to the invention comprising a substratum according to the invention, v) petri dishes comprising at least a surface area comprising or essentially consisting of a material according to the invention comprising a substratum according to the invention, vi) and culture flasks comprising at least a surface area comprising or essentially consisting of a material according to the invention comprising a substratum according to the invention.

Surface Coating of Blood-Contacting Devices Aimed at Improving Attachment and Growth of Endothelial Cells and Reducing Attachment and Activation of Blood Components Including Platelets

Large diameter artificial blood vessels made of TEFLON (PTFE) or DACRON (PET) as replacements for e.g. the aorta has been applied for many years with clinical success. However, there are presently no small diameter blood vessels available for replacement therapies. Even in the presence of anticoagulants rapid clotting and occlusion of small diameter blood vessels is observed. Large diameter vessels are also not free of the risk of clotting, and life-long anticoagulation therapies have to be performed in order to reduce the hazards of thrombosis for the patients.

The development of the idea of prosthetic vascular grafts has been a major goal of vascular surgery since the first grafts were used over 30 years ago. Most approaches have concentrated on creating a surface that is thromboresistant, with the majority of these efforts directed toward an improved polymer surface. Perhaps the ideal blood-surface interface is the naturally occurring human endothelium. If present on a prosthetic graft, it would offer many of the advantages of a native vessel. Unfortunately, endothelialization occurs only to a limited degree in prosthetic grafts when placed into humans.

Seeding endothelial cells onto preclotted prosthetic grafts prior to implantation has improved the endothelial cell coverage of grafts in animals, but this technique has had limited use in humans. See “Human Adult Endothelial Cell Growth in Culture”, Bruce Jarrell, et al., Journal of Vascular Surgery 1984, I(6), 757-764; Herring et al., “A Single and Staged Technique for Seeding Vascular Grafts with Autogenous Endothelium”, Surgery 1978, 84, 498-504; Graham et al., “Cultured Autogenous Endothelial Cell Seeding of Vascular Prosthetic Grafts”, Surg Forum 1979, 30, 204-6; Graham et al., “Expanded Polytetrafluoroethylene Vascular Prostheses Seeded with Enzymatically Derived and Cultured Canine Endothelial Cells”, Surgery 1982, 91, 550-9 and Dilley et al., “Endothelial Seeding of Vascular Prostheses”, Biology of Endothelial Cells, pp 401-11, Jaffe ed., The Hague: Martinus Nijhoff, 1984.

Over the past three decades, artificial grafts have been used to provide immediate restoration of blood flow to areas of ischemia as a result of atherosclerotic vascular disease. In addition, they have been used to provide vascular access for hemodialysis in patients with chronic renal failure, and in the repair of arterial aneurysms. Although initially successful at restoring perfusion to ischemic tissues, the long-term prognosis for these grafts is not encouraging. Over an extended period, grafts less than 4 mm in diameter lose their patency as they become occluded via fibrin deposition and cellular adhesion. This process appears to be secondary, and to be due in part to the thrombogenic nature of the nude (i.e., nonendothelialized) surface of the implanted prostheses. See Berger et al., “Healing of Arterial Prostheses in Man: It's Incompleteness”, Ann. Surg. 1972, 175, 118-27.

Many attempts have been made to improve the blood response of materials by reducing the thrombogenic potential of the materials by covering them with substances, such as PEO, aimed at making them inert, or covering them with a lining of endothelial cells. The latter approach is physiologically more relevant as endothelial cells normally have an anticoagulant activities. However, spontaneous coverage of the artificial blood vessel with endothelial cells is never observed in humans.

Thus, much current research is being aimed at either: (1) developing grafts with an artificial, non-thrombogenic surface, or (2) lining vascular prostheses with human endothelial cells, in the hope of producing a non-thrombogenic endothelial cell surface such as exists in native human vessels.

Endothelial cells from animal sources have been studied in culture since the 1920's. In 1973, Jaffe et al. successfully cultured endothelial cells from human umbilical veins and these cells have been characterized functionally. See Jaffe et al., “Synthesis of Antihemophilia Factor Antigen by Cultured Human Endothelial Cells”, J. Clin. Invest. 1973, 55, 2757-64; Lewis, “Endothelium in Tissue Culture”, Am. J. Anat. 1922, 30, 39-59; Jaffe et al., “Culture of Human Endothelial Cells Derived From Umbilical Veins”, J. Clin. Invest. 1973, 52, 2745-56. These cell cultures demonstrate a growth potential, but the total number of cells produced from a single umbilical vein is usually quite limited, in the range of a 10-100-fold increase in harvested endothelial cells.

While several techniques have been proposed to increase the number of cells produced in the use of human umbilical vein endothelial cells, the ability to culture endothelial cells in large numbers remains less than ideal. Some investigators have had some success in culturing human adult endothelial cells from pulmonary arteries and veins, but only for short periods of time. It has also been shown that human iliac artery endothelial cells may be cultured for a short number of passages. In a study by Glassberg et al., for example, it is reported that 50 to 500 viable cells can be obtained per 5-inch vessel segment, a very low yield. “Cultured Endothelial Cells Derived From Human Iliac Arteries”, In Vitro 1982, 18, 859-66. Fry et al. have reported successfully culturing human adult endothelial cells from abdominal arteries removed at the time of cadaver donor nephrectomy, but these cells also demonstrated early senescence.

It is apparent from existing techniques that it is difficult to produce enough cells to preendothelialize a graft with a reasonable amount of vessel from the donor patient. Rather than completely endothelializing a graft prior to implantation, the concept of subconfluent “seeding” of a preclotted graft developed. Seeding vascular grafts with autogenous endothelial cells has recently been shown to increase the rate of endothelial coverage of the grafts of experimental animals. See Herring et al. and Graham et al. supra. Once covered by endothelium, grafts in dogs have been shown to be less thrombogenic as measured by platelet reactivity, to be more resistant to inoculation from blood-born bacterial challenge, and to have prolonged patency of small-caliber vascular grafts. See Sharefkin et al., “Early Normalization of Platelet Survival by Endothelial Seeding of Dacron Arterial Prostheses in Dogs”, Surgery 1982, 92, 385-93; Stanley et al., “Enhanced Patency of Small Diameter Externally Supported Dacron Iliofemoral Grafts Seed with Endothelial Cells”, Surgery 1982, 92, 994-1005; and Watkins et al., “Adult Human Saphenous Vein Endothelial Cells: Assessment of Their Reproductive Capacity for Use in Endothelial Seeding of Vascular Prostheses”, J. Surg. Res. 1984, 36, 588-96.

A point of major concern when translating to human graft seeding has been the ability to produce enough endothelial cells with the use of human vascular tissue to allow seeding at a density high enough to attain endothelial coverage of the graft. Watkins et al., using human saphenous vein remnants following coronary artery bypass surgery were able to produce small quantities of endothelial cells in culture, and reported a low-fold increase in confluent cell area obtained in culture after 4 to 6 weeks.

Even if it were possible to substantially expand the number of endothelial cells available through vigorous culturing techniques, concerns would still remain concerning the “health” of these endothelial cells after as many as 40 or 50 population doublings. Furthermore, the incubation of such cells in cultures which are foreign to their natural environment raises further concerns about genetic alterations and/or patient contamination with viruses, toxins or other damaging materials.

Many endothelialization procedures are suggested in the literature. Investigations in this area have been complicated by the diverse nature of the endothelium itself and by the species to species differences which have been found relating to the behavior and characteristics of the endothelium. Fishman, “Endothelium: A Distributed Organ of Diverse Capabilities”, Annals of New York Academy of Sciences 1982; 1-8; Sauvage et al., “Interspecies Healing of Porous Arterial Prostheses”, Arch Surg. 1974, 109, 698-705; and Berger, “Healing of Arterial Prostheses in Man: Its Incompleteness”, supra Nonetheless, the literature is replete with reports of experiments involving the seeding of endothelial cells on various grafts, in various species, with a mixture of results. F. Hess et al., “The Endothelialization Process of a Fibrous Polyurethane Microvascular Prostheses After Implantation in the Abdominal Aorta of the Rat”, Journal of Cardiovascular Surgery 1983, 24(5), 516-524); W. K. Nicholas et al., “Increased Adherence of Vascular Endothelial Cells to Biomer Precoated with Extracellular Matrix”, Trans. Am. Soc. Artif Intern Organs 1981, 28, 208-212; C. L. Ives et al., “The Importance of Cell Origin and Substrate in the kinetics of Endothelial cell Alignment in Response to Steady Flow”, Trans. Am. Soc. Artif. Intern Organs 1983, 29, 269-274; L. M. Graham et al., “Expanded Polytetrafluoroethylene Vascular Prostheses Seeded with Enzymatically Derived and Cultured Canine Endothelial Cells”, Surgery 1982, 91 (5), 550-559; S. G. Eslin et al., “Behavior of Endothelial Cells cultured on Silastic and Dacron Velour Under Flow conditions” In Vitro: Implications for Prelining Vascular Grafts wit Cells, Artificial Organs 1983, 7 (1), 31-37; T. A. Belden et al., “Endothelial Cell Seeding of Small-Diameter Vascular Grafts”, Trans. Am. Soc. Artif Intern. Organs 1982, 28, 173-177; W. E. Burkel et al., “Fate of Knitted Dacron Velour Vascular Grafts Seeded with Enzymatically Derived Autologous Canine Endothelium”, Trans. Am. Soc. Artif Intern. Organs 1982, 28, 178-182; M. T. Watkins et al., “Adult Human Saphenous Vein Endothelial Cells: Assessment of Their Reproductive Capacity for Use in Endothelial Seeding of Vascular Prostheses”, Journal of Surgical Research 1984, 36, 588-596; M. B. Herring et al., “Seeding Arterial Prostheses with Vascular Endothelium”, Ann. Surg. 1979, 190(1), 84-90; A. Wesolow, “The Healing of Arterial Prostheses—The State of the Art”, Thorac. Cardiovasc. Surgeon 1982, 30, 196-208; T. Ishihara et al., “Occurrence and Significance of Endothelial Cells in Implanted planted Porcine Bioprosthetic Valves”, American Journal of Cardiology 1981, 48, 443-454; W. E. Burkel et al., “Fate of Knitted Dacron Velour Vascular Grafts Seeded with Enzymatically Derived Autologous Canine Endothelium”, Trans. Am. Soc. Artif Intern Organ 1982, 28, 178-182.

It has been previously recognized that human microvascular endothelial cells, that is, the cells which are derived from capillaries, arterioles, and venules, will function suitably in place of large vessel cells even though there are morphological and functional differences between large vessel endothelial cells and microvascular endothelial cells in their native tissues.

Notwithstanding the work reported in this field, a need still exists for improved grafts, simple, reliable procedures which can successfully endothelialize the surfaces of human implants such as surfaces of vascular grafts, and for other methods of vascularization.

The present invention provides a solution to the observed problems by i) reducing the adsorption and activation of blood components that normally leads to activation of complement systems and coagulation systems, and ii) increasing the adhesive potential and growth potential for endothelial cells. Surface coating of blood-containing devices aimed at improving attachment and growth of endothelial cells and reducing attachment and activation of blood components represents one focus area of the present invention.

The present invention in one aspect provides a method for endothelialzing surfaces of human implants such as surfaces of vascular grafts such as blood contacting devices including small diameter blood vessels, and other methods of vascularization. The vascular grafts comprise at least a surface area comprising or essentially consisting of a material according to the invention comprising a substratum according to the invention.

Artificial blood vessels treated with the present invention can initially be seeded in vitro with endothelial cells derived from the patient to be treated, and then implanted subject to a pre-confluent coverage with endothelial cells. The natural environment in the blood vessel, including shear stress arising from flowing blood, provides adequate conditions for endothelial cell proliferation and correct functional activity. Also, any remaining cell free areas of the implant would have a reduced thrombogenic potential and would reduce the risk of thrombosis for the patient

Implantable Devices and Methods for Tissue Engineering

Another major focus area is concerned with providing implantable devices having improved healing properties and a reduced potential for inflammation. The implantable devices may be provided by means of tissue engineering.

Tissue engineering is a multidisciplinary science that utilizes basic principles from the life sciences and engineering sciences to create cellular constructs for transplantation. The first attempts to culture cells on a matrix for use as artificial skin, which requires formation of a thin three dimensional structure, has been described by Yannas and Bell (See, for example, U.S. Pat. Nos. 4,060,081, 4,485,097 and 4,458,678). They used collagen type structures which were seeded with cells, then placed over the denuded area. One problem with the use of the collagen matrices was that the rate of degradation is not well controlled. Another problem was that cells implanted into the interior of thick pieces of the collagen matrix failed to survive.

U.S. Pat. No. 4,520,821 to Schmidt describes the use of synthetic polymeric meshes to form linings to repair defects in the urinary tract. Epithelial cells were implanted onto the synthetic matrices, which formed a new tubular lining as the matrix degraded. The matrix served a two fold purpose—to retain liquid while the cells replicated, and to hold and guide the cells as they replicated.

In European Patent Application No. 88900726.6 “Chimeric Neomorphogenesis of Organs by Controlled Cellular Implantation. Using Artificial Matrices” by Children's Hospital Center Corporation and Massachusetts Institute of Technology, a method of culturing, dissociated cells on biocompatible, biodegradable matrices for subsequent implantation into the body was described. This method was designed to overcome a major problem with previous attempts to culture cells to form three dimensional structures having a diameter of greater than that of skin.

Vacanti and Langer recognized that there was a need to have two elements in any matrix used to form organs:
i) Adequate structure and surface area to implant a large volume of cells into the body to replace lost function, and
ii) A matrix formed in a way that will allow adequate diffusion of gases and nutrients throughout the matrix as cells attach and grow to maintain viability in the absence of vascularization.

Once implanted and vascularized, the porosity required for diffusion of the nutrients and gases was no longer critical.

To overcome some of the limitations inherent in the design of the porous structures which support cell growth throughout the matrix solely by diffusion, WO 93/08850 “Prevascularized Polymeric Implants for Organ Transplantation” by Massachusetts Institute of Technology and Children's Medical Center Corporation disclosed implantation of relatively rigid, non-compressible porous matrices which are allowed to become vascularized, then seeded with cells. It was difficult to control the extent of ingrowth of fibrous tissue, however, and to obtain uniform distribution of cells throughout the matrix when they were subsequently injected into the matrix.

Many tissues have now been engineered using these methods, including connective tissue such as bone and cartilage, as well as soft tissue such as hepatocytes, intestine, endothelium, and specific structures, such as ureters.

There remains a need to improve the characteristic mechanical and physical properties of the resulting tissues, which in some cases does not possess the requisite strength and pliability to perform its necessary fiction in vivo. Examples of particular structures include heart valves and blood vessels.

Despite major advances in its treatment over the past thirty-five years, valvular heart disease is still a major cause of morbidity and mortality in the United States. Each year 10,000 Americans die as a direct result of this problem. Valve replacement is the state-of-the art therapy for end-stage valve disease. Heart valve replacement with either nonliving xenografts or mechanical protheses is an effective therapy for valvular heart disease. However, both types of heart valve replacements have limitations, including finite durability, foreign body reaction or rejection and the inability of the non-living structures to grow, repair and remodel, as well as the necessity of life-long anticoagulation for the mechanical prothesis. The construction of a tissue engineered living heart valve could eliminate these problems.

Atherosclerosis and cardiovascular disease are also major causes of morbidity and mortality. More than 925,000 Americans died from heart and blood vessels disease in 1992, and an estimated 468,000 coronary artery bypass surgeries were performed on 393,000 patients. This does not include bypass procedures for peripheral vascular disease.

Currently, internal mammary and saphenous vein grafts are the most frequently used native grafts for coronary bypass surgery. However, with triple and quadruple bypasses and often the need for repeat bypass procedures, sufficient native vein grafts can be a problem. Surgeons must frequently look for vessels other than the internal mammary and saphenous vessels. While large diameter (0.5 mm internal diameter) vascular grafts of dacron or polytetraflorethylene (PTFE) have been successful, small caliber synthetic vascular grafts frequently do not remain patent over time. Tissue engineered blood vessels may off er a substitute for small caliber vessels for bypass surgery and replacement of diseased vessels.

It is therefore an object of the present invention to provide a method for making tissue engineered constructs which have improved mechanical strength and flexibility while at the same time retaining biocompatible properties of the materials being used.

It is a further object of the present invention to provide a method and materials for making valves and vessels which can withstand repeated stress and strain.

It is another object of the present invention to provide a method improving yields of engineered tissues following implantation.

It is yet another object of the present invention to provide polymer-based drug release systems, in particularly systems based on implantable materials.

Many state of the art biomaterials do not have a satisfactory degree of biocompatibility when being in contact with body fluids (e.g. blood) or body tissue. Polymeric surfaces attract neutrophils or mononuclear cells tying to phagocyte the material. This leads among others to the generation of oxygen radicals, and proteases subsequently start degrading the material and surrounding tissues. The cells also release a number of cytokines that attract other types of immune cells and provoke the approach of fibroblasts. This bio-incompatibility can finally lead to the formation of a fibrous capsule with all the signs of inflammatory processes. The consequence often is that the implant must be retrieved from the patient. The bio-incompatibility interferes negatively with the function of the implanted device in terms of permeability, mechanical stability, attachment, etc.

A primary reason for the inflammatory reactions is the adsorption of proteins from the surrounding liquids, including immunglobulins, complement factors and other factors, and the subsequent conformational changes of such factors that again provide the basis for attachment and activation of immune cells. Graft rejections are often observed.

Several attempts have been made to modify the surfaces of biomaterials in order to suppress inflammatory reactions. Most approaches have aimed at reducing the extent of adsorption of proteins. One substance used for this purpose is PEG, either attached via adsorption, such as Pluronic F127, or covalently bound via photoactivation or other chemistry.

Several attempts have also been made to immobilise very short PEGs (so-called glymes) by plasma deposition techniques. All these methods try to have a maximum concentration of PEG on the surface in order to promote a reduction of in vivo inflammatory responses.

Biocompatible materials comprising a substratum modified according to the invention and having an intermediate surface concentration of macromolecules immobilised onto the substratum is provided by means of the present invention and greatly diminish the inflammatory potential of the employed polymeric materials.

Accordingly, one embodiment of the present invention relates to an implantable prosthetic device, or an implant in general, for implantation into a vertebrate including a human or an animal, said device comprising a biocompaticle material according to the present invention. In another embodiment there is provided a synthetic implant such as a vascular graft commonly used to replace the large veins or arteries of human patients.

State of the art implants and implantable prosthetic devices having surfaces capable of being modified according to the present invention are described e.g. in U.S. Pat. No. 5,628,781 and U.S. Pat. No. 5,855,610.

The terms “implant”, “implantable device”, “implantation”, “implantable prothetic device”, “polymeric matrix”, and the like, as used herein, denotes an implant comprising a substratum having a surface capable of being modified according to the present invention shall denote: Any modification of a substratum as defined herein when said substratum is contacted by a macromolecule, is a contacting capable of generating: A biocompatible material comprising a substratum contacted by at least one macromolecule,
said material having a first advancing contact angle a,
said substratum having a second advancing contact angle b₀ when not contacted by a macromolecule, and another second advancing contact angle bat when said substratum is saturated by said macromolecules,
wherein said advancing contact angles are measured using water and air saturated by water vapour,
wherein bₛₐₜ essentially does not change when the substratum is contacted by further macromolecules by means of a chemical bond,
wherein the relation between said advancing contact angles is as defined by the ratio R,
   R=(b₀−a)/(b₀−bₛₐₜ)
and wherein the numerical value of R is in the interval from 0 to less than 0.4.

The material according to this aspect of the invention the invention may also be characterised as a biocompatible material comprising a substratum, wherein the material is generated by modifying the substratum by contacting the substratum with a macromolecule,
wherein said substratum is contactable with a macromolecule,
wherein said material further comprises at least one macromolecule,
wherein said material has a first contact angle a,
wherein said substratum has a second contact angle b₀ when not contacted by a macromolecule, and
wherein said contact angle a is substantially identical to said contact angle b₀.

In one embodiment of this aspect the invention provides improved yields of engineered tissue following implantation. The tissue optionally also has an enhanced mechanical strength and/or flexibility and/or pliability, and can be obtained by implantation, preferably subcutaneously, of a fibrous polymeric matrix comprising a biocompatible material according to the invention for a period of time sufficient to obtain ingrowth of fibrous tissue and/or blood vessels. The polymeric matrix is optionally seeded prior to a first implantation, after ingrowth of the fibrous tissue, or at the time of a reimplantation. The time required for fibrous ingrowth typically ranges from days to weeks. The method according to the invention is particularly useful in making valves and tubular structures, especially heart valves and blood vessels.

As described herein, biomaterials are created by seeding of fibrous or porous polymeric matrices with dissociated cells that are useful for a variety of applications, ranging from soft tissues formed of parenchymal cells such as hepatocytes, to tissues having structural elements such as heart valves and blood vessels, to cartilage and bone. In a particular improvement over the prior art methods, the polymeric matrices are initially implanted into a human or animal to allow a first ingrowth of fibroblastic tissue, and then implanted at the site where the structure is needed, either alone or seeded with defined cell populations.

Matrix Fabrication

The invention in one aspect provides a synthetic matrix that serves several purposes. It functions as a cell delivery system that enables the organized transplantation of large numbers of cells into the body. The matrix according to the invention acts as a scaffold providing three-dimensional space for cell growth. The matrix functions as a template providing structural cues for tissue development. In the case of tissues have specific requirements for structure and mechanical strength, the polymer temporarily provides the biomechanical properties of the final construct, giving the cells time to lay down their own extracellular matrix which ultimately is responsible for the biomechanical profile of the construct. The scaffold also determines the limits of tissue growth and thereby determines the ultimate shape of tissue engineered construct. Cells implanted on a matrix proliferate only to the edges of the matrix; not beyond.

Matrix Architecture

As previously described, for a tissue to be constructed, successfully implanted, and function, the matrices must have sufficient surface area and exposure to nutrients such that cellular growth and differentiation can occur prior to the ingrowth of blood vessels following implantation. This is not a limiting feature where the matrix is implanted and ingrowth of tissue from the body occurs, prior to seeding of the matrix with dissociated cells.

The organization of the tissue may be regulated by the microstructure of the matrix. Specific pore sizes and structures may be utilized to control the pattern and extent of fibrovascular tissue ingrowth from the host, as well as the organization of the implanted cells. The surface geometry and chemistry of the matrix may be regulated to control the adhesion, organization; and function of implanted cells or host cells.

In the preferred embodiment, the matrix is formed of polymers having a fibrous structure which has sufficient interstitial spacing to allow for free diffusion of nutrients and gases to cells attached to the matrix surface. This spacing is typically in the range of 100 to 300 microns, although closer spacings can be used if the matrix is implanted, blood vessels allowed to infiltrate the mat then the cells are seeded into the matrix. As used herein, “fibrous” includes one or more fibers that is entwined with itself, multiple fibers in a woven or non-woven mesh, and sponge like devices.

The matrix should be a pliable, non-toxic, injectable porous template for vascular ingrowth. The pores should allow vascular ingrowth and the injection of cells in a desired density and region(s) of the matrix without damage to the cells. These are generally interconnected pores in the range of between approximately 100 and 300 microns. The matrix should be shaped to maximize surface area, to allow adequate diffusion of nutrients and growth factors to the cells and to allow the ingrowth of new blood vessels and connective tissue.

The overall, or external, matrix configuration is dependent on the tissue which is to reconstructed or augmented. The shape can also be obtained using struts, as described below, to impart resistance to mechanical forces and thereby yield the desired shape. Examples include heart valve “leaflets” and tubes.

Substrata Capable of being Modified by a Macromolecule

The term “bioerodible”, or “biodegrable”, as used herein refers to materials which are enzymatically or chemically degraded in vivo into simpler chemical species. Either natural or synthetic polymer substrata, hereinafter simply denoted “polymers”, can be used to form the matrix. For some embodiments synthetic biodegradable polymers, optionally pretreated synthetic biodegradable polymers, are preferred for reproducibility and controlled release kinetics, whereas for other embodiments, synthetic non-biodegradable polymers, including pretreated, synthetic non-biodegradable polymers are preferred.

All polymers for use in the matrix must meet the mechanical and biochemical parameters necessary to provide adequate support for the cells with subsequent growth and proliferation. The polymers can be characterized with respect to mechanical properties such as tensile strength using an Instron tester, for polymer molecular weight by gel permeation chromatography (GPC), glass transition temperature by differential scanning calorimetry (DSC) and bond structure by infrared (IR) spectroscopy, with respect to toxicology by initial screening tests involving Ames assays and in vitro teratogenicity assays, and implantation studies in animals for immunogenicity, inflammation, release and degradation studies.

Polymer Coatings

In some embodiments, attachment of cells to the modified polymer substratum may optionally be enhanced even further by coating the polymers with compounds such as basement membrane components, agar, agarose, gelatin, gum arabic, collagens types I, II, IV, and V, fibronectin, laminin, glycosaminoglycans, polyvinyl alcohol, mixtures thereof, and other hydrophilic and peptide attachment materials known to those skilled in the art of cell culture. One material suitable for coating the polymeric matrix is polyvinyl alcohol or collagen.

Struts

In some embodiments it may be desirable to create additional structure using devices provided for support, referred to herein as “struts”. These can be biodegradable or nondegradable polymers which are inserted to form a more defined shape than is obtained using the cell-matrices. An analogy can be made to a corset, with the struts acting as “stays” to push the surrounding tissue and skin up and away from the implanted cells. In a preferred embodiment, the struts are implanted prior to or at the time of implantation of the cell-matrix structure. The struts are formed of a material comprising a modified polymeric substratum of the same type as can be used to form the matrix, as listed above, having sufficient strength to resist the necessary mechanical forces.

Additives to Polymer Matrices

In some embodiments it may be desirable to add bioactive molecules to the cells. A variety of bioactive molecules can be delivered using the matrices described herein. These are referred to generically herein as “factors” or “bioactive factors”.

In the preferred embodiment, the bioactive factors are growth factors, angiogenic factors, compounds selectively inhibiting ingrowth of fibroblast tissue such as anti-inflammatories, and compounds selectively inhibiting growth and proliferation of transformed (cancerous) cells. These factors may be utilized to control the growth and function of implanted cells, the ingrowth of blood vessels into the forming tissue, and/or the deposition and organization of fibrous tissue around the implant.

Examples of growth factors include heparin binding growth factor (hbgf), transforming growth factor alpha or beta (TGF-beta), alpha fibroblastic growth factor (FGF), epidermal growth factor (TGF), vascular endothelium growth factor (VEGF), some of which are also angiogenic factors. Other factors include hormones such as insulin, glucagon, and estrogen. In some embodiments it may be desirable to incorporate factors such as nerve growth factor (NGF) or muscle morphogenic factor (MM).

Steroidal antiinflammatories can be used to decrease inflammation to the implanted matrix, thereby decreasing the amount of fibroblast tissue growing into the matrix.

These factors are known to those skilled in the art and are available commercially or described in the literature. In vivo dosages are calculated based on in vitro release studies in cell culture; an effective dosage is that dosage which increases cell proliferation or survival as compared with controls, as described in more detail in the following examples. Preferably, the bioactive factors are incorporated to between one and 30%0 by weight, although the factors can be incorporated to a weight percentage between 0.01 and 95 weight percentage.

Bioactive molecules can be incorporated into the matrix and released over time by diffusion and/or degradation of the matrix, they can be suspended with the cell suspension, they can be incorporated into microspheres which are suspended with the cells or attached to or incorporated within the matrix, or some combination thereof. Microspheres would typically be formed of materials similar to those forming the matrix, selected for their release properties rather than structural properties. Release properties can also be determined by the size and physical characteristics of the microspheres.

Cells to Be Implanted

Cells to be implanted are dissociated using standard techniques such as digestion with a collagenase, trypsin or other protease solution. Preferred cell types are mesenchymal cells, especially smooth or skeletal muscle cells, myocytes (muscle stem cells), fibroblasts, chondrocytes, adipocytes, fibromyoblasts, and ectodermal cells, including ductile and skin cells, hepatocytes, Islet cells, cells present in the intestine, and other parenchymal cells, osteoblasts and other cells forming bone or cartilage. In some cases it may also be desirable to include nerve cells. Cells can be normal or genetically engineered to provide additional or normal function. Methods for genetically engineering cells with retroviral vectors, polyethylene glycol, or other methods known to those skilled in the art can be used.

Cells are preferably autologous cells, obtained by biopsy and expanded in culture, although cells from close relatives or other donors of the same species may be used with appropriate immunosuppression. Immunologically inert cells, such as embryonic or fetal cells, stem cells, and cells genetically engineered to avoid the need for immunosuppression can also be used. Methods and drugs for immunosuppression are known to those skilled in the art of transplantation. A preferred compound is cyclosporin using the recommended dosages.

Cells to be implanted can also be derived from blood or from bone marrow from which it is possible to isolate adult pluripotent stem or precursor cells. Particularly stem cells from bone marrow have a broad range of applicability to be differentiated in direction of blood cells, such as leukocytes or chondrocytes (cartilage) or osteoblasts depending on the culture conditions including nutrients and growth factors.

In the preferred embodiment, cells are obtained by biopsy and expanded in culture for subsequent implantation. Cells can be easily obtained through a biopsy anywhere in the body, for example, skeletal muscle biopsies can be obtained easily from the arm, forearm, or lower extremities, and smooth muscle can be obtained from the area adjacent to the subcutaneous tissue throughout the body. To obtain either type of muscle, the area to be biopsied can be locally anesthetized with a small amount of lidocaine injected subcutaneously. Alternatively, a small patch of lidocaine jelly can be applied over the area to be biopsied and left in place for a period of 5 to 20 minutes, prior to obtaining biopsy specimen. The biopsy can be effortlessly obtained with the use of a biopsy needle, a rapid action needle which makes the procedure extremely simple and almost painless. With the addition of the anesthetic agent, the procedure would be entirely painless. This small biopsy core of either skeletal or smooth muscle can then be transferred to media consisting of phosphate buffered saline. The biopsy specimen is then transferred to the lab where the muscle can be grown utilizing the explant technique, wherein the muscle is divided into very pieces which are adhered to culture plate, and serum containing media is added. Alternatively, the muscle biopsy can be enzymatically digested with agents such as trypsin and the cells dispersed in a culture plate with any of the routinely used medias. After cell expansion within the culture plate, the cells can be easily passaged utilizing the usual technique until an adequate number of cell is achieved.

Methods for Implantation

Unlike other prior art methods for making implantable matrices, the present method uses the recipient or an animal as the initial bioreactor to form a fibrous tissue polymeric construct which optionally can be seeded with other cells and implanted. The implanted matrix becomes infiltrated with fibrous tissue and/or blood vessels over a period ranging from between one day and a few weeks, most preferably one and two weeks. The implanted matrix according to the invention is then removed and implanted at the site where it is needed

In one embodiment, the matrix is formed of polymer fibers having a particular desired shape, that is implanted subcutaneously. The implant is retrieved surgically, then one or more defined cell types distributed onto and, into the fibers. In a second embodiment, the matrix is seeded with cells of a defined type, implanted until fibrous tissue has grown into the matrix, then the matrix removed, optionally cultured further in vitro, then reimplanted at a desired site.

The resulting structures are dictated by the matrix construction, including architecture, porosity (% void volume and pore diameter), polymer nature including composition, crystallinity, molecular weight, and degradability, hydrophobicity, and the inclusion of other biologically active molecules.

This methodology is particularly well suited for the construction of valves and tubular structures. Examples of valves are heart valves and valves of the type used for ventricular shunts for treatment of hydrocephaly. A similar structure could be used for an ascites shunt in the abdomen where needed due to liver disease or in the case of a lymphatic obstructive disease. Examples of tubular structures include blood vessels, intestine, ureters, and fallopian tubes.

The structures are formed at a site other than where they are ultimately required. This is particularly important in the case of tubular structures and valves, where integrity to fluid is essential, and where the structure is subjected to repeated stress and strain.

The present invention in one preferred embodiment pertains to a tissue engineered heart valve comprising an implanted device in the form of a matrix comprising a substratum that has been modified according to the invention by contacting the substratum with a macromolecule.

Valvular heart disease is a significant cause of morbidity and mortality. Construction of a tissue engineered valve using living autologous cells offers advantages over currently used mechanical or glutaraldehyde fixed xenograft valves.

A tissue engineered valve can be constructed by seeding a material according to the invention comprising a substratum contacted by a macromolecule with dissociated fibroblasts and endothelial cells harvested from a donor heart valve, including an animal including human donar heart valve.

As an example, current lab technology in culturing blood vessels and heart valves are based e.g. on the digestion of these items from pig for example remaining only non immunogenic collagens and elastins. Then by biopsy or harvesting from blood vessels autologous endothelial and smooth muscel cells are obtained, expended in vitro and then seeded onto a scaffold. The scaffold in one preferred embodiment of the present invention is a biocompatible material as disclosed herein comprising a preferably porous, bioerodable scaffold.

The present invention in another preferred embodiment pertains to tissue engineered vascular structures comprising an implanted device in the form of a matrix comprising a substratum contacted by a macromolecule.

Vascular smooth muscle tubular structures using a polymer scaffold comprising a biocompatible material according to the invention represent one such embodiment. This technique involves the isolation and culture of vascular smooth muscle cells, the reconstruction of a vascular wall using either a biodegradable polymer or a non-biodegradable polymer, and formation of the neo-tissue tubes in vitro. In one aspect of the invention there is provided vascular structures comprising a polymer matrix according to the invention, and a method for engineering vascular structures by coculturing endothelial cells with fibroblasts and smooth muscle cells on a modified substratum according to the invention in order to create tubular constructs that histologically resemble native vascular structures.

In a still further embodiment the present invention pertains to engineered bone from a polymer scaffold comprising a biocompatible material according to the invention and periosteum. The ability to create bone from periosteum and either a biodegradable polymer matrix or a non-biodegradable polymer matrix may have significant utility in reconstructive orthopedic and plastic surgery. The invention thus in one aspect provides new bone constructs formed from periosteum or periosteal cells, as well as from bone marrow derived bone precursor (stem) cells.

In yet another embodiment the present invention pertains to bone reconstruction with tissue engineering vascularized bone. The invention provides new vascularized bone engineered by transplantation of osteoblasts around existing vascular pedicle using either a biodegradable polymer matrix or a non-biodegradable polymer matrix as a cell delivery device in order to, reconstruct weight bearing bony defects.

In a still further embodiment there is provided a method of engineering composite bone and cartilage. The ability to construct a composite structure of bone and cartilage offers a significant modality in reconstructive plastic and orthopedic surgery. The invention thus provides a method for engineering of bone and cartilage composite structure using periosteum, chondrocytes and a modified substratum contacted by a macromolecule according to the invention in order to direct bone and cartilage formation by selectively placing periosteum and chondrocytes onto the polymer scaffold

In an even further embodiment there is provided a method for performing an implantation of a matrix polymer comprising a biocompatible material according to the invention comprising a modified substratum according to the invention for ingrowth of fibrous tissue to increase mechanical properties and cell survival. The aim of the method is to increase the mechanical strength and pliability of e.g. heart valve leaflets and other engineered tissues such as those for use as artificial blood vessels while at the same time retaining the biocompatibility of a polymer matrix comprising a biocompatible material according to the invention.

The present invention in another embodiment provides improved implants comprising a biocompatible material according to the invention that facilitates deposition of endothelial cells in suspension and reduce the inherent thrombogenicity of the implants. Improved methods of preparing endothelialized implants according to the present invention are also provided. The term “implant” as used herein below will denote an implant or implantable device comprising a biocompatible material according to the present invention.

In some embodiments of the present invention, improved implants have porosity sufficient to allow the surface of the implants to be used as filters. Endothelial cells may be deposited in pores of implants in other aspects of the invention.

The present invention in one embodiment has the general objective of improving vascular implants. Earlier work was aimed at either: (1) developing implants with an artificial, non-thrombogenic surface, or (2) lining vascular prostheses with human endothelial cells, in the hope of producing a non-thrombogenic endothelial cell surface such as exists in native human vessels.

Implants encompassed by the present invention include, but are not limited to, for example, intravascular devices such as artificial vascular prostheses, artificial hearts, and heart valves. It is anticipated that the herein described procedures may lead to the development of other artificial organs or devices. These organs and devices will receive circulating blood either following implantation or in an extracorporeal circuit, and the present procedures provide a non-thrombogenic or anti-thrombogenic interface between the blood and the implanted surface.

In some embodiments of the present invention novel implants may be made from implant material having porosity. Such porosity may provide a filtering function, facilitating deposition of cells suspended in aqueous solution on the implant surface and within the pores of the implant. Furthermore, cells deposited within the pores of the implant by deposition, or by other processes, may not initially be in contact with blood flow, thus reducing thrombogenicity of the implant.

The implants may be comprise any polymeric substratum contacted by a macromolecule or biocompatible material according to the invention ranging in porosity from about 0.1 to about 100 microns, preferably ranging from about 1 to about 50 microns, such as from about 2 to about 25 microns. For example, implant material can be a polymer such as polyester polytetrafluoroethylene, or a naturally occurring material such as an umbilical vein, saphenous vein, or native bovine artery.

It is preferable in some aspects of the present invention to optimize water flow through characteristics. It is known from U.S. Pat. No. 5,628,781 that the deposition of endothelial cells onto the surface of implant material is increased when the implant has significant water flow through characteristics. Implants having flow through characteristics useful to allow the surface of the implant to be used as a filter generally having porosity of from about 1 to about 4 microns.

Optimally, an implant according to the present invention will have a permeability of at least about 10 ml/min/cm². In preferred embodiments of the present invention, permeability will range from about 10 ml/min/cm² to about 40 ml/min/cm². Pore coverage may optimally be at least about 8%. In preferred embodiments of the present invention, pore coverage is from about 12% to about 16%. In other embodiments, implants may have at least some porosity of from about 10 to about 20 microns in which endothelial cells may be deposited.

The implants comprising a biocompatible material according to the present invention in one embodiment results in endothelial cells exhibiting a reduced thrombogenicity because of a much improved contact between the cells and different matrix proteins of the basement membrane. This is important as shown by e.g. Madri and Williams (J. Cell Biol. 1983, 97, 153) demonstrating that growth of endothelial cells was reduced when cells were placed on-surfaces containing type IV/V collagen, the surface cells normally reside on, as compared to e.g. type I/III collagen.

In some embodiments of the present invention, an implant material such as any commercially available polymer implant material capable of being modified according to the present invention, may be treated by glow-discharge plasma modification to provide a surface having properties similar to basement membrane. For example, polyurethane vascular grafts-may be modified by a pretreatment including corona treatment and plasma treatment as described herein, including modification by glow-discharge plasma (Plastics, 85, Proceedings of the SPE 43rd Annual Technical Conference and Exhibition pp. 685-688 (1985)) using e.g. tubular geometric technology of the Becton-Dickinson Company (Franklin Lakes, N.J.) to produce a surface chemistry on the inside of a tubular graft which is similar to basement membrane.

Thus, implants according to the present invention may optionally be subjected to a pretreatment comprising e.g. glow-discharge plasma treated prior to being modified according to the present invention. The implants may also have a predetermined porosity to enhance adherence of endothelial cells and reduce thrombogenicity.

In some embodiments of the present invention, the material comprising a suitable porous implant substratum that has been modified as described herein, and optionally pretreated including glow-discharge plasma treated, may be useful as an implant such as a vascular graft. In such embodiments of the present invention, endothelial cells are deposited on the surface and/or within the pores of the porous implant material by means of e.g. a filtration action wherein an aqueous phase containing endothelial cells is passed through the porous implant, leaving behind cells deposited on the surface and/or in the pores below the lumenal surface of the implant.

Cell adherence to the surface and within the pores of the implant will be enhanced by the using implant materials pertaining to the present invention comprising a biocompatible substratum modified according to the invention with a macromolecule suitable for modifying the substratum in accordance with the desired objective for use of the material in question. In some embodiments of the present invention, the surface of the substratum contacted by a macromolecule forming the vascular graft may initially be treated with a surfactant or cleaning agent to make it more easily wettable.

Endothelial cells suspended in an aqueous phase may be microvascular endothelial cells isolated and prepared by any state of the art method including the method described in e.g. Ser. No. 725,950, filed Jun. 27, 1991, and incorporated by reference herein in its entirety. Endothelial cells may be deposited on the implant by suspending the isolated endothelial cells in a buffered saline which contains plasma-derived protein from the patient. The protein solution is prepared by mixing six parts buffered solution with one part plasma to produce a solution which contains approximately one percent (1%) protein. Albumin is the preferred source of the protein, but non-plasma sources of protein can be used. The microvascular endothelial cell suspension is then preferably pelletized by centrifugation (200.times.g) and the pellet resuspended with protein containing buffer solution. This resuspension should be performed at a ratio of approximately 1:5 to 1:15 or about 1:10 volumes of packed microvascular endothelial cells to buffer solution. The cell suspension is filtered through the surface to provide a layer of endothelial cells on the surface and within the pores of the implant to be treated. Time needed for adherence of the cells to the surface and within the pores of the implant comprising a substratum modified in accordance with the present invention will vary depending upon the implant material and any pretreatments the implant may have received. For example, endothelial cells will adhere to an untreated polyester graft surface in two hours, while pretreatment of the polyester graft with protein will generally tend to reduce the time for adherence. Following incubation for a sufficient time, the implant may be washed with a protein containing buffer, and the washed implant may now be implanted.

The porous implant material according to the invention may also be useful to provide vascularization without the use of a vascular graft. In such embodiments, implant material is treated with endothelial cells by filtration or by simple deposition such that the endothelial cells are deposited within the pores of the implant material as described above and the implant is implanted in a normal manner. Vascularization is accomplished by engrowth of surrounding endothelial cells with transplanted cells from the new vascular conduit.

In some, embodiments of the present invention, endothelial cells deposited in the pores of the implant may be transformed to have desired biological properties. For example, said endothelial cells may be transformed with a gene for a heterologous protein useful as a therapeutic agent, such as a gene coding for plasminogen activator, soluble CD-4, Factor VIII, Factor IX, von Willebrand Factor, urokinase, hirudin, interferons, tumor necrosis factor, interleukins, hematopoietic growth factor, antibodies, glucocerebrosidase, ADA, phenylalanine, hydroxylase, human growth hormone, insulin and erythropoietin. Endothelial cells may also be transformed by nucleic acids coding for therapeutic agents by methods known to those skilled in the art. Nucleic acids as used herein denote the common meaning of the word, i.e. a DNA or RNA sequence which encodes a functional protein or RNA molecule. Genes of the present invention may be synthetic or naturally occurring.

Tranformation is the process by which cells have incorporated an exogenous gene by direct infection, transfection or other means of uptake. In preferred embodiments of the present invention, transformation is accomplished by means of a liposome-mediated transfection as described in Ausubel, et al., Current Protocols in Molecular Biology (1991) incorporated by reference herein in its entirety. A gene coding for a therapeutic agent is incorporated into a suitable vector such as pSG5 (Stratagene Cloning Systems, La Jolla, Calif.). Other vectors having characteristics useful in the present invention will be apparent to those skilled in the art. The term “vector” is well understood in the art and is synonymous with the phrase “cloning vehicle”. A vector carrying one or more desired genes may be used to transform endothelial cells of the present invention by standard procedures known in the art.

According to one preferred embodiment of the invention, surface functionalization is mediated by well-defined photo-reactive conjugates of hydrophilic, flexible macromolecules comprising a modular composition of building blocks. However, other forms of attachment besides photo activation can also be used.

In one particularly preferred embodiment said modular composition comprises:

Latent-Reactive Head-Group)-(Guiding-Group)-(Main Body)-(Functional End-Group)

In another embodiment, the guiding group is optional and the macromolecule comprises only a latent-reactive head group, a main body, and a functional end group, and no guiding group. A linker group can optionally also be present.

The invention aims to provide a substratum surface with desired physical characteristics and comprises the steps of contacting the substratum with a composition comprising a plurality of macromolecules possessing desired physical characteristics. The macromolecules each comprise covalently bonded, optionally via a linker group, to their main body, a latent-reactive head-group, and optionally also a guiding group, and a functional end-group. The latent-reactive head-group is capable of providing one or more active species such as free radicals in response to external stimulation to covalently bind the macromolecules to the substratum, through the residues of the latent-reactive head-group.

The macromolecule is spatially oriented so as to enable one or more of its latent-reactive groups to come into covalent bonding proximity with the substratum surface, and the method according to the present invention includes the further step of activating the latent-reactive groups by applying external stimulation to covalently bond the macromolecule to the substratum. The external stimulation that is employed is preferably electromagnetic radiation, and more preferably the radiation is in the ultraviolet, visible or infra-red regions of the electromagnetic spectrum, since the layer structure established by “self-assembly” is not disturbed by this kind of radiation, and the polymer substratum is left at least substantially intact. The degree of conversion is selectable by e.g. UV/Vis dose, and typically 100% conversion will be attempted. The response to the activation step of the method can be tuned by selecting different latent-reactive groups. Also, the reactivity of the photo-chemically generated reactive species can be selected in accordance to the structure of the polymer substratum. Thus, it is well known that e.g. aryl nitrenes from aryl azides will react via insertion reactions with all polymers having —NH, —OH or —CH groups, and aromatic ketones after UV/Vis excitation will undergo a hydrogen abstraction eventually leading to an insertion reaction with all polymers having at least —CH groups

The latent-reactive head-group of a macromolecule employed in the invention may comprise one or more covalently bonded latent-reactive groups. The latent-reactive groups, as defined herein, are groups which respond to specific applied external stimuli to undergo an active species generation resulting in covalent bonding to an adjacent support surface. Latent-reactive groups are those groups of atoms in a molecule which retain their covalent bonds unchanged under conditions of storage but which, upon activation, form covalent bonds with other molecules. The latent-reactive groups generate active species such as free radicals, nitrenes, carbenes, and excited states of ketones upon absorption of external electromagnetic or kinetic (thermal) energy. Latent-reactive groups may be chosen to be responsive to various portions of the electromagnetic spectrum, and latent-reactive groups that are responsive to ultraviolet, visible or infrared portions of the spectrum are preferred.

The azides constitute a preferred class of latent-reactive groups and include arylazides such as phenyl azide, 4-azido benzoic acid, and 4-fluoro-3-nitrophenyl azide, acyl azides such as benzoyl azide and p-methylbenzoyl azide, azido formates such as ethyl azidoformate, phenyl azidoformate, sulfonyl azides such as benzenesulfonyl azide, and phosphoryl azides such as diphenyl phosphoryl azide and diethyl phosphoryl azide. Diazo compounds constitute another class of latent reactive groups and include diazoalkanes (—CHN₂) such as diazomethane and diphenyldiazomethane diazoketones such as diazoacetophenone and 1-trifluoromethyl-1-diazo-2-pentanone, diazoacetates such as t-butyl diazoacetate and phenyl diazoacetate, and beta-keto-alpha-diazoacetates such as t-butyl alpha diazoacetoacetate. Other latent-reactive groups include the aliphatic azo compounds such as azobisisobutyronitrile, the diazirines such as 3-trifluoromethyl-3-phenyldiazirine, the ketenes (—CH═C═O) such as ketene and diphenylketene and photoactivatable ketones such as benzophenone and acetophenone. Peroxy compounds are contemplated as another class of latent-reactive groups and include dialkyl peroxides such as di-t-butyl peroxide and dicyclohexyl peroxide and diacyl peroxides such as dibenzoyl peroxide and diacetyl peroxide and peroxyesters such as ethyl peroxybenzoate.

Upon activation of the latent-reactive groups to cause covalent bond formation to the surfaces to which macromolecules are to be attached, the macromoleculesare covalently attached to the surfaces by means of residues of the latent reactive groups.

As will be noted from the foregoing disclosure, photoreactive groups are for the most part aromatic and are hence generally hydrophobic rather than hydrophilic in nature. The presence of a comparatively hydrophobic reactive head-group such as an aromatic photoreactive group, appears to be causing the macromolecule to orient itself in an aqueous solution with respect to a hydrophobic substratum surface such that the comparatively hydrophobic reactive head-group is preferentially carried near the support surface while the remainder of the macromolecule, i.e. the main body and the functional end-group, is generally orientated away from the hydrophobic substratum surface. It is known that this feature enables macromolecules to be covalently bonded densely to a comparatively hydrophobic support substratum surface, and this in turn contributes to the formation of a biocompatible substratum surface as defined above.

According to the above the amphiphilic character and thus orientation and achieved grafting density of macromolecules to a substratum surface can be increased by incorporating a hydrophobic guiding-group into the macromolecule. The guiding-group is a bifunctional group that is positioned, preferably by means of a linker group, between the latent-reactive head-group and the remainder of the macromolecule, i.e. the main body and the functional end-group. The guiding-group is hydrophobic for the purpose of enhancing the preferential orientation of the latent-reactive head-group of the macromolecule into bonding proximity of the substratum surface and for the purpose of increasing the amphiphilic character of the macromolecule in order to increase the achieved grafting density. Preferred classes of guiding groups are aliphatic, linear or weakly branched groups or cyclic aliphatic groups, both preferably with from. 6 to 0.18 carbon atoms, or combinations thereof, as well as mono- or polycyclic aromatic groups, or their combinations with the above-mentioned aliphatic groups.

The main body of the macromolecule is preferably hydrophilic, uncoiling in an aqueous environment and thus exhibiting an excluded volume. It may be a polymer of natural or synthetic origin. Such polymers include oligomers, homopolymers and co-polymers resulting from addition or condensation polymerization, and natural polymers including oligosaccharides, polysaccharides, oligosaccharides, and polypeptides or a part thereof such as an extended oligopeptide. The polymer forming the main body may comprise several distinct polymer types, as prepared by terminal or side chain grafting, including cellulose-based products such as hydroxyethyl cellulose, hydroxypropyl cellulose, carboxymethyl cellulose, cellulose acetate and cellulose butyrate, acrylics such as those polymerized from hydroxyethyl acrylate, hydroxyethyl methacrylate, glyceryl acrylate, glyceryl methacrylate, acrylic acid, methacrylic acid, acrylamide and methacrylamide, vinyls such as polyvinyl pyrrolidone and polyvinyl alcohol, nylons such as polycaprolactam, polylauryl lactam, polyhexamethylene adipamide and polyhexamethylene dodecanediamide; polyurethanes, polylactic acids, linear polysaccharides such as amylose, dextran, chitosan, and hyaluronic acid, and branched polysaccharides such as amylopectin, hyaluronic acid and hemi-celluloses.

The macromolecules themselves preferably have MWs of at least about 500 Da, most preferably of about 10.000. Da, and are hydrophilic in nature, and soluble in water to the extent of at least approximately 0.5% by weight at 25° C.

In a preferred embodiment the main body comprises repeating units as e.g. ethoxy (—CH₂—CH₂—O—) or isopropoxy (—CH₂—CH(CH₃)—O—) groups, and of these PEG is most preferred.

Functional endgroups include all chemical moieties that can be used to link permanently or reversibly other biological or synthetic molecules or cells, viruses and the like via the polymeric main body to a surface, such as hydroxy, amino, carboxyl, sulphonic acid, activated esters, or epoxy groups as well as charged or chelating functionalities.

Additionally, the functional end-group may be chosen from a wide variety of compounds or fragments thereof which will render the modified substratum generally or specifically “biophilic” as those terms are defined below. Generally biophilic functional end-groups are those that would generally promote the binding, adherence, or adsorption of biological materials such as, for example, intact cells, fractionated cells; cellular organelles, proteins, lipids, polysaccharides, simple carbohydrates, complex carbohydrates, and/or nucleic acids. Generally biophilic functional end-groups include hydrophobic groups or alkyl groups with charged moieties such as —COO⁻; —PO₃H⁻ or 2-imidazolo groups, and compounds or fragments of compounds such as extracellular matrix proteins; FN, collagen, laminin, serum albumin, polygalactose, sialic acid, and various lectin binding sugars. Specifically biophilic functional end-groups are those that-selectively or preferentially bind, adhere or adsorb a specific type or types of biological material so as, for example, to identify or isolate the specific material from a mixture of materials. Specific biophilic materials include antibodies or fragments of antibodies and their antigens, cell surface receptors and their ligands, nucleic acid sequences and many others that are known to those of ordinary skill in the art. The choice of an appropriate biophilic functional end-group depends on considerations of the biological material sought to be bound, the affinity of the binding required; availability facility of ease, and cost. Such a choice is within the knowledge, ability and discretion of one of ordinary skill in the art.

For the preparation of biodegradable coatings or coatings that may be degraded under predefined environmental conditions, it is desirable to incorporate in the macromolecule a moiety that allows either enzymatic or chemical hydrolysis of the coating. Suitable ingredients include amino acids such as alanine, valine, leucine, proline, methionine, aspartic acid, threonine, serine, glutamic acid, glycine, cysteine, phenylalanine, lysine, histidine, argine, and aminobutyric acid. Alternatively, hydrolytically unstable ester bonds can be applied as well. All these moieties are typically part of a linker group, when such a group is present, but may also be incorporated into the main-body or the guiding-group of the macromolecule.

The lateral density of the monolayer of macromolecules according to the invention is adjustable by e.g. i) modification of the amount and/or concentration of macromolecules in solution during “self-assembly”, or ii) the use of mixtures of macromolecules, said macromolecules comprising varying building blocks as e.g. different MWs (MW), or variations in other structural features of the macromolecule (e.g. branched vs. unbranched), or iii) adjustable by appropriately choosing solution conditions during an adsorptive application of said macromolecules, as e.g. the solvency, the ionic strength, the temperature or the pH. The process of photochemical grafting does neither disturb this “self-assembled” pattern, nor does it result in any substantial degradation of the underlying surface of the polymer substratum.

The substratum comprises a definable surface such as the tangible surface of film or a membrane, or the surface of a contact lens or surgical implant, or the surface provided by small particles in an emulsion or other suspension or as a powder, or as the surface of a soft gel. The invention provides the particular advantage of providing means by which non-pretreated definable (e.g., solid) surfaces may simply and rapidly be provided with covalently bonded macromolecular coatings in a simple, rapid and hence economical manner.

Preferred embodiments of the invention are described herein below. The material according to the invention may comprise soluble substance in the form of molecules capable of forming a self-assembled monolayer. Also, the substratum may be pretreated or modified, preferably as the result of said substratum being contacted by and/or operably linked to a charged group or a hydrophilic compound.

As defined above, the contact angle of said material is an advancing contact angle angle. In one embodiment, the advancing contact angle is in the range of from 50 degrees to 140 degrees, such as in the range of from 55 degrees to a 130degrees, preferably in the range of from 60 degrees to 125 degrees, such as in the range of from 70 degrees to 120 degrees, for example in the range of from 75 degrees to 110 degrees, such as in the range of from 80 degrees to 100 degrees, for example in the range of from 85 degrees to 95 degrees.

However, a material also exhibits a receding contact angle; in which case the contact angle is in the range of from 30 degrees to 120 degrees, preferably in the range of from 40 degrees to 110 degrees, such as in the range of from 50 degrees to 100 degrees, for example in the range of from 60 degrees to 90 degrees, such as in the range of from 70 degrees to 80 degrees.

The ratio between the difference between said second contact angle, when no macromolecule is present, and said first contact angle, and the difference between said second contact angle, when no macromolecule is present, and the contact angle of said substratum, when said substratum is saturated by said macromolecules as defined herein is more than −0.6 and less than 0.6, and preferably in the range of from 0 to less than a 0.50, such as less than 0.40, for example less than 0.30, such as less than 0.25, for example less than 0.20, such as less than 0.15, for example less than 0.110, such as less than 0.05.

When the contact angle is the receding contact angle the ratio is preferably less than 0.40.

The ratio between the difference between the third contact angle of said monolayer, when no macromolecule is present, and said first contact angle, and the difference between the third contact angle of said monolayer, when no macromolecule is present; and the contact angle of said self-assembled monolayer, when said monolayer is saturated by said macromolecules as defined herein, is more than −0.6 and less than 0.6, and preferably in the range of from 0 to less than 0.50, such as less than 0.40, for example less than 0.30, such as less than 0.25, for example less than 0.20, such as less than 0.15, for example less than 0.10, such as less than 0.05.

In one particularly preferred embodiment there is provided a material which, when contacted by a first determinant comprising a compound selected from the group consisting of a polypeptide, or part thereof, a nucleic acid moiety, a carbohydrate moiety, and a lipid moiety, including any combination thereof, is capable of maintaining said compound in a biologically active form. More preferably the compound is a polypeptide or part thereof.

There is also provided a material further comprising said first determinant comprising said compound, wherein said first determinant is maintained in a biologically active form when contacted by said substratum and/or said macromolecule. The biologically active form is preferably an essentially biologically active conformation. The biologically active form or conformation is preferably maintained and/or improved and/or stabilized by means of the cooperativity of said substratum and said macromolecule. The biologically active form or confirmation is preferably maintained and/or improved and/or stabilized when contacted by said substratum and said macromolecule. The material according to the invention is preferably biocompatible.

There is also provided a material according to the invention, wherein the weight increase per area unit arising from the part of the macromolecule essentially consisting of PEG or poly(ethylene oxide) (PEO) is less than 2.0×10⁻²² grams (g) per square nanometer (nm²), for example less than 1.8×10⁻²² grams (g) per square nanometer (nm²), such as less than 1.6×10⁻²² grams (g) per square nanometer (nm²), for example less than 1.4×10⁻²² grams (g) per square nanometer (nm²), such as less than 1.2×10⁻²² grams (g) per square nanometer (nm²), for example less than 1.0×10⁻²² grams (g) per square nanometer (nm²), for example less than 0.8×10⁻²² grams (g) per square nanometer (nm²), such as less than 0.5×10⁻²² grams (g) per square nanometer (nm²), for example less than 0.3×10⁻²² grams (g) per square nanometer (nm²) such as less than 0.2×10⁻²² grams (g) per square nanometer (nm²), for example less than 0.1×10⁻²² grams (g) per square nanometer (nm²).

Assuming a density of 1 gram/cm³, the above values correspond to a “layer” thickness of from less than 2 Å to less than 0.1 Å (i.e. 2 Å equals 2.0×10⁻²² grams (g) per square nanometer (nm²), and so forth).

There is also provided a material wherein the substratum is contacted by a plurality of soluble compounds capable of forming a layer of self-assembled macromolecules, preferably n-alkane chains preferably containing from 8 to 24 carbons. The macromolecule according to the invention can be characterized by an excluded volume.

The substratum preferably comprises a hydrophobic polymer and in one embodiment the substratum is at least substantially flexible and/or a film. However, the substratum may also be essentially rigid or at least substantially non-flexible. In this case, the substratum may comprise a crystalline structure capable of supporting a self-assembled monolayer such as gold, silicon oxide, and similar crystalline structures and/or structures that are smooth on a nanometer scale.

The macromolecule according to the invention comprises a hydrophilic polymer or an amphiphilic polymer. The macromolecule preferably has a MW of more than 400 Da, such as a MW of more than 1,000 Da, such as a MW of more than 2,000 kDa, for example a MW of more than 3,000 kDa, for example a MW of more than 4,000 kDa, for example a MW of more than 5,000 kDa, for example a MW of more than 6,000 kDa, for example a MW of more than 7,000 kDa, for example a MW of more than 8,000 kDa, for example a MW of more than 9,000 kDa, such as a MW of more than 10,000 Da, for example a MW of more than 12,000 kDa, for example a MW of more than 15,000 kDa, for example a MW of more than 20,000 kDa, for example a MW of more than 25,000 kDa, for example a MW of more than 50,000 Da, such as a MW of more than 100,000 Da.

The macromolecule according to the invention is preferably a conjugate comprising a head group, a guiding group, a linker group, a polymer chain or a main body, and a functional end group.

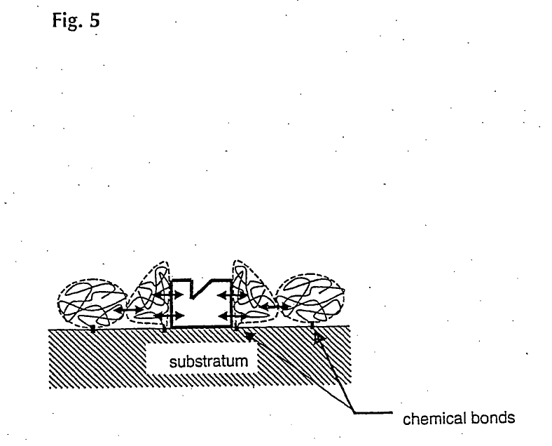

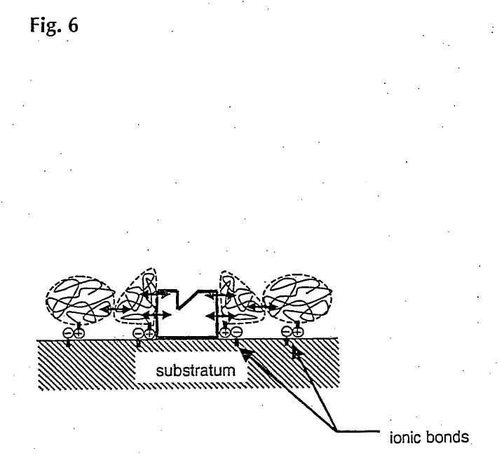

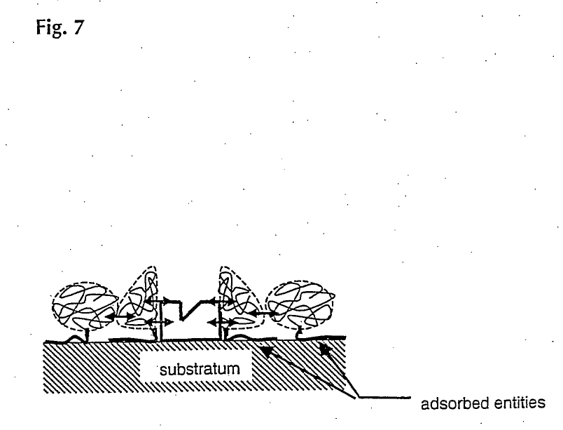

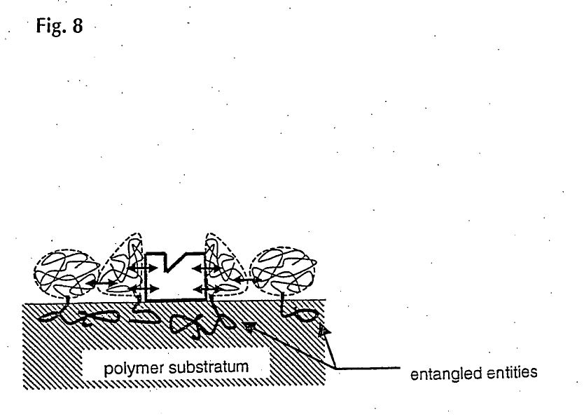

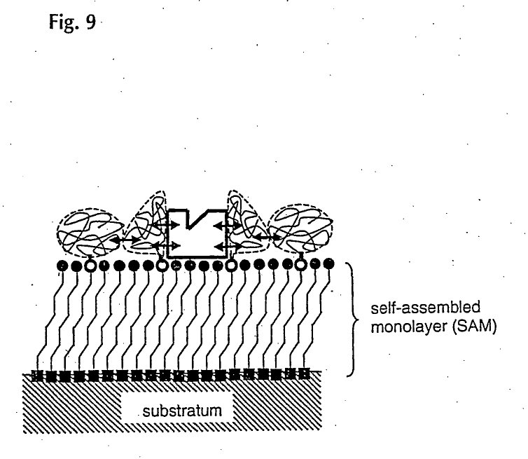

The head group is capable of forming a chemical bond (see FIG. 5), such as a ionic bond (see FIG. 6), and may adsorb to the substratum (see FIG. 7) or be entangled into or with the substratum (see FIG. 8). The head group may also be capable of forming a self-assembled monolayer (see FIG. 9).

A preferred guiding group is a bifunctional group comprising an aliphatic, linear or weakly branched group. The guiding group may also be capable of forming and/or stabilizing a self-assembled monolayer.

A preferred linker group is capable of being enzymatically or chemically hydrolyzed, it may be hydrolytically unstable, or it may be essentially stable against cleavage under practical circumstances.

The polymer chain or main body is preferably-hydrophilic, uncoiling in an aqueous environment and exhibiting an excluded volume.

The functional end group is capable of linking permanently or reversibly other biological or synthetic molecules or materials.

A first determinant as defined herein comprises a biologically active compound comprising a polypeptide, or a part thereof, a nucleic acid moiety, a carbohydrate moiety, and a lipid moiety, including any combination thereof. The biologically active compound is preferably selected from the group consisting of membrane associated and/or extracellular matrix polypeptides natively produced by a microbial cell, a plant cell or a mammalian cell. The biologically active compound in another embodiment is selected from the group consisting of a polypeptide, an antibody, a polyclonal antibody, a monoclonal antibody, an immunogenic determinant an antigenic determinant, a receptor, a receptor binding protein, an interleukine, a cytokine, a cellular differentiation factor, a cellular growth factor, and an antagonist to a receptor.

The biologically active compound may also be a synthetic polypeptide, or part thereof, capable of contacting said substratum and/or said macromolecule. Preferably the biologic ally active compound is an adhesion polypeptide, preferably FN or vitronectin.

The biologically active compound preferably results in an improved contact between said material and a biological entity, such as a biological cell or a virus, or part thereof including a polypeptide, or a part thereof a nucleic acid moiety, a carbohydrate moiety, and a lipid moiety, including any combination thereof.

In one particularly preferred embodiment the material according to the invention further comprises a second determinant as defined herein. The second determinant comprises a biological entity, such as a biological cell or a virus, or part thereof, including a polypeptide, or a part thereof, a nucleic acid moiety, a carbohydrate moiety, and a lipid moiety, including any combination thereof.

The biological entity is preferably also selected from the group consisting of a polypeptide, an antibody, a polyclonal antibody, a monoclonal antibody, an immunogenic determinant, an antigenic determinant, a receptor, a receptor binding protein, an interleukine, a cytokine, a differentiation factor, a growth factor, and an antagonist to the receptor. The biological cell, or part thereof, is preferably a mammalian cell, including a human cell and an animal cell, a plant cell, a microbial cell, including a eukaryotic microbial cell including a yeast and a fungus, and a prokaryotic microbial cell including a bacteria.

The second determinant may also be a mammalian virus, including a human virus and an animal virus, a plant virus, a microbial virus, including a eukaryotic microbial virus, including a yeast virus and a fungal virus, and a prokaryotic microbial virus including a bacteriophage.

In one embodiment the substratum is porous and preferably a membrane. The flux of water through said material is preferably substantially unchanged as compared to the flux of water through said porous substratum. In another embodiment the substratum is non-porous and/or substantially non-penetrable to water.

There is also provided a material for use in a method of controlling cellular growth and/or cellular proliferation and/or cellular differentiation ex vivo, or a method of separating and/or isolating biological material ex vivo, or a method of producing a biohybrid organ ex vivo.

In another embodiment there is provided a material for use in a diagnostic method carried out on, the human or animal body, or for use in a method of therapy carried out on the human or animal body, or for use in a method of surgery carried out on the human or animal body.

There is also provided a material for use in a method of producing a biohybrid organ in vivo, and a material for use as a carrier for in vivo delivery of a medicament to a human or animal body in need of said medicament. In another embodiment there is provided a material for use in a method of controlling cellular growth and/or cellular proliferation and/or cellular differentiation in vivo, and a material for use in a method of separating and/or isolating biological material in vivo.

In another aspect there is provided a composition comprising the material according to the invention and a physiologically acceptable carrier. The invention also pertains to a pharmaceutical composition comprising the material according to the invention or the composition as defined herein and a pharmaceutically active ingredient and optionally a pharmaceutically active carrier.

The pharmaceutically active compound is preferably selected from the group consisting of enzymes, hormones, cytokines, colony stimulating factors, vaccine antigens, antibodies, clotting factors, regulatory proteins, transcription factors, receptors, structural proteins, angiogenesis factors, human growth hormone, Factor VIII, Factor IX, erythropoietin, insulin, alpha-1 antitrypsin, calcitonin, glucocerebrosidase, low density lipoprotein (LDL) receptor, IL-2 receptor, globin, immunoglobulin, catalytic antibodies, the interleukins, insulin-like growth factor 1 (IGF-1), parathyroid hormone (PTH), leptin, the interferons, the nerve growth factors, basic fibroblast growth factor (bFGF), transforming growth factor (TGF), transforming growth factor-beta (TGF-beta), acidic FGF (aFGF), epidermal growth factor (EGF), endothelial cell growth factor, platelet derived growth factor (PDGF), transforming growth factors, endothelial cell stimulating angiogenesis factor (ESAF), angiogenin, tissue plasminogen activator (t-PA), granulocyte colony stimulating factor (G-CSF), and granulocyte-macrophage colony stimulating factor (GM-CSF).

There is also provided the use of the material or the composition or the pharmaceutical composition according to the invention in a method of therapy carried out on the human or animal body, a method of surgery carried out on the human or animal body, or a diagnostic method carried out on the human or animal body.

In another embodiment there is provided the use of the material or the composition or the pharmaceutical composition in a method of producing a biohybrid organ in vivo, or as a carrier for in vivo delivery of a medicament to a human or animal body in need of said medicament

The invention also pertain to the use of the material or the composition or the pharmaceutical composition in a method of controlling cellular growth and/or cellular proliferation and/or cellular differentiation in vivo, or use of the material in a method of separating and/or isolating biological material in vivo, or use of the material in a method of controlling cellular growth and/or cellular proliferation and/or cellular differentiation ex vivo, or use of the material in a method of separating and/or isolating biological material ex vivo, or use of the material in a method of producing a biohybrid organ ex vivo, and the use of the material in the manufacture of an implantable organ or part thereof.

The material according to the invention may also be used as a carrier for a pharmaceutically active ingredient or a pharmaceutical composition.

There is also provided a method of controlling cellular growth and/or cellular proliferation and/or cellular differentiation ex vivo, said method comprising the steps of contacting a cell with the material or the composition or the pharmaceutical composition according to the invention, and incubating said cell and said material under conditions allowing said cell to grow and/or proliferate and/or differentiate.

The invention also pertains to a method of separating and/or isolating biological material ex vivo, said method comprising the steps of contacting said biological material to be separated and/or isolated with the material or the composition or the pharmaceutical composition according to the invention, and incubating said biological material and said material under conditions that allow separation and/or isolation.

There is also provided a method of producing a biohybrid organ ex vivo, said method comprising the steps of contacting biohybrid organ cells with the material or the composition or the pharmaceutical composition according to the invention, and incubating said biohybrid organ cells under conditions allowing the production of said biohybrid organ.

The invention also pertains to the following methods in particularly preferred embodiments:

Method of therapy carried out on the human or animal body, said method comprising the step of contacting said body with the material or the composition or the pharmaceutical composition according to the invention.

Method of surgery carried out on the human or animal body, said method comprising the step of contacting said body the material or the composition or the pharmaceutical composition according to the invention.

Method of diagnosis carried out on the human or animal body, said method comprising the steps of contacting said body with the material or the composition or the pharmaceutical composition according to the invention, and detecting a signal generated directly or indirectly by said material.

Method of controlling cellular growth and/or cellular proliferation and/or cellular differentiation in vivo, said method comprising the steps of contacting a cell with the material or the composition or the pharmaceutical composition according to the invention, and incubating said cell and said material under conditions allowing said cell to grow and/or proliferate and/or differentiate.

Method of separating and/or isolating biological material in vivo, said method comprising the steps of contacting said biological material to be separated and/or isolated with the material or the composition or the pharmaceutical composition according to the invention, and incubating said biological material and said material under conditions that allow separation and/or isolation.

Method of producing a biohybrid organ in vivo, said method comprising the steps of contacting biohybrid organ cells with the material or the composition or the pharmaceutical composition according to the invention, and incubating said biohybrid organ cells under conditions allowing the production of said biohybrid organ.

Method of in vivo delivery of a medicament to a human or animal body in need of said medicament, said method comprising the steps of contacting said body with the pharmaceutical composition according to the invention and incubating said body contacted by said pharmaceutical composition under conditions allowing delivery of said medicament.

Additionally preferred embodiments of the invention are illustrated herein below. U.S. Pat. No. 5,201,715 incorporated herein by reference relates to a target object having a characteristic ultrasonic signature for implantation beneath the skin. The object, when placed within an implanted injection port enables ultrasonic echographic discrimination of the target from surrounding tissues. The object enables one to locate the position of the object beneath the skin by non-invasive ultrasonic echograpy. The signature comprises reflections of ultrasonic waves from the object.

Accordingly, one embodiment of the present invention relates to a device for implantation beneath the skin capable of being located by non-invasive ultrasonic means at least when implanted. The device comprises a target comprising a biocampatible material according to the present invention comprising at least one and preferably a plurality of ultrasonically reflective surfaces, said at least one or a combination of said plurality of ultrasonically reflective surfaces providing a characteristic ultrasonic echographic signature. The biocompatible material preferably has an acoustical velocity which is different from the acoustical velocity of human tissue, and the object optionally further comprises a laminate structure consisting of substantially planar layers of bonded together biocompatible materials according to the present invention. The object preferably comprises a unitary structure.

U.S. Pat. No. 5,976,780 incorporated herein by reference relates to a macroencapsulation device for somatic cells. Accordingly, the present invention in one embodiment relates to a transplantation or implantation device comprising
i) a hollow fiber comprising a material according to the present invention and having ends and a fiber wall with a porosity which selectively allows nutritional, gaseous, and metabolic substances to pass therethrough and which only allows passage of substances having a molecular weight less than about 30,000 Daltons, and
ii) a mixture of viable cells, preferably somatic, mammalian cells, and alginate gel suspended within said fiber.

Also provided is a device wherein said wall is devoid of macrovoids and has a porosity which prevents donor antigens and cytokines from passing through said wall. The device preferably comprises a fiber comprising a material according to the present invention capable of inhibiting complement activation. The somatic cells are preferably selected from the group consisting of neural, endocrine and hepatic cells, and said cells are preferably free from passenger leukocytes.

Also provided is a transplantation or implantation device comprising
i) a hollow fiber comprising a material according to the present invention and having ends and a fiber wall with a porosity which selectively allows nutritional, gaseous, and metabolic substances to pass therethrough and
ii) a mixture of viable cells, preferably viable, somatic, mammalian cells, and alginate gel suspended within said fiber.

The alginate optionally comprises ultrapurified alginate which is substantially free of divalent metal toxins and comprises. (i) an endotoxin content of preferably less than 750 EU/g, (ii) a protein content of preferably less than 0.2%, and (iii) a G monomer, dimer and trimer content of preferably greater than 60%.

U.S. Pat. No. 4,624,669 incorporated herein by reference relates to a corneal inlay for implant within the cornea and of a material such as polysulfone, wherein the inlay comprises a plurality of pores facilitating the passage of nutrients and fluids from the bottom surface layer of the cornea to the top surface layer of the cornea. Accordingly, one embodiment of the present invention pertains to a corneal inlay comprising:
i) an optic lens comprising a material according to the present invention for implantation within the cornea; and,
ii) a plurality of holes having a diameter of from 0.001 mm to 0.1 mm, said holes extending from a bottom surface to a top surface so as to allow for passage of nutrients through the cornea.

Also provided is a corneal inlay comprising:
i) an optic lens comprising a material according to the present invention for implant within the cornea; and,
ii) a plurality of slits, having a maximum width of from 0.01 mm to 0.05 mm, and a maximum length of from 0.05 mm to 1.0 mm, said slits extending from a bottom surface to a top surface so as to allow for passage through the cornea.

U.S. Pat. No. 5,213,721 incorporated herein by reference relates to a porous device comprising a plurality of holes arranged in a predetermined, geometrical configuration. The holes are derived by means of a procedure of repetitive drawing. Prior to the first drawing operation, each of the holes is filled with a material which is: soluble to a certain chemical, yet drawable along with the base material. Dependent upon the extent of drawing, a porous device is provided which includes holes of a significantly reduced cross-sectional area.

Accordingly, there is provided a device comprising a material according to the present invention for use as either a scaffold, a contact lens, an intracorneal inlay, an intraocular lens, a medical filter, or a similar structure with small holes. Accordingly, there is provided a scaffold or an optic device such as a contact lens comprising a material according to the present invention.

U.S. Pat. No. 5,965,125 incorporated herein by reference relates to an implantable device having a body of matrix material made up of insoluble collagen fibrils, and disposed therewithin i) a plurality of vertebrate cells; and ii) a plurality of microspheres including microspheres consisting primarily of polysulfone.

Accordingly, the present invention in one embodiment relates to a composition comprising a body of matrix material, preferably a matrix material comprising insoluble collagen fibrils, and embedded within the body of said matrix material
i) a plurality of cultured cells, preferably vertebrate cells, even more preferably genetically engineered vertebrate cells, wherein said cells are capable of expressing a medically useful biologically active compound including a polypeptide; and
ii) a plurality of microspheres; wherein at least part of said microspheres comprises a material according to the present invention.

The cultured vertebrate cells are preferably selected from the group consisting of adipocytes, astrocytes, cardiac muscle cells, chondrocytes, endothelial cells, epithelial cells, fibroblasts, gangliocytes, glandular cells, glial cells, hematopoietic cells, hepatocytes, keratinocytes, myoblasts, neural cells, osteoblasts, pancreatic beta cells, renal cells, smooth muscle cells, striated muscle cells, and precursors of any of the above.

It is preferred that the cultured vertebrate cells are transfected cells, preferably transfected human cells comprising exogenous DNA encoding a medically useful biologically active compound including a polypeptide. The cultured vertebrate cells are preferably transfected cells containing exogenous DNA which includes a regulatory sequence that activates expression of a gene encoding said medically useful biologically active compound, preferably a polypeptide, wherein said gene is endogenous to said vertebrate cells both prior to and after they are transfected.

The biologically active compound, preferably a polypeptide, is preferably selected from the group consisting of enzymes, hormones, cytokines, colony stimulating factors, vaccine antigens, antibodies, clotting factors, regulatory proteins, transcription factors, receptors, structural proteins, angiogenesis factors, human growth hormone, Factor VIII, Factor IX, erythropoietin insulin alpha-1 antitrypsin, calcitonin, glucocerebrosidase, low density lipoprotein (LDL) receptor, IL2 receptor, globin, immunoglobulin, catalytic antibodies, the interleukins, insulin-like growth factor 1. (IGF-1), parathyroid hormone (PTH), leptin, the interferons, the nerve growth factors, basic fibroblast growth factor (bFGF), transforming growth factor (TGF), transforming growth factor-beta (TGF-beta), acidic FGF (aFGF), epidermal growth factor (EGF), endothelial cell growth factor, platelet derived growth factor (PDGF), transforming growth factors, endothelial cell stimulating angiogenesis factor (ESAF), angiogenin, tissue plasminogen activator (t-PA), granulocyte colony stimulating factor (G-CSF), and granulocyte-macrophage colony stimulating factor (GM-CSF).

In a preferred embodiment, the biologically active compound, preferably in the form of a medically useful polypeptide, is administered to a patient by shunting a portion of the patient's blood so that the polypeptide secreted by the cells in the hybrid matrix mixes with the blood. Generally, any suitable method known to those of skill in the art can be used or adapted to accommodate the matrix of the invention. For example, blood shunted into a device which contains a perm-selective membrane surrounding a matrix comprising a material according to the present invention will result in the delivery of a therapeutic product of the matrix to the blood. A device similar to an artificial pancreas (Sullivan et al., Science 252:718-721, 1991) may be used for this purpose.

In another preferred embodiment, a hybrid matrix comprising a material according to the present invention is a means for producing a polypeptide in vitro. The method includes the steps of placing the hybrid matrix comprising a material according to the present invention under conditions whereby the cells in the matrix express and secrete a polypeptide of interest; contacting the matrix with a predetermined liquid such that the cells secrete the polypeptide into said liquid; and obtaining the polypeptide from the liquid, e.g., by standard purification techniques appropriate for the given polypeptide.

In one preferred embodiment, the matrix comprising a material according to the present invention is anchored to a surface and is bathed by the liquid; alternatively, the matrix floats freely in the liquid. Cells embedded in the hybrid matrix preferably function at a high level in a relatively confined space. Furthermore, the first step in purification of e.g. an expressed polypeptide (removal of the cells from the medium) is considerably more efficient with the matrices according to the present invention than with most standard methods of cell culture.

U.S. Pat. No. 5,676,924 incorporated herein by reference relates to a method of determining the effectiveness of a cancer treatment by sealing tumor cells in segments of semipermeable membrane hollow fibers, implanting the sealed fiber segments in a mammal, treating the mammal with a cancer treatment, and evaluating the effect of the cancer treatment on the cells in the hollow fiber segments.

Accordingly, the present invention in one embodiment relates to a method of determining the effectiveness of a cancer treatment, said method comprising the steps of,
i) providing elongated segments of semipermeable membrane hollow fibers comprising a material according to the present invention and having a pore size effective to permit passage of nutrients, wherein said pore size excludes components of a the immune system of a mammal, wherein said components are capable of inducing tissue rejection,
ii) placing by means of inserting said tumor cells into said hollow fibers, and sealing the ends of said hollow fibers,
iii) implanting said sealed hollow fibers intraperitoneally or subcutaneously into a non-human mammal,
iv) administering the cancer treatment and
v) monitoring the effectiveness of said treatment on the cells in said implanted hollow fibers.

The mammal is preferably immunocompetent, such as a rat, and the tumor cells are preferably leukemic cells, preferably autologous leukemic cells or tumor cells obtained from a tumor of a patient. The tumor cells by be in suspension or in the form of a tissue explant.

U.S. Pat. No. 5,830,708 incorporated herein by reference relates to methods for producing naturally secreted human extracellular matrix material and compositions containing this extracellular matrix material. The method includes culturing extracellular matrix-secreting human cells on a biocompatible, three-dimensional framework in vitro.

Accordingly, the present invention in one embodiment relates to a method for the production of human, naturally secreted extracellular matrix material, said method comprising the steps of:
i) providing a) a living tissue, optionally tissue prepared in vitro, preferably by culturing living tissue comprising human stromal cells such as fibroblasts, and b) connective tissue proteins naturally secreted by the living tissue, said connective tissue being attached to and substantially enveloping a material according to the present invention;
ii) killing the cells in the living tissue; and
iii) removing the killed cells and any cellular contents from the material according to the present invention, and
iv) collecting the extracellular matrix material deposited on the framework.

The method optionally further comprises the step of processing the collected extracellular matrix material by homogenizing, cross-linking, or suspending the extracellular matrix material in a physiological acceptable carrier.

The stromal cells, preferably fibroblasts, of the living stromal tissue are cells found in loose connective tissue or bone marrow, and preferably endothelial cells, pericytes, macrophages, monocytes, leukocytes, plasma cells, mast cells or adipocytes.

In one embodiment of this aspect of the invention there is provided an injectable material for soft tissue augmentation and related methods for use and manufacture of such materials, which overcome the shortcomings of state of the art bovine injectable collagen and similar injectable materials. The injectable materials according to the present invention comprise naturally secreted extracellular matrix preparations as well as preparations derived from naturally secreted extracellular matrix. These preparations are biocompatible, biodegradable and are capable of promoting connective tissue deposition, angiogenesis, reepithilialization and fibroplasia, which is useful in the repair of skin and other tissue defects. These extracellular matrix preparations may be used to repair tissue defects by injection at the site of the defect.

In another embodiment of the present invention, the preparations can be used in highly improved systems for in vitro tissue culture. Naturally secreted extracellular matrix coated three-dimensional frameworks comprising a material according to the present invention can be used to culture cells which require attachment to a support in order to grow, but do not attach to conventional tissue culture vessels. In addition to culturing cells on a coated framework, the extracellular matrix secreted by the cells onto the framework can be collected and used to coat vessels for use in tissue culture. The extracellular matrix, acting as a base substrate, may allow cells normally unable to attach to conventional tissue culture dish base substrates to attach and subsequently grow.

Yet another embodiment of the present invention is directed to a novel method for determining the ability for cellular taxis of a particular cell. The method involves inoculating one end of a native extracellular matrix coated three-dimensional framework comprising a material according to the present invention with the cell type in question, and over time measure the distance traversed across the framework by the cell. Because the extracellular matrix is secreted naturally by the cells onto the framework, it is an excellent in vitro equivalent of extracellular matrix found in the body. Such an assay, for example, may inform whether isolated tumor cells are metastatic or whether certain immune cells can migrate across or even chemotact across the framework, thus, indicating that the cell has such cellular taxis ability.

In another aspect of the present invention there is provided a method for producing the material according to the invention, said method comprising the steps of providing a substratum having a second contact angle, and contacting said substratum with a composition comprising a plurality of macromolecules. The method preferably pertains to the production of a material as described herein above. The substratum preferably comprises a hydrophobic polymer and said substratum may be pretreated prior to being contacted by said macromolecule. The pretreatment is effective in increasing the wettability of said substratum.

The macromolecule according to the method comprises a hydrophilic polymer, preferably a latently reactive polymer. The macromolecule preferably has a MW of more than 400 Da The macromolecule comprises a conjugate comprising a likable head group, a linker group, a polymer chain, and a functional end group. The head group is preferably a photo-reactive aryl azide head group.

The macromolecule may optionally comprise a modifying agent, preferably a modifying agent capable of contacting said substratum and forming a self assembled monolayer.

According to the method for producing the material according to the invention, said method may comprising the further step of contacting said material with a first determinant comprising a biologically active compound. The biologically active compound is preferably a polypeptide, an antibody, a polyclonal antibody, a monoclonal antibody, an immunogenic determinant, an antigenic determinant, a receptor, a receptor binding protein, an interleukine, a cytokine, a cellular differentiation factor, a cellular growth factor, or an antagonist to a receptor. The biologically active compound may be membrane associated and/or an extracellular matrix polypeptide natively produced by a microbial cell, a plant cell or a mammalian cell.

According to the method of the invention, a further step of contacting said material with a second determinant comprising a biological entity may also be included. The biological entity comprises a cell or a virus, or a part thereof, and said cell, or part thereof, is preferably selected from the group consisting of a mammalian cell, including a human cell and an animal cell, a plant cell, a microbial cell, including a eukaryotic microbial cell, including a yeast and a fungus, and a prokaryotic microbial cell including a bacteria. When being a virus, or part thereof said virus is preferably selected from a mammalian virus, including a human virus and an animal virus, a plant virus, a microbial virus, including a eukaryotic microbial virus, including a yeast virus and a fungal virus, and a prokaryotic microbial virus including a bacteriophage. Accordingly, the biological entity as defined herein preferably comprises a polypeptide, or a part thereof a nucleic acid moiety, a carbohydrate moiety, and a lipid moiety, including any combination thereof. The biological entity may also comprise an antibody, a polyclonal antibody, a monoclonal antibody, an immunogenic determinant, an antigenic determinant, a receptor, a receptor binding protein, an interleukine, a cytokine, a differentiation factor, a growth factor, or an antagonist to the receptor.

The method of producing a material according to the invention relates in one preferred embodiment to a modification of a method described in U.S. Pat. No. 5,741,551 (to Guire). Accordingly, the novel biomaterial surface layer is in one preferred embodiment generated by a two-step process using e.g. macromolecular amphiphiles with latent (photo) reactivity. Consequently, in a first step, amphiphilic macromolecules are allowed to adsorb to a suitable polymer substratum. The latent-reactive head-group will bring the amphiphils into reactive contact with the surface of the substratum. The hydrophilic main-body of the amphiphilic macromoledules exhibits a pronounced excluded volume leading to a lateral pattern of uniformly “self-assembled”, adsorbed amphiphilic macromolecules. As described above, layer density and pattern depend on e.g. the amphiphilic character of the macromolecule such as e.g. chain length and/or degree of branching, the polymer substratum, as well as the solution conditions (e.g. concentration, solvent, salt, temperature). As a consequence, the interface properties will be adjustable by altering the molecular characteristics of both the polymer substratum and the macromolecule. Similar or at least substantially similar monolayer structures are attainable on even quite different substrata by adjusting e.g. macromolecular properties or solution conditions. Amphiphil adsorption can readily be monitored by known surface physico-chemical methods such as e.g. ellipsometry or contact angle (CA) measurements.

In a second step, excess of macromolecules is removed and the latently reactive head-groups are activated. The activation results in the formation of a covalent bond formation between the macromolecule and the surface of the polymer substratum. Activation is preferably achieved by using electromagnetic radiation in the UV or Vis light range.

In a preferred embodiment, the method of producing a material according to the present invention is practiced with a macromolecule comprising a hydrophilic polymer, the hydrophilic polymer preferably being poly(ethylene glycol). The macromolecule preferably has a MW of more than 400 Da The macromolecule further comprises a conjugate comprising a linkable head group, a linker group, a polymer chain, and a functional end group. The head group preferably is a photo-reactive aryl azide head group. In this preferred embodiment no irradiation is applied to the substratum, being contacted with said macromolecule, which could activate the latently reactive head group forming a covalent bond between the substratum and said macromolecule.

Without being bound by theory it is believed, that the macromolecules contacting said substratum are anchored/immobilized to the underlying substratum by hydrophobic interactions and/or entanglement of the headrgroup/guiding group and the hydrophobic substratum.

In one preferred embodiment, the method according to the present invention is practiced on a substratum that has not been pretreated. Substrata such as solid surfaces may be pre-washed to remove surface contamination and may be modified as desired to affect solvophilic characteristics without adding functional groups that are involved in covalent bond formation with e.g. latent-reactive groups. For example, polystyrene surfaces may be washed and then exposed to hydroxyl ions in known water vapour plasma contact procedures so as to add hydroxyl groups to the substratum surface solely for the purpose of rendering the surface more readily wetted by aqueous solutions, the hydroxyl groups not being involved in subsequent covalent bond formation with the surface upon latent reactive group activation. Avoidance of pretreatment steps, defined in the definitions, leads not only to important processing economies but also avoids technical problems associated with the attachment of bond-forming reactive groups to surfaces at uniform loading densities.

Diagnosis

In one embodiment of the present invention the materials comprising a substratum as described herein above are used in a diagnostic method.

Such a diagnostic method may be carried out on a human or animal body. For example the diagnosis may be performed in vivo on a human or animal body or the diagnosis may be performed on samples from a human or animal body. Such sample may be used directly or they may be processed prior to diagnosis.

Diagnosis may be performed by any suitable assay known to the person skilled in the art. Preferably, the diagnosis comprises the use of a solid support, which comprises, essentially consists of or consists of one or more, materials according to the present invention.

In preferred embodiments diagnosis comprises detection of one or more markers indicative of the clinical condition, which is desirable to diagnose. In such an embodiements, assays based on a specific recognition of such marker(s) and/or antigenic determinants associated with said markers are preferred, such as qualitative and/or quantitative assays involving the use of immunoreactive species, i.e. antigens, haptens and antibodies or fragments thereof:

The term antigenic determinant according to the present invention encompasses any molecule or parts thereof, which may be recognised by an immunoreactive species, for example an antigenic determinant may be an antigen or an epitope.

The present invention may in one embodiment employ standard immunohistochemical or cytochemical detection procedures, or suitable modifications thereof, for the detection of a marker indicative of a given condition and/or an antigenic determinant assocaited therewith. Accordingly, the invention may employ any assay resulting in the recognition of an antigenic determinant mediated by an immunochemical reaction of the antigenic determinant with a specific so-called primary antibody capable of reacting exclusively with the target antigenic determinant for example in the form of a marker.

The primary antibody is preferably labelled with an appropriate label capable of generating —directly or indirectly—a detectable signal. The label is preferably an enzyme, a radioactive isotope, a fluorescent group, a dye, a chemiluminescent molecule and a heavy metal such as gold.

In another embodiment, the invention employ the detection of the primary antibody by immunochemical reaction with specific so-called secondary antibodies capable of reacting specifically with the primary antibodies. In this case the secondary antibodies are preferably labelled with an appropriate label such as an enzyme, a radioactive isotope, a fluorescent group, a dye, a chemiluminescent molecule or a heavy metal such as gold.

In yet another embodiment, the present invention employs a so-called linker antibody as a means of detection of the marker. This embodiment exploits that the immunochemical reaction between the target antigenic determinant in the form of the marker and the primary antibody is mediated by another immunochemical reaction involving the specific linker antibody capable of reacting simultaneously with both the primary antibody as well as another antibody to which enzymes have been attached via an immunochemical reaction, or via covalent coupling and the like.

In yet another embodiment according to the present invention, the immunochemical reaction between the target antigenic determinant in the form of the marker and the primary antibody, or alternatively, between the primary antibody and the secondary antibody, is detected by means of a binding of pairs of complementary molecules other than antigens and antibodies. A complementary pair such as e.g. biotin and streptavidin is preferred. In this embodiment, one member of the complementary pair is attached to the primary or secondary antibody, and the other member of the complementory pair is contacted by any suitable label such as e.g. an enzymes, aradioactiveisotope, a fluorescent group, a dye or a heavy metal such as gold.

A sample is preferably brought into contact with a carrier and optionally treated with various chemicals to facilitate the subsequent immunochemical reactions. The sample contacting the carrier is referred to as a specimen. The sample in one preferred embodiment is then subjected to treatment with a labelled or non-labelled primary antibody, as appropriate, whereupon the antibody becomes immunochemically bound to the marker comprised in the sample. After removal of excess antibody by suitable washing of the specimen comprising the sample, the antibody bound to the marker is detected by reaction with appropriate reagents, depending on the choice of detection system.

After removing excess labelled reagent from the chosen detection system, the specimen comprising the marker to be detected and optionally also quantified is preferably subjected to at least one of the detection reactions described below. The choice of detection reaction is influenced by the marker in question as well as by the label it is decided to use.

When an enzyme label is used, the specimen is treated with a substrate, preferably a colour developing reagent. The enzyme reacts with the substrate, and this in turn leads to the formation of a coloured, insoluble deposit at and around the location of the enzyme. The formation of a colour reaction is a positive indication of the presence of the marker in the specimen.

When a heavy metal label such as gold is used, the specimen is preferably treated with a so-called enhancer in the form of a reagent containing e.g. silver or a similar contrasting indicator. Silver metal is preferably precipitated as a black deposit at and around the location of the gold.

When a fluorescent label is used, a developing reagent is normally not needed.

After at least one washing step, some of the constituents of the specimen are preferably coloured by reaction with a suitable dye resulting in a desirable contrast to the colour provided by the label in question. After a final washing step, the specimen is preferably coated with a transparent reagent to ensure a permanent record for the examination.

Detection of the label in question preferably indicate both the localization and the amount of the target antigenic determinant in the form of the marker, indicative of a condition. The detection may be performed by visual inspection, by light microscopic examination in the case of enzyme labels, by light or electron microscopic examination in the case of heavy metal labels, by fluorescence microscopic examination, using irradiated light of a suitable wavelength, in the case of fluorescent labels, and by autoradiography in the case of an isotope label.

Enzyme-Linked Immuno-Sorbent Assays (ELISA) in which an antigen, hapten or antibody is detected by means of an enzyme which is linked such as covalently coupled or conjugated either—when an antigen or hapten is to be determined—to an antibody which is specific for the antigen or hapten in question, or—when an antibody is to be determined—to an antibody which is specific for the antibody in question—may be used for detecting the markers indicative of a given condition.

In one preferred embodiment, the marker to be detected is bound or immobilized by immunochemically contacting the marker with a so-called “catching” antibody attached by e.g. non-covalent adsorption to the surface of an appropriate material. Examples of such materials are polymers such as e.g. nitrocellulose or polystyrene, optionally in the form of a stick, a test strip, a bead or a microtiter tray. A suitable enzyme-linked specific antibody is allowed to bind to the immobilized marker to be detected. The amount of bound specific antibody, i.e. a parameter that is correlatable to the immobilized marker, is determined by adding a substance capable of acting as a substrate for the linked enzyme. Enzymatic catalysis of the substrate results in the development of a detectable signal such as e.g. a characteristic colour or a source of electromagnetic radiation. The intensity of the emitted radiation can be measured e.g. by spectrophotometry, by colorimetry, or by comparimetry. The determined intensity of the emitted radiation is correlatable—and preferably proportional—to the quantity of the marker to be determined. Examples of preferred enzymes for use in assays of this type are e.g. peroxidases such as horseradish peroxidase, alkaline phosphatase, glucose oxidases, galactosidases and ureases.

It is one objective of the present invention, the assays involve immobilisation of the marker(s) on an solid support using a targeting species, preferably an antibody. The solid support used in the present invention may be employed in a variety of forms or structures. The solid support has a location where the targeting species can bind or associate, and the formation of such an solid support with said targeting species, preferably an antibody, enables contacting a sample and other materials used in the method of the invention.

Preferably, the solid support is formed in a way which enables simple manipulation for easy contact with the sample and other reagents. For example, the samples and other reagents can be drawn in and ejected from a syringe, caused to flow through a tube, or deposited in a container such as a test tube shaped container.

The solid support is composed of any material onto which the desired targeting species, preferably an antibody, can be effectively bound. For covalent binding with antibody protein, the solid support material can be chosen to contain a functional carboxyl surface, with use of a water-soluble carbodiimide as a conjugation reagent. A preferred material is acrylic resin, which has a carboxylated surface that enables binding the desired targeting species, preferably an antibody by conjugation. For materials with amino surface groups, reactive carboxyl intermediates can be prepared by reacting with succinic anhydride. A variety of inorganic supports, typically glass, can also be prepared for covalent coupling with targeting species, preferably an antibody. Reference is made, for example, to “Enzymology, A Series of Textbooks and Monographs,” Vol. 1, Chapter 1, 1975, the disclosure of which is incorporated herein by reference.

In one embodiment, the present method employs a direct binding assay instead of a competitive binding assay where a dynamic equilibrium necessitates lengthy incubation. The disclosed method can, of course, be employed in a competitive protein binding assay as well. The roles of the immune analytes antibody and antigen can also be interchanged, still making use of the immobilized solid support for the signal amplification. Binding of antibody or various antigen molecules to the solid support matter is well known, in passive adsorption as well as in covalent coupling.

The method of the invention can also be designed to assay several markers in a single procedure where each marker is represented by a particular pair of corresponding binding partners including antibodies, antigens.

Detection of different types of markers can be done in accordance with the invention by conjugating a plurality of different targeting species, preferably antibodies, capable of forming complexes with different blood coagulation markers, to the solid support and to the reporter species. The detection of bound material as described above following the assay indicates that one or more of the different blood coagulation markers are present in the specimen, and this assay, if positive, can be followed by assays for individual blood coagulation markers selcted from the ones which were tested for simultaneously. Immunochemical assays of a type analogous to ELISA but employing other means of detection are also suitable for detecting the marker according to the present invention. Such assays are typically based on the use of specific antibodies to which fluorescent or luminescent marker molecules are covalently attached. So-called. “time-resolved fluorescence” assays are particularly preferred and typically employ an europium ion label or an europium chelator, even though certain other lanthanide species or lanthanide chelators may also be employed. In contrast to many traditional fluorescent marker species the fluorescence lifetime of lanthanide chelates is generally in the range of 100-1000 microseconds. In comparison, fluorescein has a fluorescence lifetime of only about 100 nanoseconds or less. By making use of a pulsed light source and a time-gated fluorometer, the fluorescence of lanthanide chelate compounds can be measured in a time-window of about 200-600 microseconds after each excitation. A main advantage of this technique is the reduction of background signals which may arise from more short-lived fluorescence of other substances present in the analysis sample or in the measurement system.

It is another object of the present invention to detect markers in a sample by means of miniaturized, integrated microfluid devices and systems incorporating such devices.

Additional assays employing immunochemical detection techniques capable of being exploited in the present invention belong to the group of “immunoblotting” procedures, such as e.g. “dot blot” and “western blot” procedures.

In one embodiment the method of diagnosis involved the use of sensor laminates, multi-sectioned fluid delivery devices or the like, such as the sensor laminates or the multisectioned fluid delivery devices, which are described in the international patent application WO 98/25141, which is hereby incorporated by reference in its entirety. Preferably, such sensor laminates, multi-sectioned fluid delivery devices or the like comprise or essentially consist of materials-as described by the present invention.

Such sensor laminates comprises a ligand, which can associate with a marker indicative of a given condition, wherein said ligand is bound to a polymeric material, which has been treated to initiate formation of free radicals. Such treatment may for example be irradiation by an electron beam or by sonochemical techniques. The polymeric material may for example be selected from the group consisting of polystyrene polysiloxane, polystyrene-butadiene co-polymers, polyethylene, polypropylene, ethylene vinyl acetate, polyvinylchloride, tetrafluoroethylene, polycarbonate and polysulfone which have a fiber size which renders them nonporous.

Preferably, the ligand is comprised within a reactive substrate layer, which may be comprised of a selected ligand for the target molecule interspersed widely throughout the layer and bound to a polymeric material treated to enhance binding of the ligand to the polymeric material. Examples of polymeric materials which can be used in the reactive substrate layer also include, but are not limited to, polystyrene, polysiloxane, polystyrene-butadiene co-polymers, polyethylene, polypropylene, ethylene vinyl acetate, polyvinylchloride, tetrafluoroethylene, polycarbonate and polysulfone. Furthermore, the sensor laminates may comprise a top sample activation layer comprised of a soluble material such as 3% citric acid in polyvinyl pyrrolidone which promotes the production of ample quantities of sample and permits diffusion of target molecules in the sample placed upon this layer into the reactive substrate layer beneath. The sample diffuses into the reactive substrate layer, wherein target molecules in the sample bind to ligand. Bound target molecules are then detected by contacting the reactive substrate layer 3 with standard detection reagents used routinely in ELISAs for detection of a bound target molecule. For example, in one embodiment, a detection reagent may comprise a second ligand for the target molecule which is detectably labeled. Examples of detectable labels include fluorometric agents such as fluorescein isothiocyanate or calorimetric agents such as horse radish peroxidase. Additional reagents required for detection of such labels are well known in the art.

Containers

In a further embodiment of the present invention, the material disclosed herein may be used for containers. For example, a container may comprise, essentially consist of or consist of the materials disclosed by the present invention.

The term container is used herein to cover any receptacle, such as a test tube, Microtiter plate, dish, carton, can, or jar, in which material may be stored, held or carried. A container may be sealable or not sealable, may comprise a lid or may be open to the surrounding environment. A container according to the present invention may have any desirable shape.

The container may be prepared from a number of different materials, including any of polymers as described herein above.

In a preferred embodiment of the present invention, the container may be useful for storage of biologically active substance for any desirable amount of time.

A biologically active substance according to the present invention may in one embodiment comprise or consist of a peptide, such as a polypeptide or an oligopeptide. Examples of polypeptides and/or oligopeptides to be stored in a container according to the present invention are antibodies and fragments thereof, antigens for example for use in vaccines, hormones, enzymes, signalling molecules, polypeptides and/or oligopeptides, which can act as inhibitors or activators of other proteins, cytokines, vitamins, transcription factors and the like.

In one preferred embodiment the polypeptides and/or oligopeptides are usefull as medicament. Hence, the containers according to the present invention, may be useful for storing a biologically active substance, such as a medicament. Preferably a biologically active substance comprises one or more polypeptides and/or oligopeptides. Example of biologically active substances according to the present invention includes but are not limited to vitamins; substances used for the treatment, prevention, diagnosis, cure or mitigation of disease or illness; or substances which affect the structure or function of the body; or pro-drugs, which become biologically active or more active after they have been placed in a predetermined physiological environment.

Non-limiting examples of useful biologically active substances include the following expanded therapeutic categories: anabolic agents, antacids, anti-asthmatic agents, anti-cholesterolemic and anti-lipid agents, anti-coagulants, anti-convulsants, anti-diarrheals, anti-emetics, anti-infective agents, anti-inflammatory agents, anti-manic agents, anti-nauseants, anti-neoplastic agents, anti-obesity agents, anti-pyretic and analgesic agents, anti-spasmodic agents, anti-thrombotic agents, anti-uricemic agents, anti-anginal agents, antihistamines, anti-tussives, appetite suppressants, biologicals, cerebral dilators, coronary dilators, decongestants, diuretics, diagnostic agents, erythropoietic agents, expectorants, gastrointestinal sedatives hyperglycemic agents, hypnotics, hypoglycemic agents, ion exchange resins, laxatives, mineral supplements, mucolytic agents, neuromuscular drugs, peripheral vasodilators, psychotropics, sedatives, stimulants, thyroid and anti-thyroid agents, uterine relaxants, vitamins, antigenic materials, analgetics and prodrugs.

Specific examples of useful biologically active substances from the above categories include: (a) anti-neoplastics such as androgen inhibitors, antimetabolites, cytotoxic agents, immunomodulators; (b) anti-tussives such as dextromethorphan, dextromethorphan hydrobromide, noscapine, carbetapentane citrate, and chlophedianol hydrochloride; (c) antihistamines such as chlorpheniramine maleate, phenindamine tartrate, zyrilamine maleate, doxylamine succinate, and phenyltcloxamine citrate; (d) decongestants such as phenylephrine hydrochloride, chenylpropanolamine hydrochloride, pseudoephedrine hydrochloride, and ephedrine; (e) various alkaloids such as codeine phosphate, codeine sulfate and morphine—(f) mineral supplements such as potassium chloride, zinc chloride, calcium carbonates, magnesium oxide, and other alkali metal and alkaline earth metal salts; (g) ion exchange resins such as cholestryramine, (h) anti-arrhythmics such as N-acetylprocainamide; (i) antipyretics and analgesics such as acetaminophen, aspirin and ibuprofen; (j) appetite suppressants such as phenyl-propanolamine hydrochloride or caffeine; k) expectorants such as guaifenesin; (l) antacids such as aluminum hydroxide and magnesium hydroxide; (m) biologicals such as peptides, polypeptides, proteins and amino acids, hormones, interferons or cytokines and other bioactive peptidic compounds, such as hGH, tPA, calcitonin, ANF, EPO and insulin; (n) anti-infective agents such as anti-fungals, anti-virals, antiseptics and antibiotics; and (O) antigenic materials, particularly those useful in vaccine applications.

To further illustrate, antimetabolites which can be formulated in the subject polymers include, but are not limited to, methotrexate, 5-fluorouracil, cytosine arabinoside (ara-C), 5-azacytidine, 6-mercaptopurine, 6-thioguanine, and fludarabine phosphate. Antitumor antibiotics may include but are not limited to doxorubicin, daunorubicin, dactinomycin, bleomycin, mitomycin C, plicamycin, idarubicin, and mitoxantrone. Vinca alkaloids and epipodophyllotoxins may include, but are not limited to vincristine, vinblastine, vindesine, etoposide, and teniposide.

Hormonal therapeutics can also be included in the polymeric matrices, such as corticosteriods (cortisone acetate, hydrocortisone, prednisone, prednisone, methyl prednisolone and dexamethasone), estrogens, (diethylstibesterol, estradiol, esterified estrogens, conjugated estrogen, chlorotiasnene), progestins (medroxyprogesterone acetate, hydroxy progesterone caproate, megestrol acetate), antiestrogens (tamoxifen), aromastase inhibitors (aminoglutethimide), androgens (testosterone propionate, methyltestosterone, fluoxymesterone, testolactone), antiandrogens (flutamide), LHRH analogues (leuprolide acetate), and endocrines for prostate cancer ketoconazole).

Other compounds which can be disposed in the contianer of the present invention include those classified as e.g. investigational drugs, and can include, but are not limited to alkylating agents such as Nimustine AZQ, BZQ, cyclodisone, DADAG, CB10-227, CY233, DABIS maleate, EDMN, Fotemustine, Hepsulfam, Hexamethylmelamine, Mafosamide, MDMS, PCNU, Spiromustine, TA-077, TCNU and Temozolomide; antimetabolites, such as acivicin, Azacytidine, 5-aza-deoxycytidine, A-TDA, Benzylidene glucose, Carbetimer, CB3717, Deazaguine mesylate, DODOX, Doxifluridine, DUP-785, 10-EDAM, Fazarabine, Fludarabine, MZPES, MMPR, PALA, PLAC, TCAR, TMQ, TNC-P and Piritrexim; antitumor antibodies, such as AMPAS, BWA770U, BWA773U, BWA502U, Amonafide, m-AMSA, CI-921, Datelliptium, Mitonafide, Piroxantrone, Aclarubicin, Cytorhodin, Epirubicin, esorubicin, Idarubicin, Iodo-doxorubicin, Marcellomycin, Menaril, Morpholino anthracyclines, Pirarubicin, and SM-5887; microtubule spindle inhibitors, such as Amphethinile, Navelbine, and Taxol; the alkyl-lysophospholipids, such as BM41-440, ET-18-OCH3; and Hexacyclophosphocholine; metallic compounds, such as Gallium Nitrate, CL286558, CL287110, Cycloplatam, DWA2114R, NK121, Iproplatin, Oxaliplatin, Spiroplatin, Spirogermanium, and Titanium compounds; and novel compounds such as, for example, Aphidoicolin glycinate, Ambazone, BSO, Caracemide, DSG, Didemnin, B, DMFO, Elsamicin, Espertatrucin, Flavone acetic acid, HMBA, HHT ICRF-187, Iododeoxyuridine, Ipomeanol, Liblomycin, Lonidamine, LY186641, MAP, MTQ, Merabarone SK&F104864, Suramin, Tallysomycin, Teniposide, THU and WR2721; and Toremifene, Trilosane, and zindoxifene.

Antitumor drugs that are radiation enhancers can also be atored in the container. Examples of such drugs include, for example, the chemotherapeutic agents 5′fluorouracil, mitomycin, cisplatin and its derivatives, taxol, bleomycins, daunomycins, and methamycins.

The invention may, additionally, be used for the treatment of infections. For such an application, antibiotics, either water soluble or water insoluble, may be stored in the containers. Antibiotics are well known to those of skill in the art, and include, for example, penicillins, cephalosporins, tetracyclines, ampicillin, aureothicin, bacitracin, chloramphenicol, cycloserine, erythromycin, gentamicin, gramacidins, kanamycins, neomycins, streptomycins, tobramycin, and vancomycin

Interferons, interleukins, tumor necrosis factor, and other protein biological response modifiers may furthermore be stored in the containers according to the present invention.

In one embodiment, the biologically active substance is selected from the group consisting of polysaccharides, growth factors, hormones, anti-angiogenesis factors; interferons or cytokines, and pro-drugs. In a particularly preferred embodiment, the biologically active substance is a therapeutic drug or pro-drug, most preferably a drug selected from the group consisting of chemotherapeutic agents and other anti-neoplastics, antibiotics, anti-virals, anti-fungals, anti-inflammatories, anticoagulants, an antigenic materials.

The container may furthermore comprise one or more pharmaceutical acceptable carriers. Pharmaceutically acceptable carriers may be prepared from a wide range of materials. Without being limited thereto, such materials include diluents, binders and adhesives, lubricants, disintegrants, colorants, bulking agents, flavorings, sweeteners, and miscellaneous materials such as buffers and absorbents.

Further examples of medicaments according to the present invention are antimicrobial agents, analgesics, antiinflammatory agents, counterirritants, coagulation modifying agents, diuretics, sympathomimetics, anorexics, antacids and other gastrointestinal agents, antiparasitics, antidepressants, antihypertensives, anticholinergics, stimulants, antihormones, central and respiratory stimulants, drug antagonists, lipid-regulating agents, uricosurics, cardiac glycosides, electrolytes, ergot and derivatives thereof, expectorants, hypnotics and sedatives, antidiabetic agents, dopaminergic agents, anti-emetics, muscle relaxants, para-sympathomimetics, anticonvulsants, antihistamines, β-blockers, purgatives, antiarrhythmics, contrast materials, radiopharmaceuticals, antiallergic agents, tranquilizers, vasodilators, antiviral agents, and antineoplastic or cytostatic agents or other agents with anticancer properties, or a combination thereof. Other suitable medicaments may be selected from contraceptives and vitamins as well as micro- and macronutrients.

Further therapeutic agents which may be administered in accordance with the present invention include, without limitation: antiinfectives such as antibiotics and antiviral agents; analgesics and analgesic combinations; anorexics; antihelmintics; antiartritics; antiasthmatic agents; anticonvulsants; antidepressants; antidiuretic agents; antidiarrleals; antihistamines; antiiflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

Further specific examples of bioactive substances that can be stored in the containers in accordance with the present invention include acebutolol, acetaminophen, acetohydoxamic acid, acetophenazine, acyclovir, adrenocorticoids, allopurinol alprazolam, aluminum hydroxide, amantadine, ambenonium, amiloride, aminobenzoate; potassium, amobarbital, amoxicillin, amphetamine, ampicillin, androgens, anesthetics, anticoagulants, anticonvulsants-dione type, antithyroid medicine, appetite suppressants, aspirin atenolol, atropine, azatadine, bacampicillin, baclofen, beclomethasone, belladonna, bendroflumethiazide, benzoyl peroxide, benzthiazide, benztropine, betamethasone, betha nechol, biperiden, bisacodyl, bromocriptine, bromodiphenhydramine, brompheniramine, buclizine, bumetamide busulfan, butabarbital, butaperazine, caffeine, calcium carbonate, captopril, carbamazepine, carbenicillin, carbidopa & levodopa, carbinoxamine inhibitors, carbonic anhydsase, carisoprodol, carphenazine, cascara, cefaclor, cefadroxil, cephalexin, cephradine, chlophedianol, chloral hydrate, chlorambucil, chloramphenicol, chlordiazepoxide, chloroquine, chlorothiazide, chlorotrianisene, chlorpheniramine, <a6X chlorpromazine, chlorpropamide, chlorprothixene, chlorthalidone, chlorzoxazone, cholestyramine, cimetidine, cinoxacin, clemastine, clidinium, clindamycin, clofibrate, clomiphere, clonidine, clorazepate, cloxacillin, colochicine, coloestipol, conjugated estrogen, contraceptives, cortisone, cromolyn, cyclacillin, cyclandelate, cyclizine, cyclobenzaprine, cyclophosphamide, cyclothiazide, cycrimine, cyproheptadine, danazol, danthron, dantrolene, dapsone, dextroamphetamine, dexamethasone, dexchlorpheniramine, dextromethorphan, diazepan, dicloxacillin, dicyclomine, diethylstilbestrol, diflunisal, digitalis, diltiazen, dienhydrinate, dimethindene, diphenhydramine, diphenidol, diphenoxylate & atrophive, diphenylopyraline, dipyradamole, disopyramide, disulfiram, divalporex, docusate calcium, docusate potassium, docusate sodium, doxyloamine, dronabinol ephedrine, epinephrine, ergoloidmesylates, ergonovine, ergotamine, erythromycins, esterified estrogens, estradiol, estrogen, estrone, estropipute, etharynic acid, ethchlorvynol, ethinyl estradiol, ethopropazine, ethosaximide, ethotoin, fenoprofen, ferrous fumarate, ferrous gluconate, ferrous sulfate, flavoxate, flecainide, fluphenazine, fluprednisolone, flurazeparn folic acid, furosemide, gemfibrozil, glipizide, glyburide, glycopyrrolate, gold compounds, griseofiwin, guaifenesin, guanabenz, guanadrel, guanethidine, halazepam, haloperidol hetacillin, hexobarbital, hydralazine, hydrochlorothiazide, hydrocortisone (cortisol), hydroflunethiazide, hydroxychloroquine, hydroxyzine, hyoscyamine, ibuprofen, indapamide, indomethacin, insulin, iofoquinol, ironpolysaccharide, isoetharine, isoniazid, isopropamide, isoproterenol, isotretinoin, isoxsuprine, kaolin & pectin, ketoconazole, lactulose, levodopa, lincomycin liothyronine, liotrix, lithium, loperamide, lorazepam, magnesium hydroxide, magnesium sulfate, magnesium trisilicate, maprotiline, meclizine, meclofenamatei medroxyproyesterone, melenamic acid, melphalan, mephenyloin, mephobarbital, meprobamate, mercaptopurine, mesoridazine, metaproterenol, metaxalone, methaniphetami e, methaqualone, metharbital, methenamine, methicillin, methocarbamol, methotrexate, methsuximide, methyclothizide, methylcellulos, methyldopa, methylergonovine, methylphenidate, methylprednisolone, methysergide, metoclopramide, metolazone, metoprolol, metronidazole, minoxidil, mitotane, monamrine oxidase inhibitors, nadolol, nafcillin, nalidixic acid, naproxen, narcotic analgesics, neomycin, neostigmine, niacin; nicotine, nifedipine, nitrates, nitrofrantoin, nomifensine, norethindrone, norethindrone acetate, norgestrel, nylidrin, nystatin, orphenadrine, oxacillin, oxazepam, oxprenolol, oxymetazoline, oxyphenbutazone, pancrelipase, pantothenic acid, papaverine, para-aminosalicylic acid, parmethasone, paregoric, pemoline, penicillamine, penicillin, penicillin-v, pentobarbital, perphenazine, phenacetin, phenazopyridine, phemrarnne, phenobarbital, phenolphthalein, phenprocoumon, phensuximide, phenylbutazone, phenylephrine, phenylpropanolamine, phenyl toloxamine, phenyloip, pilocarpine, pindolol, piper actezine, piroxicam, poloxamer, polycarbophil calcium, polythiazide, potassium supplements, pruzepam, prazosin, prednisolone, prednisone, primidone, probenecid, probucol, procainamide, procarbazine; prochlorperazine, procyclidine, promazine, promethazine, propantheline, propranolol, pseudoephedrine, psoralens, syllium, pyridostiginine, pyrodoxine, pyrilamine, pyrvinium, quinestrol, quiriethazone, ninidine, quinine, ranitidine, rauwolfia alkaloids, riboflavin, rifampin, ritodrine, alicylates, scopolamine, secobarbital, senna, sannosides a & b, simethicone, sodium bicarbonate; sodium phosphate, sodium fluoride, spironolactone, sucrulfate, sulfacytine, sulfamethoxazole, sulfasalazine, sufinpyrazone, sulfisoxazole, suindac, talbutal, tanazepam terbutaline, terfenadine, terphinhydrate, teracycines, thiabendazole, thiamine, thioridazine, thiothixene, thyroblobulin, thyroid, thyroxine, ticarcillin timolol, tocainide, tolazamide, tolbutamide, tolmetin trozodone, tretinoin, triamcinolone, trianterene, triazolam, trichlormethiazide, tricyclic antidepressants, tridhexethyl, trifluoperazine, triflupromazine, trihexyphenidyl, trimeprazine, trimethobenzamine, trimethoprim, tripclennamine, triprolidine, valproic acid, verapamil, vitamin A, vitamin B-12, vitamin C, vitamin. D, vitamin E, vitamin K, xanthine, and the like.

The containers are also suitable for the storage of polypeptides, for example hormones such as growth hormones, enzymes such as lipases, proteases, carbohydrases, amylases, lactoferrin, lactoperoxidases, lysozymes, nanoparticles, etc., and antibodies. The container may also be employed for the storage of microorganisms, either living, attenuated or dead, for example bacteria, e.g. gastrointestinal bacteria such as streptococci, e.g. S. faecium, Bacillus spp. such as B. subtilis and B. licheniformis, lactobacteria, Aspereillus spp., bifidogenic factors, or viruses such as indigenous vira, enterovira, bacteriophages, e.g. as vaccines, and fungi such as baker's yeast, Saccharomyces cerevisiae and fingi imperfecti.

The contianer may also be used for the storage of active agents in specialized carriers such as liposomes, cyclodextrines, nanoparticles, micelles and fats.

Further examples of medicaments capable of being stored in a container according to the invention include, but are not limited to, antihistamines (e.g., dimenhydrinate, diphenhydramine (50-100 mg), chlorpheniramine and dexchlorpheniramine maleate), analgesics (e.g., aspirin, codeine, morphine (15-300 mg), dihydromorphone, oxycodone, etc.), anti-inflammatory agents (e.g., naproxyn, diclofenac, indomethacin; ibuprofen, acetaminophen, aspirin, sulindac), gastro-intestinals and anti-emetics (e.g., metoclopramide (25-100 mg)), antiepileptics (e.g., phenytoin, meprobamate and nitrazepam), vasodilators (e.g., nifedipine, papaverine, diltiazem and nicardipine), antitussive agents and expectorants (e.g., codeine phosphate), anti-asthmatics (e.g. theophylline), anti-spasmodics (e.g. atropine, scopolamine), hormones (e.g., insulin, heparin), diuretics (e.g., ethacrynic acid, bendroflumethiazide), anti-hypotensives (e.g., propranolol, clonidine), bronchodilators (e.g., albuterol), anti-inflammatory steroids (e.g., hydrocortisone, triamcinolone, prednisone), antibiotics (e.g., tetracycline), anti-hemorrhoidals, hypnotics, psychotropics, antidiarrheals, mucolytics, sedatives, decongestants, laxatives, antacids, vitamins, stimulants, (including apetite suppressants such as phenylpropanolamine). The above list is not meant to be exclusive.

Other types of medicaments include flurazepam, nimetazepam, nitrazepam, perlapine, estazolam, haloxazolamn, sodium valproate, sodium cromoglycate, primidone, alclofenac, perisoxal citrate, clidanac, indomethacin, sulpyrine, flufenamic acid, ketoprofen, sulindac, metiazinic acid, tolmetin sodium, fentiazac, naproxen, fenbufen, protizinic acid, pranoprofen, flurbiprofen, diclofenac sodium, mefenamic acid, ibuprofen, aspirin, dextran sulfate, carindacillin sodium, and the like.

The medicament may be in the form of a physiologically active polypeptide, which is selected from the group consisting of insulin, somatostatin, somatostatin derivatives, growth hormone, prolactin, adrenocorticotrophic hormone, melanocyte stimulating hormone, thyrotropin releasing hormone, its salts or its derivatives, thyroid stimulating hormone, luteinizing hormone, follicle stimulating hormone, vasopressin, vasopressin derivatives, oxytocin, carcitonin, parathyroid hormone, glucagon, gastin, secretin, pancreozymin, cholecystokinin, angiotensin, human placental lactogen, human chorionic gonadotropin, enkephalin, enkephalin derivatives, endorphin, interferon (in, one or more of the forms alpha, beta, and gamma), urokinase, kallikrein, thymopoietin, thymosin, motilin, dynorphin, bombesin, neurotensin, caerulein, bradykinin, substance P, kyotorophin, nerve growth factor, polymyxin B, colistin, gramicidin, bacitracin, bleomycin and neocarzinostatin. Furthermore, the medicament may be a polysaccharide, such as heparin, an antitumor agent such as lentinan, zymosan and PS-K (krestin), an aminoglycoside such as e.g. gentamycin, streptomycin, kanamycin, dibekacin, paromomycin, kanendomycin, lipidomycin, tobramycin, amikacin, fradiomycin and sisomicin, a beta-lactam antibiotic, such as e.g. a penicillin, such as e.g. sulbenicillin; mecillinam, carbenicillin, piperacillin and ticarcillin; thienamycin, and cephalosporins such as cefotiam, cefsulodine, cefmenoxime, cefmetazole, cefazolin, cefotaxime, cefoperazone, ceftizoxime and moxalactam, or a nucleic acid drug such as e.g. citicoline and similar antitumor agents, for example cytarabine and 5-FU (5-fluorouracil).

Certain monomeric subunits of the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as failing within the scope of the invention. Additional asymmetric carbon atoms may be present in a substituent such as an alkyl group. All such isomers, as well as mixtures thereof, are intended to be included in this invention,

For the purposes of this application, unless expressly noted to the contrary, a named amino acid shall be construed to include both the D or L stereoisomers, preferably the L stereoisomer.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic means well known in the art, and subsequent recovery of the pure enantiomers.

### EXAMPLES

The following examples are illustrative of the present invention and will explain the invention in a non-limiting way.

### Example 1

Synthesis of α-4-azidobenzoyl ω-methoxy poly(ethylene glycol)s (ABMPEG)

The synthesis of photo-reactive ABMPEG 5 kDa is described. ABMPEG of different MWs (2, 5, and 10 kDa) were employed as modifying agents in all following examples, all being synthesized as described in this example.

1. Procedure

4-Azidobenzoic acid is prepared from 4-aminobenzoic-acid which is diazotized with sodium nitrate.^{[39,40]} The carboxylic acid is converted into the 4-azido benzoyl chloride with thionyl chloride.^{[39,40]}0.23 g (1.875 mmol) of dimethylaminopyridine (DMAP) in 10 ml dry methylene chloride is mixed with 0.17 ml (1.250 mmol) triethylamine (TEA). The solution is transferred into a 250 ml three neck roundbottom flask. After cooling down to 0° C., 0.57 g (3.125 mmol) 4-azido benzoyl chloride in 10 ml CH₂Cl₂ is added forming a yellow dispersion. 6.25 g (1.5 mmol) MPEG 5 kDa in 50 ml dry CH₂Cl₂ is added dropwise during 1 hour under dry nitrogen, after which the temperature is allowed to rise to room temperature. The reaction is continued with stirring overnight. The solution is filtered, and ABMPEG is precipitated in cold diethylether. The product is purified by two further precipitations from CH₂Cl₂/diethylether and dried in vacuum. Yield: 4.83 g (74%)

### Example 2

Adsorption Characteristics/Kinetics of ABMPEG 5 kDa and MPEG 5 kDa to a Polysulfone Surface Monitored by Ellipsometry

Ellipsometry is a very sensitive technique for the determination of adsorption kinetics to optically smooth surfaces. For better resolution, transparent polysulfone (PSf) films were spin-coated onto polished silicon wafers, and thus the reflecting properties of the underlying silicon were exploited

1. Preparation of PSf Surfaces

Hydrophilic silicon slides: Silica surfaces are prepared from polished silicon wafers which are thermally oxidized in pure and saturated oxygen followed by annealing and cooling under argon flow to yield an oxide layer of about 30 nm. Wafers are cut into rectangular slides (10-14 mm×20-30 mm), thoroughly cleaned with detergent, etched for 15 min in a freshly mixed 3:1 (v:v) sulfuric acid (96%): hydrogen peroxide (30%) solution, thoroughly rinsed, stabilized for 2 hours and rinsed again with/in ultrapure water. Slides are dried free of dust for two hours at 120° C. This procedure results in surfaces dense in silanol groups with a contact angle of less than 10°.
Hydrophobic silicon slides: In order to yield hydrophobic surfaces, previously prepared hydrophilic silicon slides are silanised in air saturated with hexamethyldisilazane (HMDS) at approx. 110° C. Excess HMDS is rinsed away with ultrapure H₂O. Slides are dried free of dust at room temperature.
PSf-spin-coated hydrophobic silicon slides: The previously prepared hydrophobic silicon slides are spin-coated with a 3% (w:w) PSf in 1,2-dichlorbenzene solution. Slides are completely wetted by the polymer solution and then spun for 10 sec at 500 rpm and consecutively for 50 sec at 5.000 rpm in order to attain a smooth polymer film. Coated slides are dried for at least 4 hours at vacuum at 60° C.
   2. Ellipsometry Measurements

ABMPEG 5 kDa and mono-methoxy-PEG MPEG 5 kDa adsorption out of aqueous solution to PSf spin-coated HMDS-treated silicon slides is monitored in suit using an automated Rudolph Thin Film ellipsometer, type 43603-200E, equipped with a thermostated quartz cuvette.^{[38]} Spin-coated slides are stabilized in 4.5 ml water for at least 15 min or until constant polarizer and analyzer signals are obtained. 0.5 ml of concentrated aqueous ABMPEG 5 kDa/MPEG 5 kDa solution is added yielding 5 ml solution at defined concentration. A magnetic stirrer is activated for 30 sec upon addition of the ABMPEG 5 kDa/MPEG 5 kDa concentrate in order to homogenize the solution. Polarizer and analyzer data is collected until apparent equilibrium is reached. From the attained data, it is possible to calculate thickness and refractive index of an adsorbed layer and/or its mass.^{[41]} Adsorption data is calculated for approximated values of the partial specific volume and the ratio between the molar weight and the molar refractivity for both ABMPEG 5 kDa and MPEG 5 kDa respectively applying the same values for both species. Results for the calculated adsorbed mass are represented in arbitrary units as only approximated values of the partial specific volume and molar refractivity of ABMPEG 5 kDa and MPEG 5 kDa were at hand.

3. Results

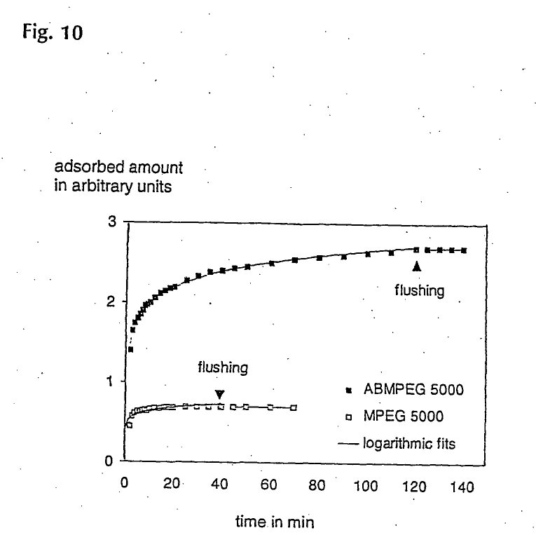

FIG. 10 depicts adsorption kinetics monitored by ellipsometry for ABMPEG 5 kDa and MPEG 5 kDa respectively. Enhanced adsorption (factor =3.5) and prolonged equilibrium times (>2 h) are observed for ABMPEG 5 kDa when compared with MPEG 5 kDa. The pronounced difference in the adsorptive characteristics of the two materials indicates a strong affinity between the hydrophobic (aromatic) head-group of ABMPEG 5 kDa and the hydrophobic PSf surface. This affinity leads to an oriented layer, were the headgroup is in close contact with the underlying substratum and thus very well positioned to be, effectively grafted through photo-activation. Furthermore, flushing with water (20 ml/min) does not effect the adsorbed amount, i.e. no desorption, neither of ABMPEG 5 kDa nor of MPEG 5 kDa, takes place. A similar behavior is to be expected also for ABMPEG of other MW, e.g. 2, or 10 kDa. In conclusion, this example illustrates how the photo-reactive headgroup of ABMPEG enhances the attractive interactions with a hydrophobic interface leading to increased adsorption in comparison to the non-conjugated MPEG.

### Example 3

Controlling Polymer Surface Hydrophilicity and Heterogeneity through Photo-Grafting of ABMPEG

PSf spin-coated films on glass coverslips were modified with ABMPEG 2, 5, and 10 kDa Desired degrees of hydrophilicity and thus surface density of the different ABMPEG on PSf were attained by adjusting bulk ABMPEG concentrations during a first adsorptive step. Contact angles (CA) were used to monitor resulting changes. Mixtures of different ABMPEG were applied in order to attain intermediate surface characteristics. The effectiveness of the photoreactive grafting was evaluated for ABMPEG 10 kDa by removing non-grafted ABMPEG moities.

1. Preparation of Polymer-Surfaces

Glass coverslips are cleaned with detergent, rinsed with ultrapure water, and etched for 15 min at approx. 40±5° C. in a freshly mixed 3:1 (v:v) sulfuric acid (96%): hydrogen peroxide (30%) solution. Coverslips are thoroughly rinsed, stabilized for 2 hours and rinsed again with/in ultrapure H₂O. Slips are dried free of dust for two hours at 120° C. n-octadecyldimethylchlorosilane (ODDMS) is grafted to the cleaned coverslips by immersing them in a 2% (w:w) ODDMS in n-hexane solution for 1 hour at room temperature. Coverslips are rinsed twice with n-hexane and three times with ethanol and air dried at room temperature. The ODDMS-treated coverslips are spin-coated with a 3% (w:w) PSf in 1,2-dichlorbenzene solution. Coverslips are completely wetted by the polymer solution and then spun for 10 see at 500 rpm and consecutively for 50 sec at 5.000 rounds per minute (rpm) in order to attain a smooth polymer film. Coated coverslips are dried for at least 4 hours at vacuum at 60° C.

2. ABMPEG Grafting to Polymer Surface

ABMPEG grafting includes the following two consecutive steps as illustrated in FIG. 4. In a first adsorption step aqueous ABMPEG solution of different concentrations is placed on the PSf coated coverslip, covered and kept in the dark for at least 12 h but maximal 18 h. Thereafter coverslips are gently rinsed in ultrapure water, covered by water and immediately exposed to UV light for 1 min. For TV irradiation a 50 W high pressure mercury lamp (ORIEL) equipped with a condenser is used. The UV rich light passes a high-pass glass filter with a cut off at 320 nm yielding an intensity of 30 mW/cm². Certain indicated control surfaces are not exposed to UV irradiation. To remove non-covalently bond ABMPEG certain indicated sample surfaces were exposed over night to a 1:1 (v:v) water:isopropanol mixture (H₂O/IP), thoroughly rinsed with the same mixture and with ultrapure water thereafter.

3. Contact Angle (CA) Measurements

As on modified and unmodified PSf coated coverslips are measured using the captive bubble method where an air bubble is injected from a syringe with a stainless steel needle onto the inverted sample surfaces under water. The diameter of the contact area between the PSf and the bubbles is always greater than 3 mm. While the needle remains inside the bubble, advancing and receding angle measurements are realized with a goniometer fitted with a tilting stage by stepwise withdrawing/adding air from/to the captured bubble. At least ten measurements of different bubbles on at least three different locations are averaged to yield one data.

4. Results

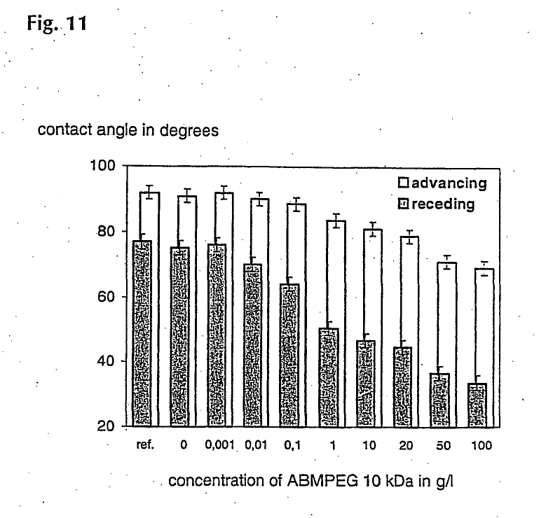

FIG. 11 shows advancing and receding As of PSf spin-coats modified with different concentrations of ABMPEG 10 kDa. Surfaces were exposed to UV irradiation but not rinsed with (H₂O/IP). Note that under the valid assumption that adsorbed ABMPEG layers are in the relevant time scales stable in aqueous environment, i.e. no desorption will take place (see results in Example 2), ABMPEG 10 kDa adsorption is monitored and not its chemical grafting. With rising ABMPEG 10 kDa bulk concentrations de, creasing advancing and receding As are observed while CA-hysteresis increases in the applied concentration range. The results indicate that ABMPEG. 10 kDa adsorption is highly controllable and reproducible. Desired degrees of hydrophilicity and thus surface density of ABMPEG 10 kDa are attained by adjusting bulk ABMPEG. 10 kDa concentrations during adsorption

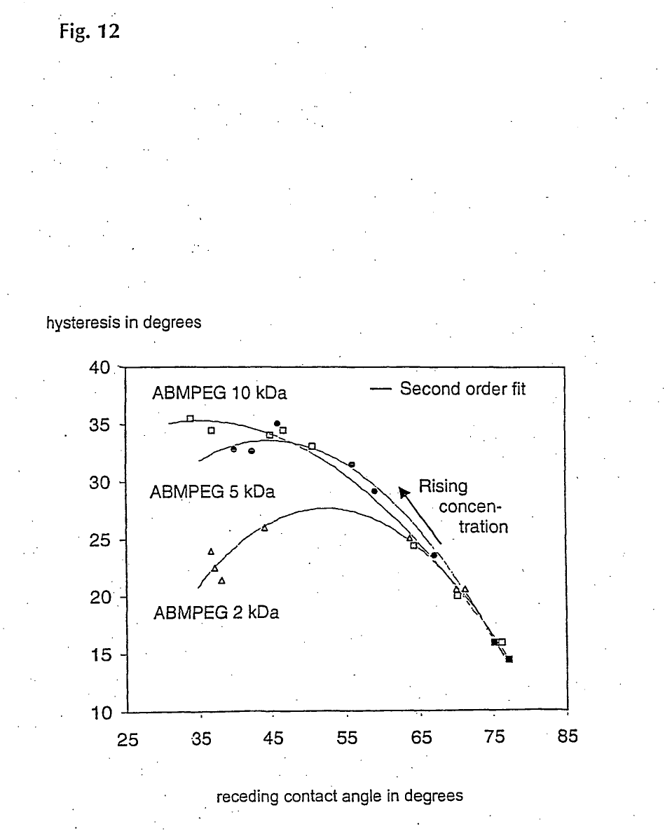

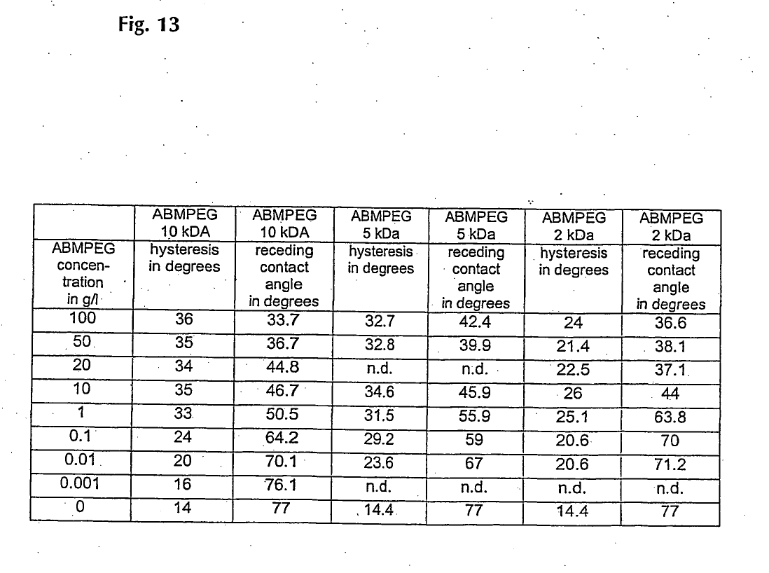

FIG. 12 shows As of surfaces which were modified applying ABMPEG of three different chain lengths, i.e. three different MWs: 2, 5, and 10 kDa (see also FIG. 13). Again, surfaces were exposed to UV irradiation but not rinsed with H₂O/IP thereafter. The same trend regarding degree and controllability of the attained hydrophilization of the underlying PSf is observed for all different chain lengths, but differences in CA-hysteresis are observed CA-hysteresis values are in general lower for shorter chain lengths, and a clear maximum is seen especially for the lowest MW ABMPEG in the applied concentration range. Thus longer chain lengths seem to induce more chemical and/or morphological heterogeneity manifested in increased CA-hysteresis.

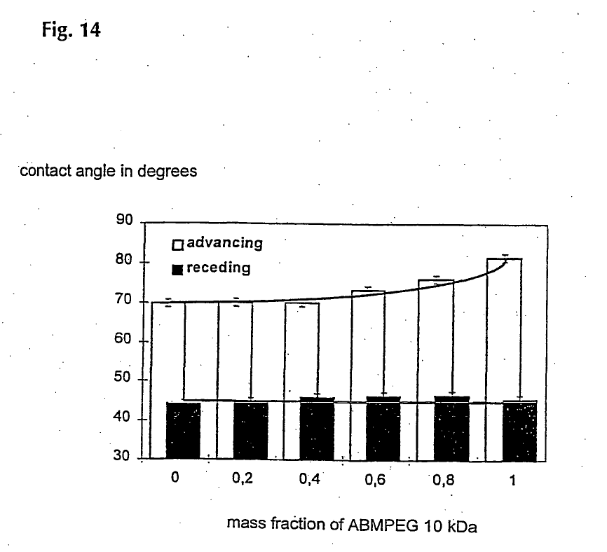

FIG. 14 shows receding As and. CA-hysteresis of PSf surfaces, which were modified with different mixtures of ABMPEG of two different chain lengths (ABMPEG 2 kDa and ABMPEG 10 kDa). Again, surfaces were exposed to UV irradiation but not rinsed with H₂O/IP thereafter. Surfaces show a gradual change in surface properties. This result implies that mixtures of different ABMPEG and/or ABMPEG derivatives can be applied in order to attain/design intermediate surface characteristics.

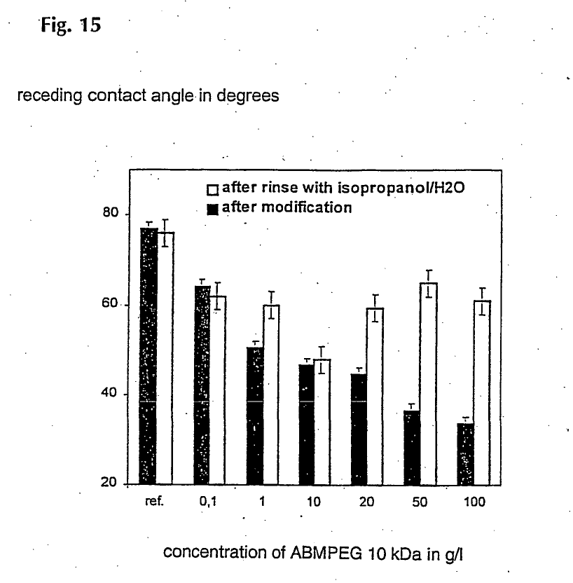

FIG. 15 shows receding As of PSf surfaces modified with different concentrations of ABMPEG 10 kDa. Samples were exposed to UV irradiation and the As measured before and after over night rinsing with H₂O/IP. The data characterizes the efficiency of the photo-grafting process in dependence of applied ABMPEG concentration. For ABMPEG concentrations higher than 10 g/l the effectiveness of the photoreactive grafting diminishes rapidly manifested in the reversibility of the hydrophilization upon rinsing with H₂O/IP. This indicates a decrease in head-group orientation towards the surface lowering chances for successful grafting. Increased solute-solute interactions at rising surface coverage might be responsible.

### Example 4

Assay of Protein Adsorption to Modified PSf Membranes

Commercially available standard ultrafiltration membranes were modified at different degrees of modification with ABMPEG 5 kDa. Thereafter, the adsorptive properties of the membranes were evaluated by exposing them to a buffered solution of bovine serum albumine (BSA) and determining the adsorbed amount of BSA.

1. Membrane Modification

Rinsed pieces of cot cut PSf ultrafiltration membrane (132.6 cm², type GR61PP, DOW, Danmark) ( ) were stabilized and cleaned from packaging liquids by permeating at least 6 liter of ultrapure water at 0.4 MPa for at least 1 h. The membrane was then cut into circular membranes of 25 mm diameter and their skin-layer modified with ABMPEG 5 kDa as described in Example 3. After exposure to 7UV irradiation were membranes exposed to ultrasound for 5 min and thoroughly rinsed thereafter.

2. Protein Adsorption

The skin-layer of unmodified or modified membranes was contacted for 2 hours with a 1 g/l BSA solution (0.15 molar phosphate buffer, pH=7, room temperature), flushed with buffer and dried at 60° C. over night. Adsorbed amount of BSA is determined by its total hydrolysis and consecutive amino acid analysis.^{[42]}

3. Results

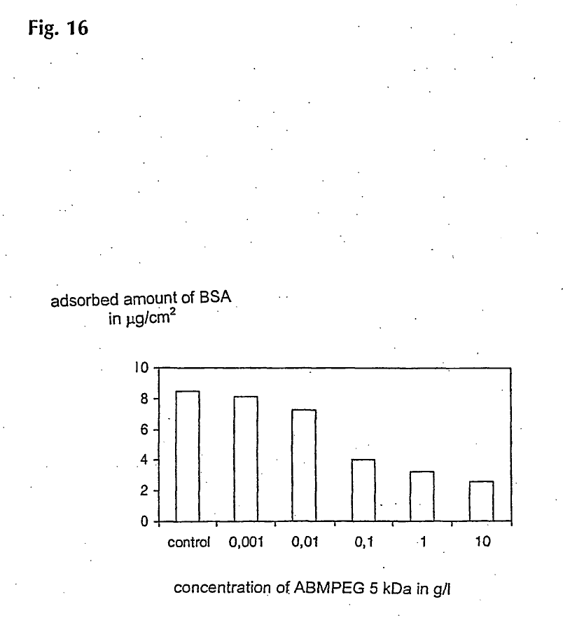

FIG. 16 shows adsorbed amounts of BSA in dependence of the applied ABMPEG 5 kDa concentration. BSA adsorption decreases for increasing ABMPEG 5 kDa concentration. Maximum reduction in comparison to an unmodified reference membrane of about 70% is attained for the highest applied ABMPEG 5 kDa concentration of 10 g/l.

### Example 5

Fibronectin (FN) Adsorption to Unmodified and ABMPEG 10 kDa Modified PSf as Measured by in situ Ellipsometry

As in Example 3, the reflecting properties of polished silicon were exploited to monitor FN adsorption to unmodified or mod ed spin-coated PSf film. Adsorption kinetics of FN to the differently modified surfaces are attained yielding information about the interfacial interactions of FN with the photo-grafted ABMPEG 10 kDa interfacial structure.

1. Procedure

Fibronectin (ET) (human plasma, lyophilized, MW 440 kDa; Boehringer Mannheim, Germany) is reconstituted in phosphate-buffered saline (PBS; 5.8 mM Na₂HPO₄/NaH₂PO₄, 150 mM NaCl, pH=7.4) containing 0.02% (w/v) sod azide giving a concentration of about 0.12 g/l. The instrumental setup and the measurement procedure are identical to the one described in Example 2.1 and 2.2 respectively. PSf films on silicon wafers, modified with ABMPEG 10 kDa at different concentrations as described in Example 3.1 are placed in the quartz cuvette and stabilized in 2.5 ml PBS buffer for at least 15 min or until constant polarizer and analyzer signals are obtained. 0.5 ml of the concentrated FN solution is added yielding 3 ml with a defined concentration of 0.02 g/l. The magnetic stirrer is activated for 2-3 sec upon addition of the protein concentrate in order to homogenize the solution. After 30 min the cuvette is flushed for 10 min with PBS buffer using preinstalled tubings and a flow rate of 20 ml/min. Even if plateau values are typically observed after 1-2 hours (or much longer), it is possible to describe protein—substratum interactions also already after 30 min.

In the calculations of the amount of protein adsorbed, the different layers, silicon support, silicon oxide, ODMS-layer, PSf-film, and tethered ABMPEG 10 kDa are treated as one optical unit with an effective refractive index. The molar refractivity of FN is calculated as the sum of the individual molar refractivities of all amino acids in FN using tabulated values^{[43]} yielding a value of 3.99 g/ml. For the partial specific volume of FN the value 0.75 ml/g is used.

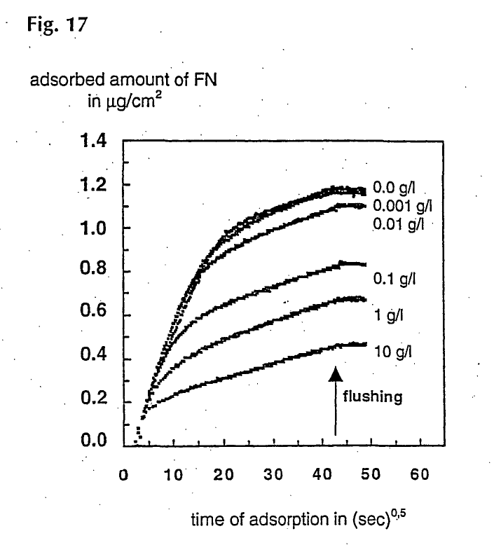

FIG. 17 shows that all data curves follow the expected monotonic rise; FN desorption upon flushing is not observed. The adsorbed amount of FN decreases with higher degrees of ABMPEG 10 kDa surface functionalization and thus correlates qualitatively with the CA decrease as shown in FIG. 11. Maximum adsorption of almost 1.2 μg/cm² is attained for both, unmodified PSf and PSf modified with the lowest ABMPEG 10 kDa concentration, i.e. 0.001 g/l. The adsorbed FN amount decreases by more than 60% to 0.45 μg/cm² for an ABMPEG 10 kDa concentration of 10 g/l. As shown in Example 4, BSA adsorption to PSf UF membrane surfaces photo-grafted with ABMPEG 5 kDa and quantified by total hydrolysis and consecutive amino acid analysis of the adsorbed protein yielded very similar results: The relative reduction depending on the degree of functionalization correlates very well with the here presented results for FN.

### Example 6

Fibroblast Adhesion to Unmodified and ABMPEG 10 kDa Modified PSf Surfaces: Overall Cell Morphology, Number of Adherent Cells, and Focal Adhesion Formation

The number of adherent cells, the overall cell morphology, and the development of focal adhesions are good indications for the quality of interactions between cells and interfaces. Many previous studies have shown, that the more cells adhere, the more spread the cells are, and the more pronounced focal adhesions are formed, the better suited are the respective surfaces for the anchorage and proliferation of the investigated cells.

1. Cells

Human fibroblasts (HF) were obtained from fresh skin biopsy and used up to the 9th passage. The cells were grown in Dulbecco's Modified Eagle Medium (DMEM) containing 10% fetal bovine serum (FBS, Sigma Chemicals Co., St. Louis, Mo., USA) in an humidified incubator with 5% CO₂. HF from nearly confluent cultures were harvested with 0.05% trypsin/0.6 mM EDTA (Sigma), and trypsin was neutralized with FBS.

2. Number of Adherent HF and their Morphology

Adhesion of BF was carried out in 6-well tissue culture plates containing the unmodified and ABMPEG 10 kDa modified PSf coated glass slides. Experiments were performed without or with pre-coating of the surfaces with FBS (Sigma) for 30 min at 37° C. Approximately 5*10⁵ cells in DMEM were pipetted into each well and incubated for 2 h at 37° C. in a humidified CO₂ incubator. The number of adherent cells and their morphology was studied and photographed directly from the wells with an inverted phase contrast microscope Telaval 31 (Carl Zeiss, Germany). The mean number of adherent cells was determined by evaluating approx. 30 different randomly chosen microscopic fields on each surface. Cell counts were normalized to: number of cells per area of microscopic field; the standard deviation was determined for each set of fields on a surface.

3. Focal Adhesions Formation

Focal adhesions of HF plated on non-precoated and serum-precoated substrata were visualized by immunofluorescence. Samples were processed as follows: Attached cells were fixed with paraformaldehyde (3%) for 10 min and permeabilized with 0,2% Triton X-100 for 5 min. To detect focal adhesions, samples were incubated for 30 min at 37° C. with monoclonal anti vinculin antibody (Sigma Immunochemicals, St. Louis, Mich., USA), followed by Cy3 conjugated goat anti mouse secondary antibody (Jackson Immuno Research, Inc. West Growe, Pa., USA). Samples were mounted with Mowiol, and viewed and photographed with a inverted fluorescent microscope Axiovert 100 (Carl Zeiss, Germany).

4. Results

Already shortly after plating cells (2 h), clear differences can be observed in dependence of the underlying substratum.

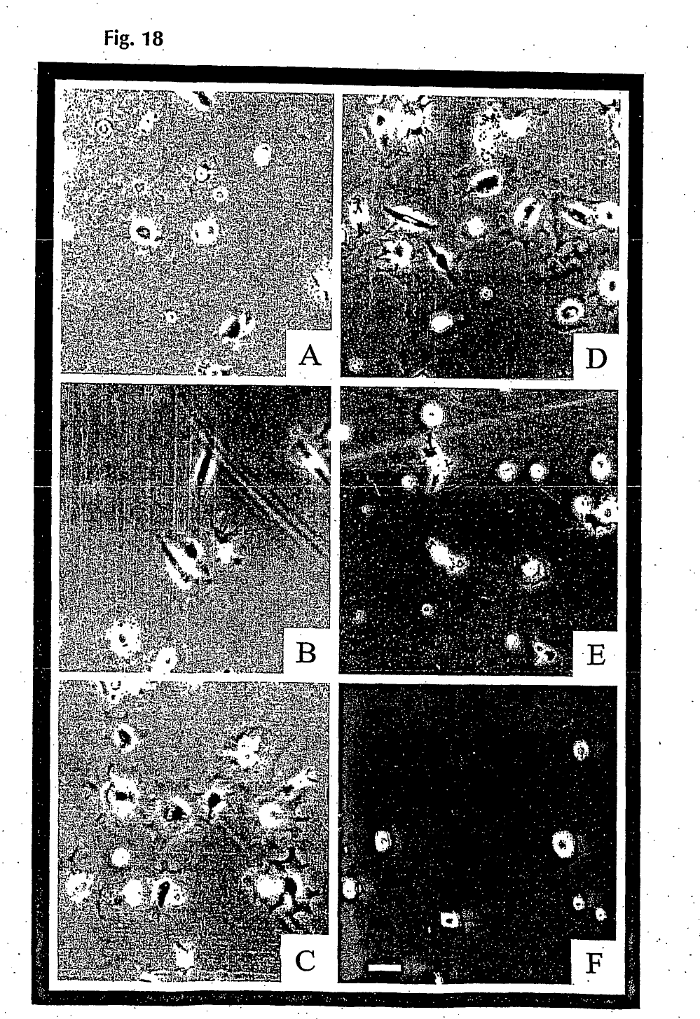

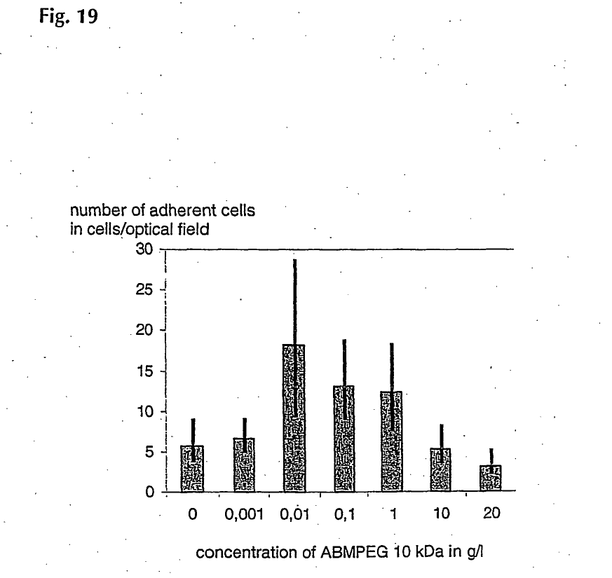

FIG. 18 shows the overall cell morphology of adherent HF. A clear dependence between the amount of adherent cells (see also FIG. 19) and their spreading and the employed ABMPEG 10 kDa concentrations can be seen in the phase-contrast pictures.

PSf modified at intermediate concentrations of ABMPEG 10 kDa (0.001-0.01) shows increasing adherence and spreading of the plated HF-cells.

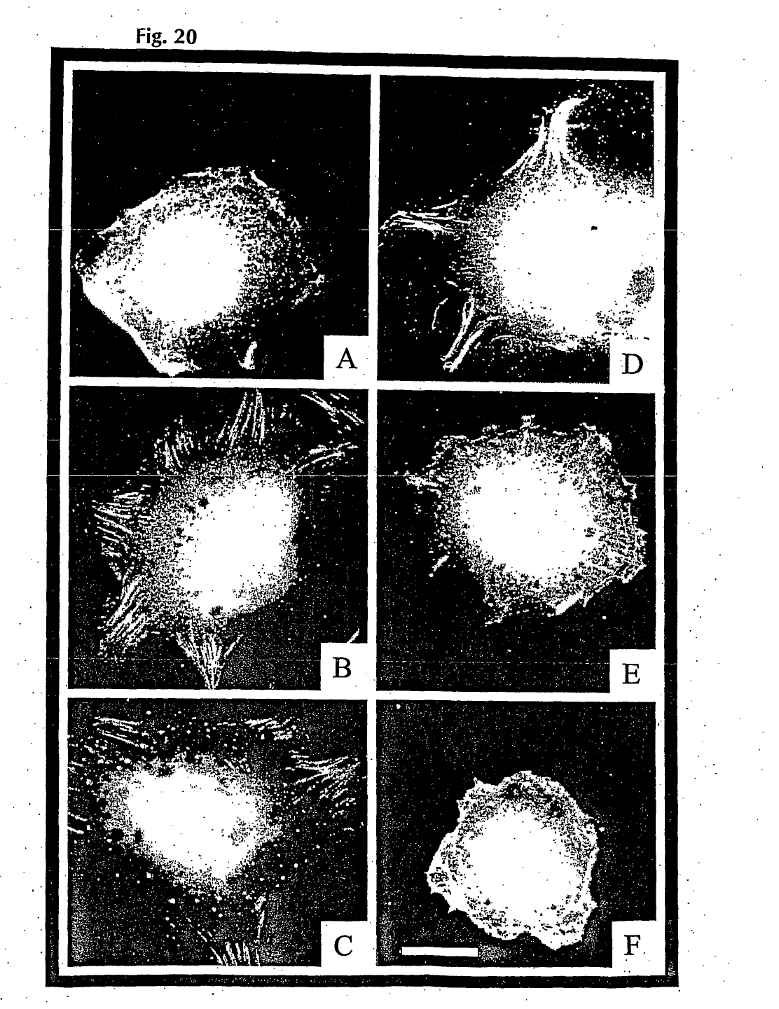

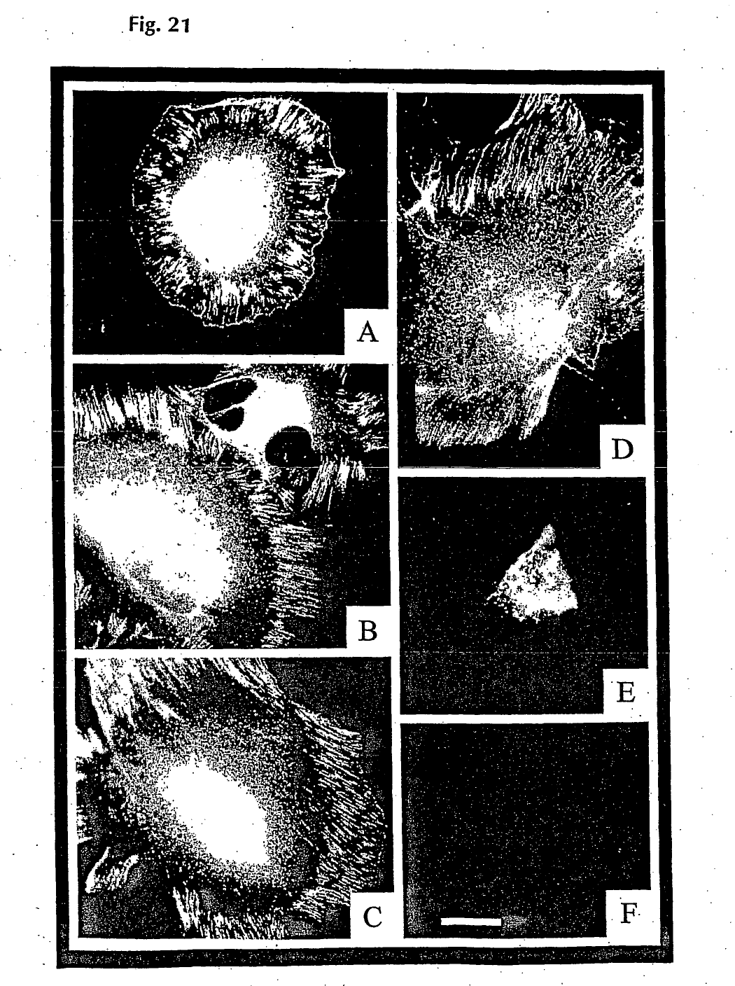

Focal adhesion formation on non-precoated substrata illustrated in FIG. 20 demonstrates again significantly improved cell morphology and spreading on PSf surfaces modified with intermediate concentration of ABMPEG 10 kDa (0.001 g/l and 0.01 g/l), in comparison to unmodified PSf, or PSf modified with relatively high concentrations of ABMPEG 10 kDa (1 g/l and 10 g/l). Focal adhesion formation on serum-precoated substrata illustrated in FIG. 21 represents the optimal focal adhesions formation on substrata modified with intermediate concentration of ABMPEG 10 kDa (0.001 g/l (13), and 0.01 g/l (C)). At 0.1 g/l (D), focal adhesions already start to disorganize and almost completely disappear at 10 g/l (F). An important observation is that the effect of ABMPEG density on focal adhesions formation is much more pronounced on serum coated ABMPEG surfaces (see FIG. 20, and compare with FIG. 21). Thus, modifying PSf with minute amounts of ABMPEG leads to much enhanced cell-substratum interactions.

### Example 7

Fibronectin Matrix Formation of Fibroblasts Adhering on Unmodified and ABMPEG. 10 kDa Modified PSf Surfaces

Fibronectin (FN) is an adhesive protein being essential for the adhesion/anchorage of cells to any kind of substratum. Shortly after contacting a suitable substratum, viable BF-cells will secret FN and will form a FN matrix. The amount of secreted FN and the structure of the consecutively formed matrix can be used to evaluate the quality of cell-substratum interactions.

1. FN Matrix Formation

Approximately 5*10⁵ HF in 3 ml medium containing 10% FBS were incubated for 5 days in 6-well tissue culture plates (Falcon, Becton Dickenson & Company, N.J., USA) containing the PSf coated and photo-modified glass slides. At the end of the incubation cells were fixed with 3% paraformaldehyde and FN matrix deposited on the different surfaces was visualized by immunofluorescence using a specific anti human FN matrix mouse monoclonal antibody (lot No. 0326, Immunotech SA, France), followed by Cy3-conjugated goat anti mouse secondary IgG1 antibody (Jackson Immuno Research, Inc. West Growe, Pa., USA). Further investigations and photography were carried out with an inverted fluorescence microscope as above.

2. Results

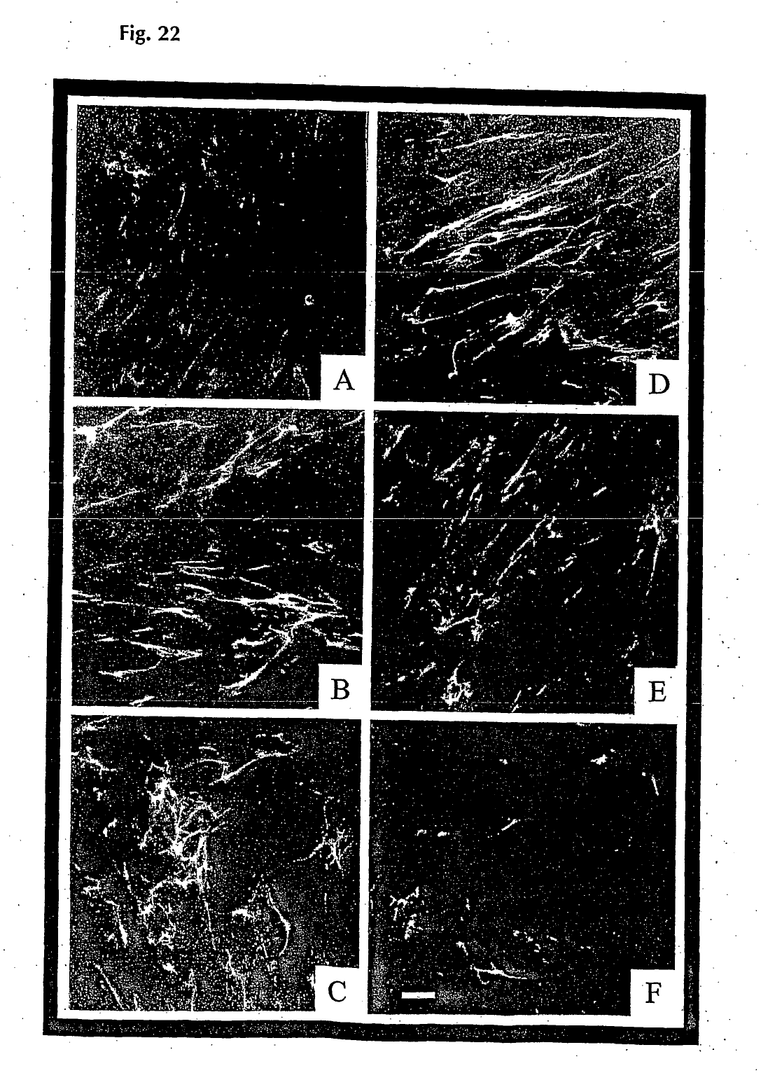

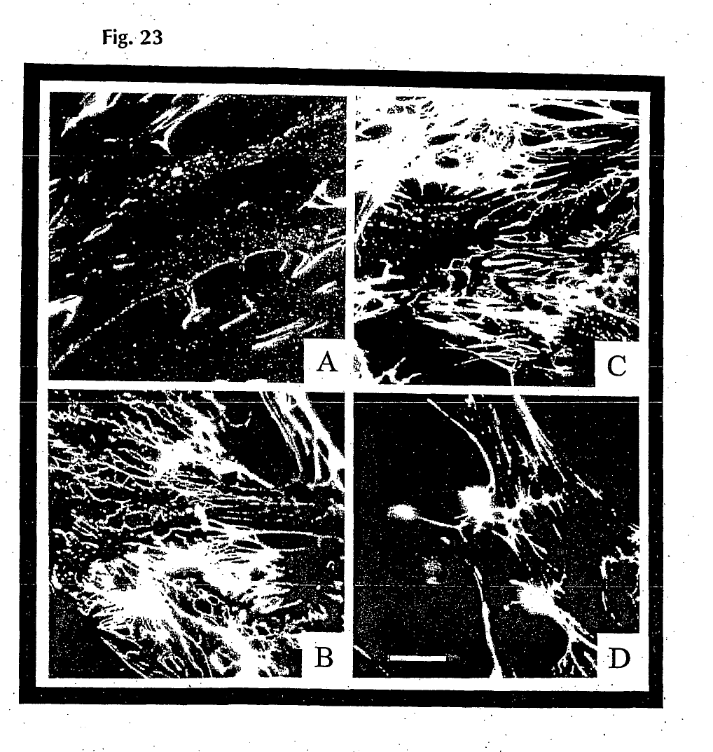

FIG. 22 demonstrates maximal FN matrix formation of HF cultured on surfaces with moderate ABMPEG 10 kDa density. Note, that the secreted FN was also highly organized on these surfaces (see FIG. 23).

### Example 8

Proliferation of Fibroblasts, Liver Cells, and Endothelial Cells on Unmodified and ABMPEG 10 kDa Modified PSf Surfaces

Phase-contrast photographs were executed in order to characterize overall cell morphology and proliferation of human fibroblasts (HF), liver cells (C3A), and human umbilical vein endothelial cells (HUVEC) on unmodified PSf as well as on ABMPEG 10 kDa modified PSf. Proliferation of BF was further characterized by semi* quantitative XTT and LDH assays which are established functional methods for cell proliferation. The XTT assay is based on the reductive cleavage of a water soluble tetrazolium salt by the dehydrogenase activity of intact mitochondria in the cells which can be quantitatively followed by a color change. The LDH assay monitors directly the activity of lactate dehydrogenase released by the cells.

1. Polymer Surfaces

Glass coverslips (15×15 and 18×18 mm²) and slides (26×76 mm²) were cleaned, hydrophobized, PSf spin-coated, and ABMPEG 10 kDa modified as described in Example 3.1 and 3.2. Surfaces were stored in 0.02% NaN₃ solution, and before plating with cells washed with distilled water and immersed into 70% ethanol for 10 sec followed by air-drying under sterile conditions. Cover slips (15×15 mm²) were inserted into 12-well-plates, larger cover slips into 6-well-plates. Slides were comparted by applying a Flexiperm silicon mask dividing the polymer surface into 8 wells.

2. Cells and Proliferation Studies

HF were cultivated and harvested as described in Example 6.1. For HUVEC and C3A other culture media were employed (HUVEC: endothelial cell growth medium, C3A: MEM) but otherwise grown and harvested in the same way as HF. After centrifugation and resuspension the cell number was counted in a Neubauer counting chamber. For cell proliferation studies the following cells and densities were applied:

| | seeding area (cm²) | | |
|---|---|---|---|
| | 8-well-array | 6-well-array | |
| | 0.88 | 9.08 | cells/cm² |
| HF | 20,000/well | | 22,700 |
| HUVEC | | 50,000/well | 5,500 |
| C3A | | 200,000/well | 22,000 |

Cells were seeded into the wells and incubated at 37° C. and 5% CO₂ up to 7 days. After 3, 5 and 7 days samples were inspected visually and their morphology/state was documented by phase-contrast photographs. After 1, 3 and 7 days XTT and LDH assays were performed.

3. Results

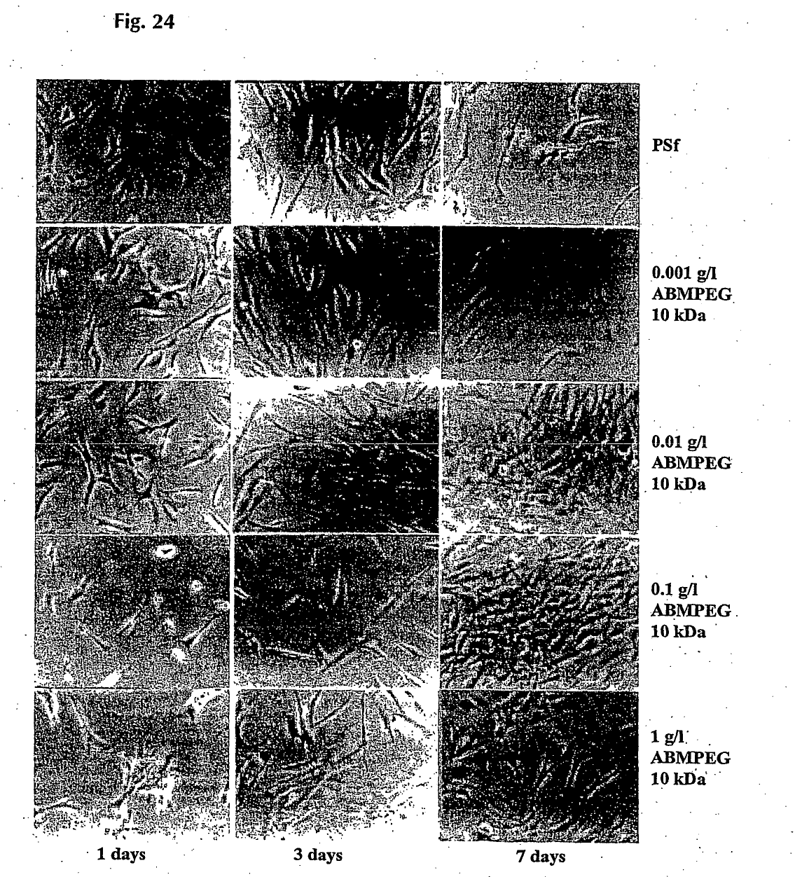

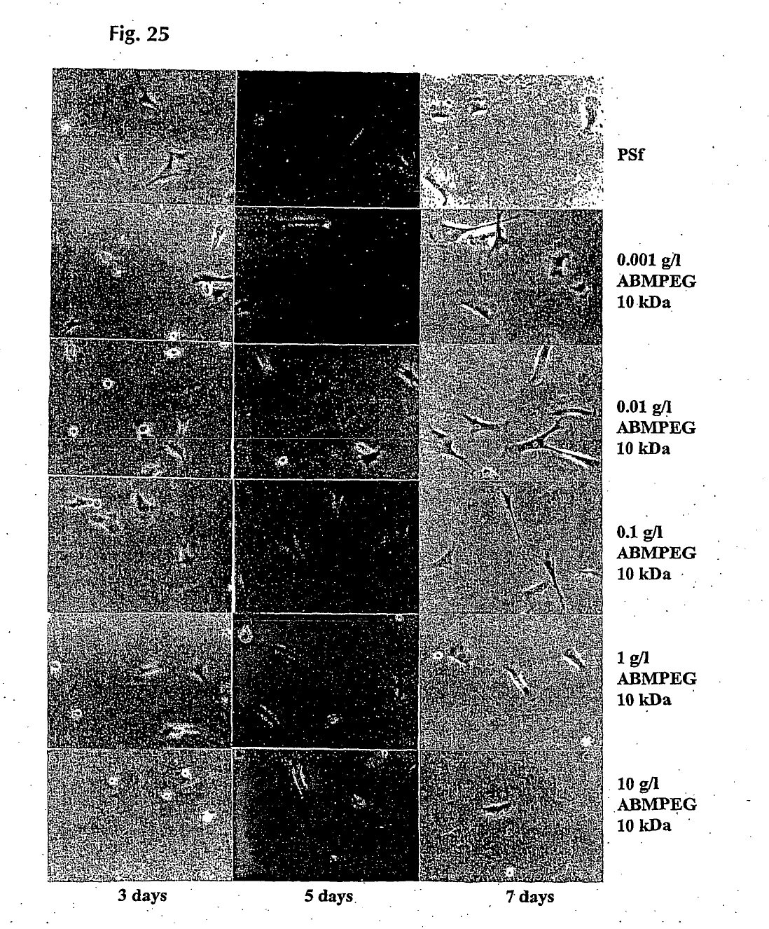

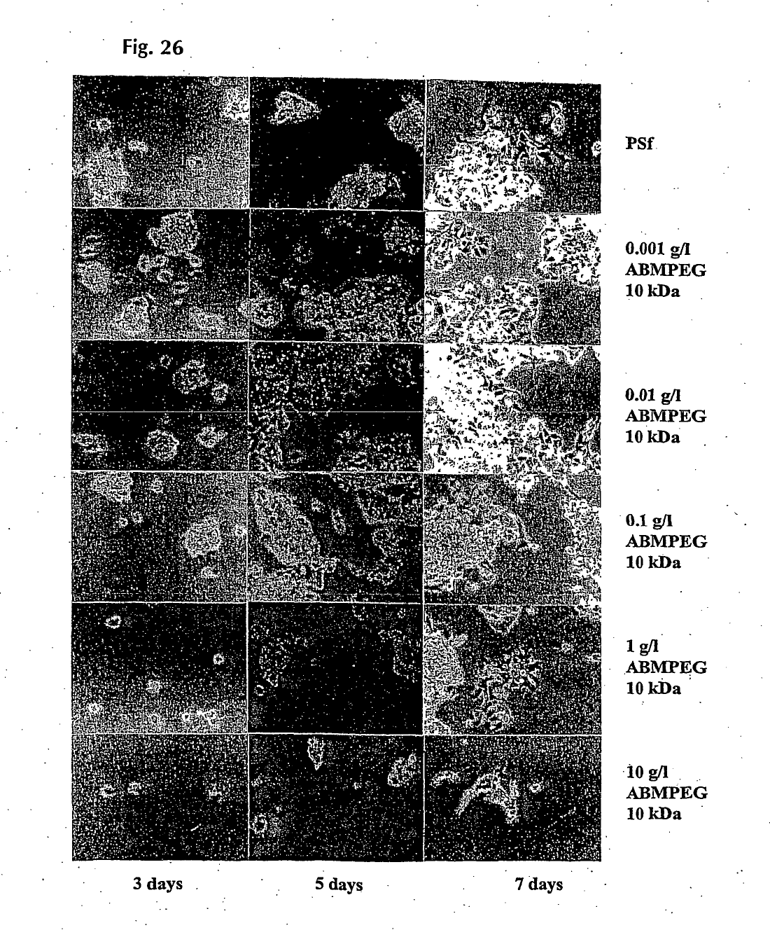

For all three cell types the best growth conditions were observed on PSf modified with ABMPEG 10 kDa at concentrations ranging from 0.01 g/l to 60.1 g/l (see FIG. 24 for HF, FIG. 25 for HUVEC, and FIG. 26 for C3A). Both, HF and C3A proliferated very well during the 7 day cultivation period and overgrew almost the whole substratum area for the named intermediate ABMPEG concentrations. HF were even found to grow in multilayers. The number of HUVEC was lower because of less initial seeding density (only 5,500 cells/cm² instead of more than 22,000 cells/cm² for HF and HUVEC).

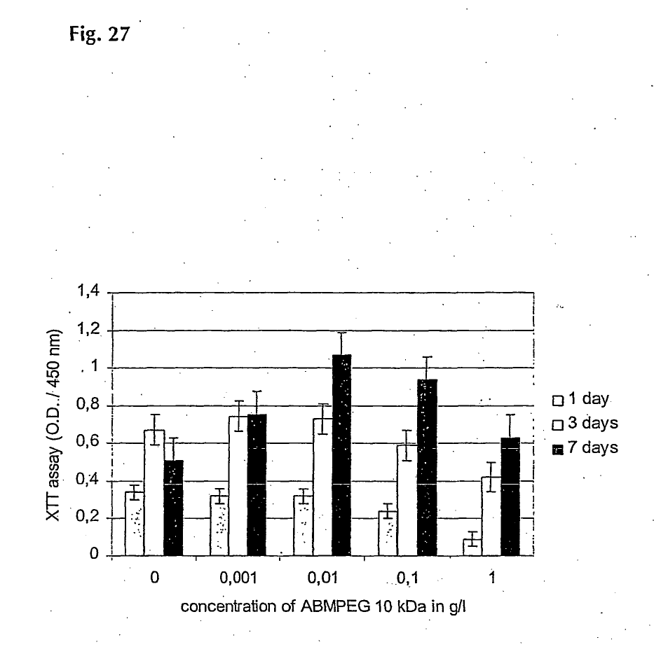

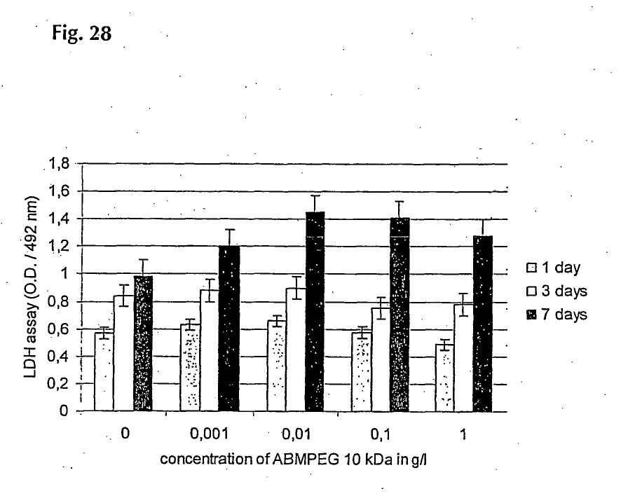

The XTT and LDH assays performed for HF confirmed the observed trend (see FIG. 27 and FIG. 28), i.e. a maximum of proliferation for PSf surfaces modified at intermediate degrees of ABMPEG 10 kDa concentration. However, these assays show a much less pronounced maximum as compared with the phase contrast photographs. This was most likely due to boundary effects originating from the used Flexiperm silicon wells. Pronounced cellular adherence and proliferation was observed for the contact line of the silicon with the underlying PSf substratum.

### Example 9

Focal Adhesion Formation of Endothelial Cells on Unmodified and ABMPEG 10 kDa Modified PSf

The development of focal adhesions is a measure for the quality of interactions between cells and interfaces (cf. Example 6).

1. Cell Cultivation

The studied surfaces were pre-coated with FN (for details see Ref. [13]). HUVEC were cultivated and plated as described in Example 8. Immunofluorescence studies were carried out as described in Example 6.3 in order to visualize points of focal adhesion between HUVEC and the underlying substratum. After incubation for 2 h, cells were inspected by phase-contrast microscopy and afterwards fixed with, 3% paraformaldehyde in PBS for 15 min. The further characterizations were performed as described in Example 6.3.

2. Results

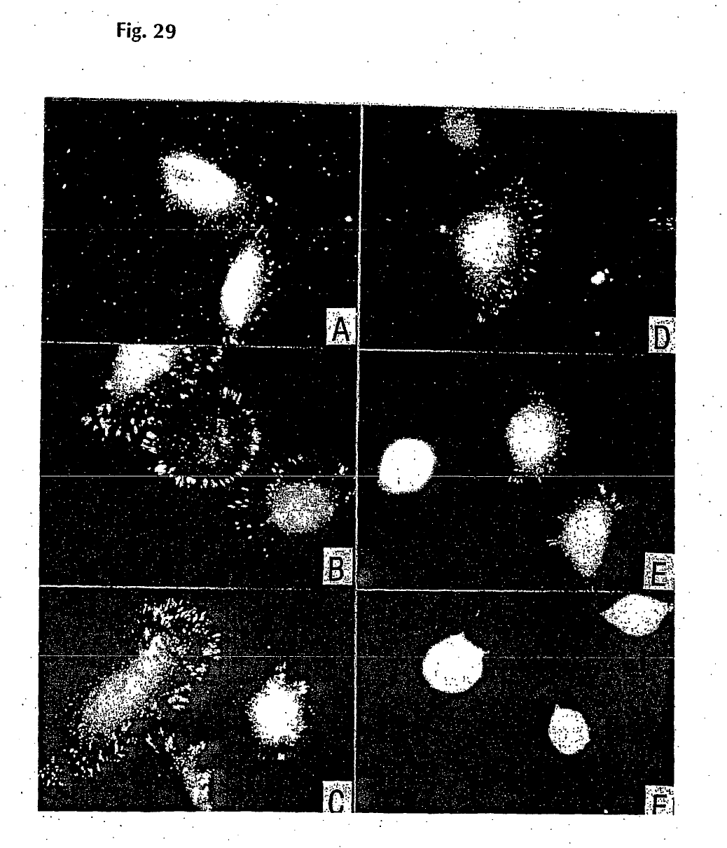

FIG. 29 clearly shows the same dependence of the Formation of focal adhesions on the degree of ABMPEG 10 kDa modification of the PSf substratum already seen before in the proliferation and adhesion studies. HUVEC plated on PSf substrata modified at intermediate concentrations of ABMPEG exhibit the highest number of focal adhesions and the best developed ones.

### Example 10

Ellipsometric Evaluation of the Interactions of Proteins, Adsorbed on Different Surfaces, with their Respective Antibodies.

The binding of antibodies to their respective antigens is only effective when both, the antigen as well as the antibody, are present in their native or biological active conformation. Antigen/antibody-binding usually represents a tight bond comprising a high dissociation constant. As discussed before, upon adsorption proteins often loose their conformational integrity and thus also their ability to bind to respective antibodies.^{[44]} Several studies have characterized these changes in conformation/antibody-binding by monitoring either the release of bound antigen,^{[45,46]} or the binding of the respective antibody to the previously adsorbed antigen e.g. by ellipsometry.^{[47,48]}

In this example we represent data which shows the increasing binding affinity of adsorbed proteins (antigens) in respect to their antibodies when these antigens were adsorbed to previously ABMPEG-modified interfaces. As a model system we choose bovine immuno globulin (BGG) and an enzyme-labeled anti-BGG(H+L), where the latter is an antibody directed towards the heavy and the light chains of BGG, i.e. towards four independent epitopes present on each BGG molecule. Furthermore, we applied human serum albumin (HSA) as blocking agent in order to cover remaining surface adsorption sites before exposing the anti-BOG to previously adsorbed BGG.

1. Preparation of Surfaces.

Hydrophilic, hydrophobic and PSf-spin-coated silicon slides were prepared as described in Example 2.1. PSf-spin-coated slides were grafted at a range of grafting densities with ABMPEG 10 kDa as described in Example 3.2.

2. Materials

BGG-FTC: Fluorescein (FMC)-conjugated ChromPure BGG, (lot 001-090-003, Jackson ImmunoResearch Laboratories, Inc.; purified over a-BGG(H+L)-column); i.e. the Antigen, abbreviated with BGG
a-BGG(H+L)-HRP: Horseredish peroxidase (HRP)-conjugated Goat Anti-Bovine BGG(H+L) from pooled antisera from goats hyperimmunized with BGG (Southern Biotechnology Associates, Inc.); i.e. the antibody, abbreviated with a-BGG
HSA: Human Serum Albumin (Centeon Pharma GmbH); i.e. the blocking agent
   3. Ellipsometric Determination of the Consecutive Adsorption of i) BGG as Antigen, ii) HSA as Blocking Agent, and iii) a-BGG as Respective Antibody to BGG to Unmodified and ABMPEG Modified PSf-Spin-Coated Silicon Slides

The instrumental set-up and the principal measurement procedure are described in Example 2.2. The differently modified PSf-spin-coated silicon slides were placed in the quartz cuvette and stabilized in 2.5 ml phosphate buffered saline (PBS; 154 mM NaCl, 10 mM Na₂HPO₄/NaH₂PO₄, pH=7.4) until constant polarizer and analyzer signals were obtained. The previously purified proteins, BGG, a-BGG, and HSA, were reestablished in PBS. At the start of each experiment (0 min), 0.5 ml of BGG-FITC solution were added to the cuvette yielding 3 ml with a defined concentration of 0.01 g/l. The contents of the cuvette was constantly stirred by a magnetic stirrer during all experiments. After 30 min of BGG adsorption the cuvette was flushed for 1 min with PBS, using preinstalled tubings and a flow rate of 20 ml/min. Thereafter, the total volume in the cuvette was readjusted to 2.5 ml. After another minute (period for signal stabilization), 0.5 ml of the concentrated HSA solution were added (at 32 min) yielding 3 ml with a defined concentration of 3 g/l. After 10 min of HSA adsorption the cuvette was flushed with PBS and the total volume readjusted as described above. At 44 min, 0.25 ml of the concentrated a-BGG solution were added yielding 2.75 ml with a defined concentration of 0.015 g/l. After 60 min of a-BGG adsorption (at 104 min) the cuvette was flushed with PBS buffer for 2 min and finally signal stabilization awaited for 1 min. At 107 min the experiment ended.

Polarizer and analyzer data were automatically collected during the whole period and the corresponding Ψ and Δ values directly calculated. Relative changes in the calculated Ψ signal (the change in Ψ signal is proportional to the total mass adsorbed) during the periods of stabilization in PBS were used to compare adsorbed amounts of the different consecutively adsorbed proteins. All presented data is the arithmetic average of two independent experimental runs.

3. Different Control Experiments

Consecutive adsorption of BGG, HSA and a-BGG, as described in the previous paragraph, was also performed on hydrophobic and hydrophilic silicon slides.

On some selected ABMPEG modified PSf-spin-coated silicon slides the first adsorption step, i.e. the adsorption of the antigen BGG, was not performed. However, adsorption of HSA and a-BGG was performed as described in the previous paragraph.

4. Results

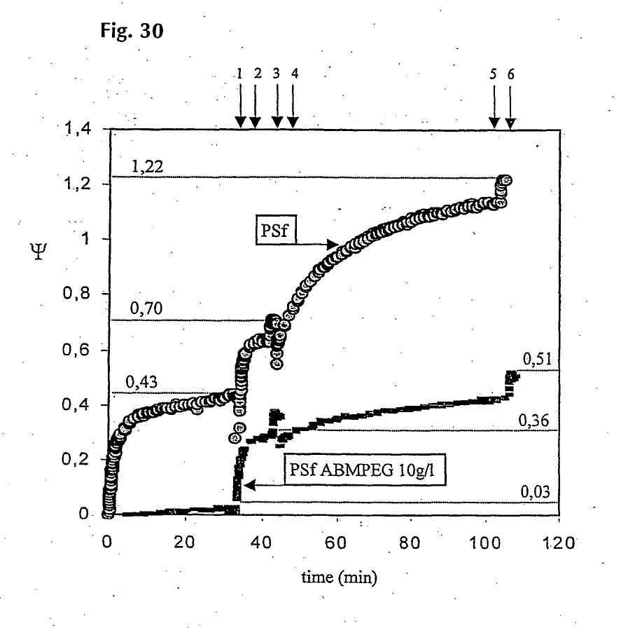

The monitored Ψ signal of two entire adsorption experiments, i.e. the consecutive adsorption of BGG, HSA and a-BGG, to unmodified PSf and PSf modified with 10 g/l ABMPEG, is shown in FIG. 30.

For the unmodified PSf, the BGG adsorption kinetic levels off to an almost constant value of Ψ=0.40 after about 20 min. After 30 min. as indicated by the first arrow, PBS flushing started, followed by the addition of HSA (second arrow) intended to work as an blocking agent to cover residual surface area not-covered by BGG. The applied concentration of HSA was 300 times higher than the applied. BGG concentration, however, between the two PBS flushings (first and third arrow), Ψ rose only by 0.27 units, indicating that the polymer interface was already substantially saturated by BGG. Furthermore, the fast leveling off of the Ψ-signal upon addition of HSA, a protein of smaller size and higher adhesiveness than BGG, indicates the efficient blocking of residual uncovered interface area. The addition of a-BGG (fourth arrow), again at a low concentration, followed by flushing (between fifth and sixth arrow) yielded however a substantial rise in the signal (0.52 units). This rise is attributed to the high affinity antigen-antibody binding, yielding a second protein layer on top of the adsorbed BGG/HSA layer. The comparatively slow kinetics of the a-BGG binding is a further indication for a different mechanisms of binding of this second layer, i.e. a antigen-antibody binding vs. adsorptive binding.

For the PEG-modified PSf-ABMPEG 10 g/l, the BGG adsorption proceeds much slower and to a far smaller extent (up to 0.035 units). The already present and covalently fixed ABMPEG reduce the available surface area and also reduce the speed of adsorption, due steric hindrance of ABMPEG moieties towards approaching BGG molecules. HSA, as mentioned a much smaller and more adhesive protein than BGG, is however less restrained of adsorbing in between the already present ABMPEG moieties, and thus adsorbing to a similar extent (0.33 units) as on PSf only covered by BGG (see above). The consecutive binding of a-BGG is comparatively smaller (0,15 units) than for the unmodified PSf (0.52 units). However, not the total amount of a-BGG bound to the surface characterizes the binding affinity of the previously adsorbed BGG, but the ratio between bound a-BGG and BGG. This ratio, however, increases in this experimental run from 1.2 for unmodified PSf to 4.3 for the PEG modified PSf-ABMPEG 10 g/l indicating a much higher binding affinity and thus higher conformational integrity or biological activity of BGG when adsorbed to PEG-modified PSf.

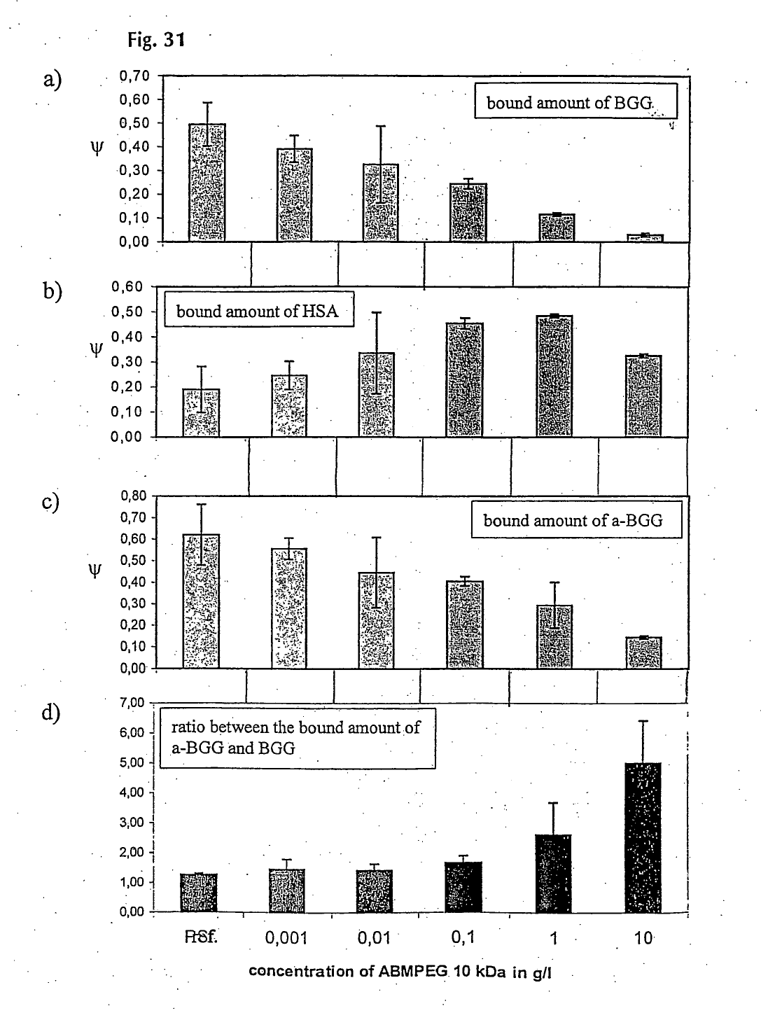

FIG. 31(a-c) shows the arithmetic mean of the relative rise in S-signal for the consecutive adsorption of BOG, HSA, and a-BGG for all performed experiments on unmodified PSf (ref.) and ABMPEG-modified PSf. The error bars represent the standard deviation of the duplicated experimental runs. As expected, BGG adsorption decreases with increasing ABMPEG grafting density by approx. 0.95% from 0.5 to 0.03 Ψ-units (FIG. 31(a)). Consecutive adsorption of comparatively highly concentrated HSA yields a maximum for PSf modified with ABMPEG at a concentration of 1 g/l (FIG. 31(b)). a-BGG adsorption decreases with increasing ABMPEG grafting density by approx. 75% from 0.62 to 0.15 Ψ-units (FIG. 31(c)). Consequently rises the ratio between the adsorbed amount of a-BGG and previously adsorbed BGG with increasing ABMPEG grafting density from 1.25 for PSf (ref.) to 5.0 (the latter values being arithmetic means of two independent experimental runs) for PSf-ABMPEG-10 g/l (FIG. 31(d)).

Control experiments with hydrophobic wafers yielded similar results as observed for unmodified PSf (BGG=0.39±0.08, HSA=0.17±0.02, a-BGG=0.40±0.05), the ratio between a-BGG and BGG being 1.03±0.08, i.e. slightly lower than for PSf. Control experiments performed on hydrophilic wafers did not yield conclusive results as the total amount of adsorbed proteins was too low.

In order to verify the efficiency of the blocking agent, HSA, ABMPEG modified PSf slides (modified at 0.01 and 1.0 g/l ABMPEG) were directly exposed to HSA under the same conditions as in the previous adsorption experiments, i.e. for 10 min to PBS buffered HSA solution of 3 g/l. Consecutive exposure to a-BGG solution (following the above procedure, i.e. 0.015 g/l a-BGG, time of adsorption: 60 min) yielded for both surfaces a slight increase of the Ψ-signal (0.1 units). However, when comparing this increase with the increase obtained in the presence of previously adsorbed BGG (see FIG. 31(c)), a much more pronounced a-BGG adsorption is observed, yielding on average an increase of 0.38 units, i.e. a four fold higher value.

Several conclusions can be drawn from this data:
i) BGG adsorption to ABMPEG modified PSf surface decreases for higher degrees of ABMPEG surface modification (see FIG. 31(a)), as also observed for FN and BSA in example 5. This confirms that independent of the adsorbing protein (FN, BSA, or BGG), increasing ABMPEG grafting density decreases protein adsorption to these surfaces.
ii) Under the assumptions that, a) HSA does effectively block the interface available for additional adsorption, and that b) a-BGG does not bind to a great extent to neither adsorbed HSA (as shown) nor grafted ABMPEG (can be assumed, see also Ref.[14] and [33]), an increase in the ratio of adsorbed a-BGG with respect to adsorbed BGG can be interpreted as an increase of conformational integrity or biological activity of the adsorbed BGG.

### REFERENCES

[1] Williams, D. F. Definitions in biomateials, Elsevier, Amsterdam (1987).
[2] Saltzman, W. M., Cell interactions with polymers, in: Principles of tissue engineering, 1^{st} ed., R. Lanza R. G. Landes Company, (1997), 225ff.
[3]: Colton, C. K., Implantable biohybrid artificial organs, Cell Transplantation 4, 415 (1995).
[4]. Andrade, J. D., Hlady, V. Protein Adsorption and Materials Biocompatibility: A Tutorial Review and Suggested Hypotheses, Adv.Polym.Sci. 79, 1 (1986).
[5] Norde, W., Lyklema, J., Why proteins prefer interfaces, J. Biomater. Sci. Polymer Edn. 2, 183 (1991).
[6] Grinnell, F., Cellular adhesiveness and extracellular substrata, Int Rev. Cytol. 53, 65 (1978).
[7] Grinnell, F., Fibronectin adsorption on material surfaces, in: Blood in contact with natural and artificial surfaces, 1^{st} ed, E. F. Leonard Ann. N.Y. A. Sci. New York (1987), 280ff.
[8] Grinnell, F., Feld, M., Fibronectin adsorption on hydrophilic and hydrophobic surfaces detected by antibody binding and analyzed during cell adhesion in serum containing medium, J. Biol. Chem. 257, 4888 (1982).
[9] Hynes, R. O., Integrins: A family of cell surface receptors, Cell 48, 549 (1987).
[10] Hynes, R. O., Integrins: Versatility, modulation and signaling, Cell 69, 11 (1992).
[12] Juliano, D. J., Saavedra, S. S., Truskey, G. A., Effect of the conformation and orientation of adsorbed fibronectin on endothelial cell spreading and the strength of adhesion, J. Biomed. Mat. Res. 27, 1103(1993).
[13] Groth, T., Zlatanov, I., Altankov, G., Adhesion of human peripheral lymphocytes on biomaterials preadsorbed with fibronectin and vitronectin, J. Biomater. Sci. Polymer Ed. 6, 729 (1994).
[14] Lee, J. H., Lee, J. W., Khan G., Lee, H. B., Interactions of cells on chargeable functional group gradient surfaces, Biomaterials 18, 351 (1997).
[15] Fabrizius-Homan, D. J., Cooper, S L., A comparison of the adsorption of three adhesive proteins to biomaterial surfaces, J. Biomater. Sci. Polymer Edn. 3, 27 (1991).
[16] Massia, S. P., Hubbell, J. A, Covalent surface immobilization of Arg-Gly-Asp- and Tyre-Ile-Gly-Ser-Arg-containing peptides to well defined cell adhesive substrates, Analy. Biochem. 187, 155 (1990).
[17] Braber den, E. T., Ruijter de, J. E., Smits, H. T. J., Ginsel, L. A., Recum, A. F., Jansen, J. A., Effect of parallel surface microgrooves and surface energy on cell growth, J. Biomed Mater Res 29, 511 (1995).
[18] Curtis, A. Wilkinson, C., Topographical control of cells, Biomaterials 18, 1573 (1997).
[19] USA, 5128170, (1992), Matsuda, T., Inoue, K, Kani, N.
[21] USA, 5330911, (1994), Hubbell, J. A Massia, S. P., Desai, N. P.
[20] USA, 5476509, (1995), Keogh, J. R.
[23] USA, 5512474, (1996), Clapper, D. L., Hu, W. S.
[22] USA, 5593814, (1997), Matsuda, T., Inoue, K., Tani, N.
[24] Yan M, Cai S X, Wybourne M N, Keana J F W. Photochemical functionalization of polymer surfaces and the production of biomolecule-carrying micrometer-scale structures by deep-UV lithography using 4-substituted perfluorophenyl azides. J Am Chem Soc 115, :814-816 (1993).
[25] Vargo T G, Thompson P M, Gerenser U J, Valentini R F, Aebischer P, Hook D J, Gardella J. Monolayer chemical lithography and characterization of fluoropolymer films. Langmuir 8, 130-134(1992).
[26] Roberts. C, Chen C S, Mrksich M, Martichonok V, Ingber D E, Whitesides G M. Using mixed self-assembled monolayers presenting RGD and (EG)3OH groups to characterize long-term attachment of bovine capillary endothelial cells to surfaces. J Am Chem Soc 120, 6548-6555 (1998).
[27] Wollman E W, Kang D, Frisbie C D, Lorkovic I M, Wrighton M S. Photosensitive self-assembled monolayers on gold: photochemistry of surface-confined aryl azide and cyclopentadienylmanganese tricarbonyl. J Am Chem Soc 116, 4395-4404 (1994).
[28] Amos R A, Guire P E, Surface modification of polymers by photochemical immobilization—a general method. 175. Scottsdale, Ariz., USA, Society of Biomaterials. 1-5-1991.
[29] Garbassi, F., Morra, M., Occhiello, E. Modification techniques, in: Polymer surfaces—from physics to technology, Anonymous. John Wiley & Sons Ltd, Chapter 6-8, Chichester (England) 221ff. (1994).
[30] Ratner B D, Surface modification of polymeric biomaterials. 1. New York, Plenum Press (1.997).
[31] Ulbricht, M., Papra, A. Polyacrylonitile enzyme ultrafiltration membranes prepared by adsorption, crosslinking, and covalent binding, Enzyme Microb. Technol. 20, 61(1997).
[32] Szleifer, I. Polymers and proteins: interactions at interfaces, Current Opinion in Solid State & Materials Science 2, 337 (1997).
[33] Sofia, S. J., Premnath, V., Merrill, E. W. Poly(ethylene oxide) grafted to silicon surfaces: grafting density and protein adsorption, Macromolecules 31, 5059 (1998).
[33] Sofia, S. J., Premnath, V., Merrill, E. W. Poly(ethylene oxide) grafted to silicon surfaces: grafting density and protein adsorption, Macromolecules 31, 5059 (1998).
[34] Schröen, C. G. P. H., Cohen Stuart, M. A., Voort Maarschalk van der, K., Padt van der, A., Riet van't, K. Influence of preadsorbed block copolymers on protein adsorption surface properties, layer thickness, and surface coverage, Langmuir 11, 3068 (1995).
[35] Chan, C. M., Contact angle measurements, in: Polymer surface modification and characterization, 1^{st} ed., Hanser Publishers, München, Chapter 2, 193ff. (1994).
[36] Cuypers, P. A., Corsel, J. W., Janssen, M. P., Kop, J. M. M., Hermens, W. T., Hemker, H. C. The adsorption of prothrombin to phosphatidylserine multilayers quantitated by ellipsometry, J Biol Chem 258, 2426 (1983).
[37]. Azzam, R. M. A, Bashara, N. M. Ellipsometry and polarized light, 3^{rd} ed., Elsevier Science, Amsterdam (1987).
[38] Arnebrant, T., Ph.D.-thesis, Lund University, Lund, Sweden, (1987).
[39] Ulbricht, M., Hicke, H. G., Photomodifizierung von Ultrafiltrationsmembranen, I. Photochemische Modifizierung von PolyacrylnitrilUltrafiltrationsmembranen mit Arylaziden, Angew. Makromol. Chem. 210, 69 (1993).
[40] Ulbricht, M., Hicke, H. G., Photomodifizierung von Ultrafiltrationsmembranen, II. Ulitrafiltrationseigenschaften von mit Arylaziden modifizierten Polyacril-Ultrafiltrations Membranen, Angew. Makromol. Chem. 210, 97 (1993).
[41] McCrackin, F. L., Passaglia, E., Stromberg, R R., Steinberg, H. L., J Res Nat Bur Stand Sect A 67, 363 (1963).
[42]. Barkholt, V., Jensen, A. L., Amino acid analysis: determination of cysteine plus half-cystine in proteins after hydrochloric acid hydrolysis with a disulfide compound as additive, Analy. Biochem. 177, 318 (1989).
[43] Pethig, R. Dielectric and electronic properties of biological materials, New York, Wiley (1979).
[44] Nygren H, Stenberg M. Immunochemistry at interfaces. Immunology 66, 321-327 (1989).
[45] Chinn J A, Posso S E, Horbett T A, Ratner B D. Postadsorptive transitions in fibrinogen adsorbed to polyurethanes: changes in antibody binding and sodium dodecyl sulfate elutability. J Biomed Mater Res 26, 757-778. (1992).
[46] Rapoza R J, Horbett T A. Postadsorptive transitions in fibrinogen: influence of polymer properties. J Biomed Mater Res 24, 1263-1287 (1990).
[47] Nygren H. Real-time recording of antigen-antibody reactions at surfaces: interpretation of data and a statistical model. Colloids Surf, B 4, 243-250 (1995).
[48] Nygren H, Kaartinen M, Stenberg M. Determination by ellipsometry of the affinity of monoclonal antibodies. J Immunol Methods 92, 219-225 (1986).

1. Biocompatible material comprising a substratum contacted by at least one macromolecule,
said material having a first advancing contact angle a,
said substratum having a second advancing contact angle b₀ when not contacted by a macromolecule, and another second advancing contact angle bₛₐₜ, when said substratum is saturated by said macromolecules,
wherein said advancing contact angles are measured using water and air saturated by water vapour,
wherein bₛₐₜ essentially does not change when the substratum is contacted by further macromolecules by means of a chemical bond,
wherein the relation between said advancing contact angles is as defined by the ratio R,
R=(b₀−a)/(b₀−bₛₐₜ)
and wherein the numerical value of R is in the interval from 0 to less than 0.4.

2. Material according to claim 1, wherein said substratum is selected from the group consisting of poly(lactide) (PLA), poly(glycolic acid) (PGA), poly(lactide-co-glycolide) (PLGA), poly(caprolactone), polycarbonates, polyamides, polyanhydrides, polyamino acids, polyortho esters, polyacetals, polycyanoacrylates and degradable polyurethanes.

3. Material according to claim 1, wherein said substratum is selected from the group consisting of polyacrylates, ethylene-vinyl acetate polymers and other acyl substituted cellulose acetates and derivatives thereof, non-erodible polyurethanes, polystyrenes, polyvinyl chloride, polyvinyl fluoride, poly(vinyl imidazole), chlorosulphonated polyolifins, polyethylene oxide, polyvinyl alcohol, teflon, and nylon.

4. Material according to claim 1, wherein said substratum is selected from the group consisting of homo- and copolymers of linear low density polyethylene (LLDPE), Low density polyethylene (LDPE), High density polyethylene (HDPE), Ethylene/vinylacetate (EVA), Ethylene-methyl-acrylate (EMA), Ethylene-acrylic-acid (EAA), Ethylene-butyl-acrylate (EBA), Ethylene-ethyl-acrylate (EEA), Polypropylene (PP), Ethylene-propylene copolymer (EPM), and Ethylene-propylene-diene terpolymer (EPDM).

5. Material according to claim 1, wherein said substratum is selected from the group consisting of polyethylene (PE), high density polyethylene (HDPE), low density polyethylene (LDPE), polypropylene (PP) and poly(4-methyl-1-pentene) (PMP).

6. Material according to claim 1, wherein said substratum comprises or essentially consists of or consists of a polycarbonate, or a derivative thereof.

7. Material according to claim 1, wherein said substratum comprises or essentially consists of or consists of a polystyrene, or a derivative thereof.

8. Material according to claim 1, wherein said substratum comprises a hydrophobic polymer.

9. Material according to claim 1, wherein said substratum has an advancing contact angle of more than 90 degrees.

10. Material according to claim 1, wherein said substratum is pretreated or modified, wherein said pretreatment or modification results in an increased wettability of the substratum.

11. Material according to claim 10 wherein said pretreated or modified substratum is the result of said substratum being contacted by and/or operably linked to a charged group or a hydrophilic compound.

12. Material according to claim 1, wherein said substratum is subjected to a pretreatment comprising corona treatment and resulting in an increased wettability of said substratum.

13. Material according to claim 1, wherein said substratum is subjected to a pretreatment comprising plasma treatment and resulting in an increased wettability of said substratum.

14. Material according to claim 1, wherein the substratum is further contacted by a plurality of soluble substances capable of forming a self-assembled monolayer comprising at least one macromolecule.

15. Material according to claim 14, wherein said soluble substances are n-alkane chains preferably containing from 8 to 24 carbons.

16. Material according to claim 1, wherein said macromolecule comprises an amphiphilic polymer.

17. Material according to claim 1, wherein said first contact angle is in the range of from 50 degrees to 140 degrees.

18. Material according to claim 1, wherein said first contact angle is in the range of from 60 degrees to 125 degrees.

19. Material according to claim 1, wherein said first contact angle is in the range of from 70 degrees to 120 degrees.

20. Material according to claim 1, wherein said first contact angle is in the range of from 75 degrees to 110 degrees.

21. Material according to claim 1, wherein said first contact angle is in the range of from 80 degrees to 100 degrees.

22. Material according to claim 1, wherein said ratio is less than 0.30.

23. Material according to claim 1, wherein said ratio is less than 0.28.

24. Material according to claim 1, wherein said ratio is less than 0.26.

25. Material according to claim 1, wherein said ratio is less than 0.24.

26. Material according to claim 1, wherein said ratio is less than 0.22.

27. Material according to claim 1, wherein said ratio is less than 0.20.

28. Material according to claim 1, wherein said ratio is less than 0.18.

29. Material according to claim 1, wherein said ratio is less than 0.16.

30. Material according to claim 1, wherein said ratio is less than 0.14.

31. Material according to claim 1, wherein said ratio is less than 0.12.

32. Material according to claim 1, wherein said ratio is less than 0.10.

33. Material according to claim 1, wherein said material, when contacted by a first determinant comprising a compound selected from the group consisting of a polypeptide, or part thereof, a nucleic acid moiety, a carbohydrate moiety, and a lipid moiety, including any combination thereof, is capable of maintaining said compound in a biologically active form.

34. Material according to claim 33 wherein said compound is a polypeptide or part thereof.

35. Material according to claim 33 further comprising said first determinant comprising said compound, wherein said first determinant is maintained in a biologically active form when contacted by said substratum and/or said macromolecule.

36. Material according to claim 35 wherein said biologically active form is essentially a biologically active conformation.

37. Material according to claim 33 wherein said biologically active form or conformation is maintained and/or improved and/or stabilized by means of the cooperativity of said substratum and said macromolecule.

38. Material according to claim 33 wherein said biologically active form or confirmation is maintained and/or improved and/or stabilized when contacted by said substratum and said macromolecule.

39. Material according to claim 1, wherein said material is biocompatible.

40. Material according to claim 1, wherein the weight increase per area unit arising from the part of the macromolecule essentially consisting of PEG or poly(ethylene oxide) (PEO) is less than 2.0×10⁻²² grams (g) per square nanometer (nm²).

41. Material according to claim 40, wherein said difference is less than 1.6×10⁻²² grams (g) per square nanometer (nm²).

42. Material according to claim 40, wherein said difference is less than 1.4×10⁻²² grams (g) per square nanometer (nm²).

43. Material according to claim 40, wherein said difference is less than 1.2×10⁻²² grams (g) per square nanometer (nm²).

44. Material according to claim 40, wherein said difference is less than 1.0×10⁻²² grams (g) per square nanometer (nm²)

45. Material according to claim 40, wherein said difference is less than 0.8×10⁻²² grams (g) per square nanometer (nm²).

46. Material according to claim 40, wherein said difference is less than 0.5×10⁻²² grams (g) per square nanometer (nm²).

47. Material according to claim 40, wherein said difference is less than 0.3×10⁻²² grams (g) per square nanometer (nm²).

48. Material according to claim 1 wherein each macromolecule is associated with an excluded volume.

49. Material according to claim 48, wherein said substratum is at least substantially flexible.

50. Material according to claim 48, wherein said substratum is a film.

51. Material according to claim 48, wherein said substratum is essentially rigid or at least substantially non-flexible.

52. Material according to claim 51, wherein said substratum comprises a crystalline structure capable of supporting a self-assembled monolayer such as gold, silicon oxide, and similar crystalline structures and/or structures that are smooth on a nanometer scale.

53. Material according to claim 1, wherein said macromolecule has a MW of more than 400 Da.

54. Material according to claim 53, wherein said macromolecule has a MW of more than 1,000 Da.

55. Material according to claim 53, wherein said macromolecule has a MW of more than 2,000 Da.

56. Material according to claim 53, wherein said macromolecule has a MW of more than 5,000 Da.

57. Material according to claim 53, wherein said macromolecule has a MW of more than 10,000 Da.

58. Material according to claim 53, wherein said macromolecule has a MW of more than 50,000 Da.

59. Material according to claim 53, wherein said macromolecule has a MW of more than 100,000 Da.

60. Material according to claim 1, wherein said macromolecule is a conjugate comprising a head group, a guiding group, a linker group, a polymer chain or a main body, and a functional end group.

61. Material according to claim 1, wherein said macromolecule is a conjugate comprising a head group, a linker group, a polymer chain or a main body, and a functional end group.

62. Material according to claim 1, wherein said macromolecule is a conjugate comprising a head group, a polymer chain or a main body, and a functional end group.

63. Material according to claim 60, wherein said head group is capable of forming a chemical bond.

64. Material according to claim 60, wherein said head group is capable of adsorbing to the substratum.

65. Material according to claim 60, wherein said head group is capable of forming an ionic bond.

66. Material according to claim 60, wherein said head group may be entangled into or with the substratum.

67. Material according to claim 60, wherein said head group is capable of forming a self-assembled monolayer.

68. Material according to claim 60, wherein said guiding group is a bifunctional group comprising an aliphatic, linear or weakly branched group.

69. Material according to claim 61, wherein said linker group is capable of being enzymatically or chemically hydrolyzed.

70. Material according to claim 60, wherein said linker group is hydrolytically unstable and capable of being cleaved.

71. Material according to claim 60, wherein said linker group is essentially stable against cleavage under practical circumstances.

72. Material according to claim 60, wherein said polymer chain or main body is hydrophilic, uncoiling in an aqueous environment and exhibiting an excluded volume.

73. Material according to claim 60, wherein said functional end group is capable of linking permanently or reversibly other biological or synthetic molecules or materials.

74. Material according to claim 33, wherein said first determinant comprises a biologically active compound comprising a polypeptide, or a part thereof, a nucleic acid moiety, a carbohydrate moiety, and a lipid moiety, or any combination thereof.

75. Material according to claim 74, wherein said biologically active compound comprises a polypeptide.

76. Material according to claim 74, wherein said biologically active compound is a membrane associated and/or extracellular matrix polypeptide natively produced by a microbial cell, a plant cell or a mammalian cell.

77. Material according to claim 74 wherein said biologically active compound is selected from the group consisting of a polypeptide, an antibody, a polyclonal antibody, a monoclonal antibody, an immunogenic determinant, an antigenic determinant, a receptor, a receptor binding protein, an interleukine, a cytokine, a cellular differentiation factor, a cellular growth factor, and an antagonist to a receptor.

78. Material according to claim 74, wherein said biologically active compound is a synthetic polypeptide, or part thereof, capable of contacting said substratum and/or said macromolecule.

79. Material according to claim 74, wherein said biologically active compound is a synthetic polypeptide, or part thereof, capable of contacting said substratum and said macromolecule.

80. Material according to claim 74, wherein said biologically active compound is an adhesion polypeptide, preferably fibronectin or vitronectin.

81. Material according to claim 33, wherein said biologically active compound results in an improved contact between said material and a biological entity, such as a biological cell or a virus, or part thereof, including a polypeptide, or a part thereof, a nucleic acid moiety, a carbohydrate moiety, and a lipid moiety, or any combination thereof.

82. Material according to claim 1, said material further comprising a second determinant.

83. Material according to claim 82, wherein said second determinant comprises a biological entity, such as a biological cell or a virus, or part thereof, including a polypeptide, or a part thereof, a nucleic acid moiety, a carbohydrate moiety, and a lipid moiety, or any combination thereof.

84. Material according to claim 82, wherein said biological entity is selected from the group consisting of a polypeptide, an antibody, a polyclonal antibody, a monoclonal antibody, an immunogenic determinant, an antigenic determinant, a receptor, a receptor binding protein, an interleukine, a cytokine, a differentiation factor, a growth factor, and an antagonist to the receptor.

85. Material according to claim 83, wherein said biological cell, or part thereof, is selected from the group consisting of a mammalian cell, a plant cell, and a microbial cell.

86. Material according to claim 85 wherein said biological cell is a mammalian cell.

87. Material according to claim 83, wherein said virus, or part thereof, is selected from a mammalian virus, a plant virus, and a microbial virus.

88. Material according to claim 87 wherein said virus is a mammalian virus.

89. Material according to claim 1, wherein said substratum is porous.

90. Material according to claim 89, wherein the flux of water through said material is substantially unchanged as compared to the flux of water through said porous substratum.

91. Material according to claim 1, wherein said substratum is non-porous and/or substantially non-penetrable to water.

92-100. (Cancelled)

101. Composition comprising the material according to claim 1 and a physiologically acceptable carrier.

102. Pharmaceutical composition comprising the material according to claim 1 and a pharmaceutically active ingredient and optionally a pharmaceutically active carrier.

103-114. (Cancelled)

115. Method of controlling cellular growth and/or cellular proliferation and/or cellular differentiation ex vivo, said method comprising the steps of contacting a cell with the material according to claim 1 and incubating said cell and said material under conditions allowing said cell to grow and/or proliferate and/or differentiate.

116. Method of separating and/or isolating biological material ex vivo, said method comprising the steps of contacting said biological material to be separated and/or isolated with the material according to claim 1 and incubating said biological material and said material under conditions that allow separation and/or isolation.

117. Method of producing a biohybrid organ ex vivo, said method comprising the steps of contacting biohybrid organ cells with the material according to claim 1 and incubating said biohybrid organ cells under conditions allowing the production of said biohybrid organ.

118. Method of therapy carried out on the human or animal body, said method comprising the step of contacting said body with the pharmaceutical composition according to claim 102.

119. Method of surgery carried out on the human or animal body, said method comprising the step of contacting said body with the pharmaceutical composition according to claim 102.

120. Method of diagnosis carried out on the human or animal body, said method comprising the steps of contacting said body with the material according to claim 1 and detecting a signal generated directly or indirectly by said material.

121. Method of controlling cellular growth and/or cellular proliferation and/or cellular differentiation in vivo, said method comprising the steps of contacting a cell with the material according to claim 1 and incubating said cell and said material under conditions allowing said cell to grow and/or proliferate and/or differentiate.

122. Method of separating and/or isolating biological material in vivo, said method comprising the steps of contacting said biological material to be separated and/or isolated with the material according to claim 1 and incubating said biological material and said material under conditions that allow separation and/or isolation.

123. Method of producing a biohybrid organ in vivo, said method comprising the steps of contacting biohybrid organ cells with the material according to claim 1 and incubating said biohybrid organ cells under conditions allowing the production of said biohybrid organ.

124. Method of in vivo delivery of a medicament to a human or animal body in need of said medicament, said method comprising the steps of contacting said body with the pharmaceutical composition according to claim 102 and incubating said body contacted by said pharmaceutical composition under conditions allowing delivery of said medicament.

125. Method for producing the material according to claim 1, said method comprising the steps of i) providing a substratum having a second contact angle, and ii) contacting said substratum with a composition comprising a plurality of macromolecules and iii) providing a biocompatible material comprising a substratum contacted by a plurality of macromolecules,
wherein said material has a first advancing contact angle a,
wherein said substratum has a second advancing contact angle b₀ when not contacted by a macromolecule, and another second advancing contact angle bₛₐₜ, when said substratum is saturated by said macromolecules,
wherein said advancing contact angles are measured using water and air saturated by water vapour,
wherein bₛₐₜ essentially does not change when the substratum is contacted by further macromolecules by means of a chemical bond,
wherein the relation between said advancing contact angles is as defined by the ratio R,
R=(b₀−a)/(b₀−bₛₐₜ)
and wherein the numerical value of R is in the interval from 0 to less than 0.4

126. Method according to claim 125, wherein said substratum comprises a hydrophobic polymer.

127. Method according to claim 125, wherein said substratum is pretreated prior to being contacted by said macromolecule.

128. Method according to claim 127, wherein said pretreatment is effective in increasing the wettability of said substratum.

129. Method according to claim 125, wherein said macromolecule comprises a hydrophilic polymer.

130. Method according to claim 125, wherein said macromolecule comprises a latently reactive polymer.

131. Method according to claim 125, wherein macromolecule has a MW of more than 400 Da.

132. Method according to claim 125, wherein said macromolecule comprises a conjugate comprising a cross likable head group, a linker group, a polymer chain, and a functional end group.

133. Method according to claim 132, wherein said cross likable head group is a photo-reactive aryl azide head group.

134. Method according to claim 132, wherein said macromolecule further comprises a modifying agent.

135. Method according to claim 134 wherein said modifying agent is capable of contacting said substratum and forming a self assembled monolayer.

136. Method according to claim 125, said method comprising the further step of contacting said material with a first determinant comprising a biologically active compound.

137. Method according to claim 136, wherein said biologically active compound is selected from the group consisting of a polypeptide, an antibody, a polyclonal antibody, a monoclonal antibody, an immunogenic determinant, an antigenic determinant, a receptor, a receptor binding protein, an interleukine, a cytokine, a cellular differentiation factor, a cellular growth factor, and an antagonist to a receptor.

138. Method according to claim 136, wherein said biologically active compound is a membrane associated and/or extracellular matrix polypeptide natively produced by a microbial cell, a plant cell or a mammalian cell.

139. Method according to claim 136, said method comprising the further step of contacting said material with a second determinant comprising a biological entity.

140. Method according to claim 139, wherein said biological entity comprises a cell or a virus, or a part thereof.

141. Method according to claim 140, wherein said cell, or part thereof, is selected from the group consisting of a mammalian cell, a plant cell, and a microbial cell.

142. Method according to claim 140, wherein said virus, or part thereof, is selected from a mammalian virus, a plant virus, and a microbial virus.

143. Method according to claim 139, wherein said biological entity comprises a polypeptide, or a part thereof, a nucleic acid moiety, a carbohydrate moiety, or a lipid moiety, or any combination thereof.

144. Method according to claim 139, wherein said biological entity is selected from the group consisting of a polypeptide, an antibody, a polyclonal antibody, a monoclonal antibody, an immunogenic determinant, an antigenic determinant, a receptor, a receptor binding protein, an interleukine, a cytokine, a differentiation factor, a growth factor, and an antagonist to the receptor.
